(19) United States
(12) Patent Application Publication
(10) Pub. No.: US 2024/0099279 A1
(43) Pub. Date: Mar. 28, 2024

(54) GENETICALLY MODIFIED MICE EXPRESSING COMPONENTS OF HUMAN CELLULAR IMMUNE SYSTEM

(71) Applicant: Regeneron Pharmaceuticals, Inc., Tarrytown, NY (US)

(72) Inventors: Naxin Tu, Pleasantville, NY (US); John McWhirter, Sanremo (IT); Chunguang Guo, Briarcliff Manor, NY (US); Cagan Gurer, Chappaqua, NY (US); Vera Voronina, North Bethesda, MD (US); Lynn Macdonald, Harrison, NY (US); Andrew J. Murphy, Croton-on-Houdson, NY (US)

(21) Appl. No.: 18/472,847

(22) Filed: Sep. 22, 2023

Related U.S. Application Data
(60) Provisional application No. 63/383,213, filed on Nov. 10, 2022.
(60) Provisional application No. 63/376,706, filed on Sep. 22, 2022.

(51) Int. Cl. A01K 67/027; C07K 14/725; C12N 15/63

(52) U.S. Cl. CPC A01K 67/0278; C07K 14/7051; C12N 15/63; A01K 2217/072; A01K 2227/105

(57) ABSTRACT

Disclosed herein are non-human animals (e.g., rodents, e.g., mice or rats) genetically engineered to express a humanized or human T cell receptor (TCR) comprising a variable domain encoded by (a) at least one human TCR variable region γ gene segment and a (human) TCR γ constant region gene sequence and/or (b) or at least one human TCR variable region δ gene segment and a (human) TCR δ constant region gene sequence. Also provided are embryos, tissues, and cells expressing the same. Methods for making a genetically engineered animal that expresses the humanized or human γ and/or δ TCR are also provided. Methods for using the genetically engineered animals that mount a substantially humanized T cell immune response for developing human therapeutics are also provided.

### CROSS-REFERENCE TO RELATED APPLICATIONS

This application claims the benefit under 35 USC § 119(e) of U.S. Provisional Application Ser. No. 63/376,706, filed Sep. 22, 2022, and U.S. Provisional Application Ser. No. 63/383,213, filed Nov. 10, 2022, each of which applications is hereby incorporated by reference.

### SEQUENCE LISTING

The official copy of the sequence listing is submitted electronically via EFS-Web as an xml formatted sequence listing with a file named 11224.xml, created on Sep. 21, 2023, and having a size of 41 kilobytes, and is filed concurrently with the specification. The sequence listing contained in this xml formatted document is part of the specification and is herein incorporated by reference in its entirety.

### TECHNICAL FIELD

The present disclosure relates to non-human animals (e.g., rodents, e.g., mice or rats) that express:
(i) a human(ized) γδ T cell receptor (TCR) comprising:
   (a) a human(ized) TCR polypeptide or a human(ized) hybrid TCR polypeptide comprising a human hybrid TCR α/δ variable domain and a human TCR δ constant domain and/or
   (b) a human(ized) TCRγ polypeptide, and
(ii) an αβ TCR comprising:
   (a) an endogenous or human(ized) TCRα polypeptide, and
   (b) an endogenous or human(ized) TCRβ polypeptide, and optionally
(iii) one or more human(ized) T cell co-receptor(s) (e.g., CD4 and/or CD8 (e.g., CD8α, and/or CD8β)), and/or (iv) one or more human(ized) major histocompatibility complex(es) that associates with the one or more human(ized) T cell co-receptor(s) (e.g., MHC II complex (e.g., MHC II α and/or MHC II β) and/or MHC I (which may be e.g., a human(ized) MHC I α polypeptide alone or complexed with human(ized) β2 microglobulin)). The disclosure also relates to embryos, tissues, cells and/or nucleic acids isolated from the non-human animals; methods of making the non-human animals; and methods of using the non-human animals, e.g., as a non-human animal model to study γ and/or δ T cell responses for the development of human therapeutics.

### BACKGROUND OF THE INVENTION

In the adaptive immune response, foreign antigens are recognized by receptor molecules on B lymphocytes (e.g., immunoglobulins) and T lymphocytes (e.g., T cell receptors also referred to as TCRs).

Not all antigens will provoke T cell activation due to tolerance mechanisms. However, in some diseases (e.g., cancer, autoimmune diseases) peptides derived from self-proteins become the target of the cellular component of the immune system, which results in destruction of cells presenting such peptides. There has been significant advancement in recognizing antigens that are clinically significant (e.g., antigens associated with various types of cancer) and/or TCR sequences that bind the clinically significant antigens. However, in order to improve identification and selection of clinically significant peptides that will provoke a suitable response in a human T cell and/or of TCR capable of binding the clinically significant antigens (e.g., for adoptive immunotherapy of cancer, T cell vaccination for autoimmunity, etc.), there remains a need for in vivo and in vitro systems that mimic aspects of human immune system. Thus, there is a need for biological systems (e.g., genetically modified non-human animals and cells) that can display components of a human immune system, particularly components of the T cell immune response.

### SUMMARY OF THE INVENTION

Described herein are non-human animals (e.g., rodents (e.g., rats or mice)) with a humanized TRD gene locus (encoding a TCR-δ polypeptide) and/or TRG gene locus (encoding a TCR-γ polypeptide), which may lead to potential therapeutics utilizing human γ/δ TCR and/or T cells.

In some embodiments, a humanized TCR γ mouse as described herein comprises:
(I) germ cells and CD3⁻ somatic cells that comprise an unrearranged TCR γ variable region sequence comprising an unrearranged human TCR Vγ segment and an unrearranged human TCR Jγ segment,
   wherein the unrearranged TCR γ variable region sequence is operably linked to a human TCRγ constant region gene sequence, optionally at an endogenous TCRγ locus (e.g., a human TCRγ constant region gene sequence at an endogenous TCRγ locus, wherein a nucleotide sequence comprising an endogenous TCRγ constant region gene sequence (e.g., an endogenous Trgc1 constant region gene sequence, an endogenous Trgc2 constant region gene sequence, an endogenous Trgc3 constant region gene sequence, and/or an endogenous Trgc4 constant region gene sequence) is replaced with a nucleotide sequence comprising a human TCRγ constant region gene sequence, (e.g., a human TRGC1 constant region sequence and/or a human TRGC2 constant region sequence),
   wherein the unrearranged human TCR Vγ segment and the unrearranged human TCR Jγ segment are capable of rearranging (or rearrange) in a T cell to form a rearranged human TCR Vγ/Jγ variable region gene that is operably linked to the human TCR γ constant region gene sequence, and
   wherein the rearranged human TCR Vγ/Jγ variable region gene operably linked to the human TCR γ constant region gene sequence together encode a human TCR γ polypeptide, and
(II) a CD3⁺ T cell that expresses, on its surface a TCR that comprises the human TCR γ polypeptide.

In some humanized TCR γ mouse embodiments, the germ cells and CD3⁻ somatic cells further comprise an unrearranged T cell receptor (TCR) δ variable region sequence comprising an unrearranged human TCR Vδ segment, an unrearranged human TCR Dδ segment, and an unrearranged human TCR Jδ segment, wherein the unrearranged TCR δ variable region sequence is operably linked to a human TCR δ constant region gene sequence, optionally at an endogenous TCR δ locus, and wherein the unrearranged human TCR Vδ segment, the unrearranged human TCR Dδ segment, and the unrearranged human TCR Jδ segment are capable of rearranging (or rearrange) in a T cell to form a rearranged human TCR Vδ/Dδ/Jδ variable region gene that is operably linked to the human TCR δ constant region gene sequence, wherein the rearranged human TCR Vδ/Dδ/Jδ variable region gene operably linked to the human TCR δ constant region gene sequence together encode a human TCR δ polypeptide, wherein the mouse comprises a CD3⁺ T cell that expresses, on its surface, a functional TCR that comprises both the human TCR γ polypeptide and the human TCR δ polypeptide. In some embodiments, the germ cells and CD3⁻ somatic cells further comprise an unrearranged human TCR Vα segment upstream of the unrearranged TCR δ variable region sequence and the human TCR δ constant region gene sequence, wherein the unrearranged human TCR Vα segment, the unrearranged human TCR Dδ, and the unrearranged human TCR Jδ segment are capable of rearranging (or rearrange) in a T cell to form a rearranged human TCR Vα/Dδ/Jδ variable region gene that is operably linked to the human TCR δ constant region gene sequence, wherein the rearranged human TCR Vα/Dδ/Jδ variable region gene sequence operably linked to the human TCR δ constant region gene sequence together encode a human hybrid TCR polypeptide comprising a human hybrid TCR α/δ variable domain and a human TCR δ constant domain, and wherein the mouse comprises a CD3⁺ T cell that expresses, on its surface, a functional TCR that comprises the human hybrid TCR polypeptide comprising the human hybrid α/δ variable domain and the human TCR δ constant domain.

A humanized TCR δ mouse as described herein may comprise:
(I) germ cells and CD3⁻ somatic cells that comprise from 5′ to 3:
   an unrearranged human TCR Vα segment and
   an unrearranged TCR δ variable region sequence comprising an unrearranged human TCR Vδ segment, an unrearranged human TCR Dδ segment, and an unrearranged human TCR Jδ segment,
      wherein the unrearranged TCR δ variable region sequence is operably linked to a human TCR δ constant region gene sequence, optionally at an endogenous TCR δ locus,
      wherein the unrearranged human TCR Vα segment, the unrearranged human TCR Dδ, and the unrearranged human TCR Jδ segment are capable of rearranging (or rearrange) in a T cell to form a rearranged human TCR Vα/Dδ/Jδ variable region gene that is operably linked to the human TCR δ constant region gene sequence,
      wherein the rearranged human TCR Vα/Dδ/Jδ variable region gene sequence operably linked to the human TCR δ constant region gene sequence together encode a human hybrid TCR polypeptide comprising a human hybrid TCR α/δ variable domain and a human TCR δ constant domain, and
(II) a CD3⁺ T cell that expresses, on its surface, a functional TCR that comprises the human hybrid TCR comprising the human hybrid α/δ variable domain and the human TCR δ constant domain.

In some humanized TCR δ mouse embodiments, the germ cells and CD3⁻ somatic cells comprise a replacement of an endogenous TCR Vα segment with the unrearranged human TCR Vα segment and a replacement of an endogenous TCR Jα segment with an unrearranged human TCR Jα segment, wherein the unrearranged human TCR Vα segment and unrearranged human TCR Jα segment are operably linked to each other and a TCR α constant region gene sequence, such as a mouse TCR α constant region gene sequence, and wherein the unrearranged human TCR Vα segment and unrearranged human TCR Jα segment are capable of rearranging (or rearrange) in a T cell to form a rearranged TCR Vα/Ja variable region gene operably linked to the TCR α constant region gene sequence, wherein the rearranged human TCR Vα/Ja variable region gene operably linked to the TCR α constant region gene sequence together encode a TCR α polypeptide comprising a human TCR α variable domain operably linked to a TCR α constant domain (e.g., a human or mouse TCR α constant domain), and wherein the mouse comprises a CD3⁺ T cell that expresses, on its surface, a functional TCR comprising the TCR α polypeptide. In some humanized TCR δ mouse embodiments, the germ cells and CD3⁻ somatic cells comprise a replacement of all endogenous TCR Vα segments with a full repertoire of unrearranged human TCR Vα segments and a replacement of all endogenous TCR Jα segments with a full repertoire of unrearranged human TCR Jα segments, wherein the full repertoire of unrearranged human TCR Vα segments and full repertoire of unrearranged human TCR Jα segments are operably linked to each other and a mouse TCR α constant region gene sequence at an endogenous TCR α locus and wherein full repertoire of unrearranged human TCR Vα segments and full repertoire of unrearranged human TCR Jα segments are capable of rearranging (or rearrange) in a T cell to form a rearranged human TCR Vα/Ja variable region gene operably linked to the mouse TCR α constant region gene sequence, wherein the rearranged TCR Vα/Ja variable region gene operably linked to the mouse TCR α constant region gene sequence together encode a chimeric TCR α polypeptide comprising a human TCR α variable domain operably linked to a mouse TCR α constant domain, and wherein the mouse comprises a CD3⁺ T cell that expresses, on its surface, a functional TCR that comprises the chimeric TCR α polypeptide.

In some humanized γ and/or δ TCR mouse embodiments, the germ cells and CD3⁻ somatic cells comprise:
(A) a replacement of an endogenous TCR Vγ segment with an unrearranged human TCR Vγ segment, a replacement of an endogenous TCR Jγ segment with an unrearranged human TCR Jγ segment, and a replacement of an endogenous TCR γ constant region gene sequence with a human TCR γ constant region gene sequence; or
(B) a replacement of an endogenous TCR Vδ segment with an unrearranged human TCR Vδ segment, a replacement of an endogenous TCR Dδ segment with an unrearranged human TCR Dδ segment, a replacement of an endogenous TCR Jδ segment with an unrearranged human TCR Jδ segment, and a replacement of an endogenous TCR δ constant region gene sequence with a human TCR δ constant region gene sequence; or
(C) (i) a replacement of an endogenous TCR Vγ segment with an unrearranged human TCR Vγ segment, a replacement of an endogenous TCR Jγ segment with an unrearranged human TCR Jγ segment, and a replacement of an endogenous TCR γ constant region gene sequence with a human TCR γ constant region gene sequence, and
   (ii) a replacement of an endogenous TCR Vδ segment with an unrearranged human TCR Vδ segment, a replacement of an endogenous TCR Dδ segment with an unrearranged human TCR Dδ segment, a replacement of an endogenous TCR Jδ segment with an unrearranged human TCR Jδ segment, and a replacement of an endogenous TCR δ constant region gene sequence with a human TCR δ constant region gene sequence.

In some embodiments,
(A) the unrearranged TCR Vγ segment comprises a full repertoire of unrearranged human TCR Vγ segments and the unrearranged human TCR Jγ segment comprises a full repertoire of unrearranged human TCR Jγ segments; or
(B) the unrearranged human TCR Vδ segment comprises a full repertoire of unrearranged human TCR Vδ segments, the unrearranged human TCR Dδ segment comprises a full repertoire of unrearranged human TCR Dδ segments, and the unrearranged human TCR Jδ segment comprises a full repertoire of unrearranged human TCR Jδ segments;
(C) (i) the unrearranged TCR Vγ segment comprises a full repertoire of unrearranged human TCR Vγ segments and the unrearranged human TCR Jγ segment comprises a full repertoire of unrearranged human TCR Jγ segments, and
   (ii) the unrearranged human TCR Vδ segment comprises a full repertoire of unrearranged human TCR Vδ segments, the unrearranged human TCR Dδ segment comprises a full repertoire of unrearranged human TCR Dδ segments, and the unrearranged human TCR Jδ segment comprises a full repertoire of unrearranged human TCR Jδ segments.

In some humanized γ and/or δ TCR mouse embodiments:
(I) the germ cells and CD3⁻ T cells comprise:
   (A) at the endogenous TCR γ locus:
      a replacement of all endogenous TCR Vγ segments with a full repertoire of unrearranged human TCR Vγ segments,
      a replacement of all endogenous TCR Jγ segments with a full repertoire of unrearranged human TCR Jγ segments, and
      a replacement of all TCR γ constant region gene sequences with a full repertoire of human TCR γ constant region gene sequences; and
   (B) at the endogenous TCR δ locus:
      a replacement all endogenous TCR Vδ segments with a full repertoire of unrearranged human TCR Vδ segments,
      a replacement of all endogenous TCR Dδ segments with a full repertoire of unrearranged human TCR Dδ segments,
      a replacement of all endogenous TCR Jδ segments with a full repertoire of unrearranged human TCR Jδ segments, and
      a replacement of an endogenous TCR δ constant region gene sequence with a human TCR δ constant region gene sequence; and
   (II) the mouse comprises a CD3⁺ T cell that expresses, on its surface, a functional TCR comprising a human TCR γ polypeptide and a human TCR δ polypeptide.

In some humanized γ and/or δ TCR mouse embodiments: the germ cells and CD3⁻ somatic cells further comprise an unrearranged TCRβ variable region sequence comprising at least one unrearranged human TCR variable region VP segment, at least one unrearranged human TCR variable region DP segment, and at least one unrearranged TCR variable region JP segment, wherein the unrearranged TCR β variable region sequence is operably linked to a TCR β constant region gene sequence, such as a mouse TCR β constant region gene sequence, optionally at an endogenous TCR β locus, wherein the unrearranged human TCR VP segment, the unrearranged human TCR DP segment, and the unrearranged human TCR JP segment are capable of rearranging (or rearrange) in a T cell to form a rearranged human TCR Vβ/Dβ/Jβ variable region gene that is operably linked to the TCR β constant region gene sequence, and wherein the rearranged human TCR Vβ/Dβ/Jβ variable region gene operably linked to the TCR β constant region gene sequence together encode a TCR β polypeptide comprising a human TCR β variable domain a TCR β constant domain (e.g., a mouse or human TCR β constant domain); and wherein the mouse further comprises a CD3⁺ T cell that expresses, on its surface, a functional TCR comprising the TCR β polypeptide. In some embodiments, the unrearranged TCRβ variable region sequence comprises a mouse TCRB non-coding sequence.

In some humanized γ and/or δ TCR mouse embodiments:
(I) the germ cells and CD3⁻ somatic cells comprise:
   (A) at the endogenous TCR γ locus:
      a replacement of all endogenous TCR Vγ segments with a full repertoire of unrearranged human TCR Vγ segments,
      a replacement of all endogenous TCR Jγ segments with a full repertoire of unrearranged human TCR Jγ segments, and
      a replacement of all TCR γ constant region gene sequences with a full repertoire of human TCR γ constant region gene sequences; and
   (B) at the endogenous TCR δ locus:
      a replacement of all endogenous TCR Vδ segments with a full repertoire of unrearranged human TCR Vδ segments,
      a replacement of all endogenous TCR Dδ segments with a full repertoire of unrearranged human TCR Dδ segments,
      a replacement of all endogenous TCR Jδ segments with a full repertoire of unrearranged human TCR Jδ segments, and
      a replacement of an endogenous TCR δ constant region gene sequence with a human TCR δ constant region gene sequence; and
   (C) at the endogenous TCR α locus:
      a replacement of all endogenous TCR Vα segments with a full repertoire of unrearranged human TCR Vα segments, and
      a replacement of all endogenous TCR Jα segments with a full repertoire of unrearranged human TCR Jα segments, and
   (D) at the endogenous TCR β locus:
      a replacement of all endogenous TCR VP segments with a full repertoire of unrearranged human TCR VP segments,
      a replacement of all endogenous TCR DP segments with a full repertoire of unrearranged human TCR DP segments, and
      a replacement of all endogenous TCR JP segments with a full repertoire of unrearranged human TCR JP segments; and
(II) the mouse further comprises a CD3⁺ T cell that expresses, on its surface, a functional human TCR comprising a human TCR γ polypeptide and a human TCR δ polypeptide and a CD3⁺ T cell that expresses, on its surface, a human or a human or humanized TCR comprising a human or humanized TCR α polypeptide and a human or humanized TCR β polypeptide.

In some humanized γ and/or δ TCR mouse embodiments:
(I) the germ cells and CD3⁻ somatic cells comprise:
   (A) at the endogenous TCR γ locus:
      a replacement of all endogenous TCR Vγ segments with a full repertoire of unrearranged human TCR Vγ segments,
      a replacement of all endogenous TCR Jγ segments with a full repertoire of unrearranged human TCR Jγ segments, and
      a replacement of all TCR γ constant region gene sequences with a full repertoire of human TCR γ constant region gene sequences; and
   (B) at the endogenous TCR δ locus:
      a replacement all endogenous TCR Vδ segments with a full repertoire of unrearranged human TCR Vδ segments,
      a replacement of all endogenous TCR Dδ segments with a full repertoire of unrearranged human TCR Dδ segments,
      a replacement of all endogenous TCR Jδ segments with a full repertoire of unrearranged human TCR Jδ segments, and
      a replacement of an endogenous TCR δ constant region gene sequence with a human TCR δ constant region gene sequence; and
   (C) at the endogenous TCR α locus:
      a replacement of all endogenous TCR Vα segments with a full repertoire of unrearranged human TCR Vα segments, and
      a replacement of all endogenous TCR Jα segments with a full repertoire of unrearranged human TCR Jα segments;
   (D) at the endogenous TCR β locus:
      a replacement of all endogenous TCR Vβ segments with a full repertoire of unrearranged human TCR Vβ segments,
      a replacement of all endogenous TCR DP segments with a full repertoire of unrearranged human TCR DP segments,
      a replacement of all endogenous TCR Jβ segments with a full repertoire of unrearranged human TCR Jβ segments; and
   (E) a first nucleotide sequence encoding a chimeric human/mouse CD4 co-receptor that comprises D1, D2 and D3 domains of a human CD4 polypeptide operably linked to D4, transmembrane and cytoplasmic domains of a mouse CD4 polypeptide; and
   (F) a second nucleotide sequence encoding a chimeric human/mouse CD8α polypeptide and a third nucleotide sequence encoding a chimeric human/mouse CD8β polypeptide,
      wherein the chimeric human/mouse CD8α polypeptide comprises an IgV-like domain of a human CD8α polypeptide operably linked to transmembrane and cytoplasmic domains of a mouse CD8α polypeptide and wherein the chimeric human/mouse CD8β polypeptide comprises an IgV-like domain of a human CD8β polypeptide operably linked to transmembrane and cytoplasmic domains of a mouse CD8β polypeptide; and
   (G) a first nucleic acid sequence encoding a chimeric human/mouse MHC II α polypeptide and a second nucleic acid sequence encoding a chimeric human/mouse MHC II β polypeptide,
      wherein the chimeric human/mouse MHC II α polypeptide comprises α1 and α2 domains of a human HLA class II α polypeptide operably linked to transmembrane and cytoplasmic domains of a mouse MHC II α polypeptide and
      wherein the chimeric human/mouse MHC II β polypeptide comprises β1 and β2 domains of a human HLA class II β polypeptide operably linked to transmembrane and cytoplasmic domains of a mouse MHC II β polypeptide;
   (H) a third nucleic acid sequence encoding a chimeric human/mouse MHC I polypeptide comprising α1, α2, and α3 domains of a human HLA class I polypeptide operably linked to transmembrane and cytoplasmic domains of a mouse MHC class I polypeptide; and
   (I) a polynucleotide encoding a human or humanized β2 microglobulin polypeptide and comprising the nucleotide sequence set forth in exon 1 of a mouse β2 microglobulin gene operably linked to the nucleotide sequence set forth in exon 2, exon 3, and exon 4 of a human β2 microglobulin gene,
(II) wherein the mouse further comprises a CD3⁺ T cell that expresses, on its surface, a functional human TCR comprising a human TCR γ polypeptide and a human TCR δ polypeptide and a CD3⁺ T cell that expresses, on its surface, a functional human or humanized TCR comprising a human or humanized TCR α polypeptide and a human or humanized TCR polypeptide, and optionally
wherein the mouse expresses:
   (e) the chimeric human/mouse CD4 co-receptor,
   (f) a chimeric CD8 co-receptor comprising the chimeric human/mouse CD8α polypeptide and the chimeric human/mouse CD8β polypeptide,
   (h) a chimeric MHC II complex comprising the chimeric human/mouse MHC II α polypeptide and the chimeric human/mouse MHC II R polypeptide, wherein the chimeric MHC II complex is capable of binding the chimeric human/mouse CD4 co-receptor,
   (i) the chimeric human/mouse MHC I polypeptide, wherein the chimeric MHC I polypeptide is capable of binding the chimeric CD8 co-receptor, and
   (j) the human or humanized β2 microglobulin polypeptide.

In some humanized γ and/or δ TCR mouse embodiments, the germ cells and CD3⁻ somatic cells each comprises a human CTCF binding element upstream of a TCR γ locus, upstream of a TCR α locus, or a first human CTCF binding element upstream of a TCR γ locus and a second human CTCF binding element upstream of a TCR α locus. In some embodiments, the germ cells and somatic cells each comprise a human CTCF binding element upstream a TCR α locus.

In some humanized γ and/or δ TCR mouse embodiments, the mouse comprises γ/δ T cells in its thymus, its spleen, its skin, and/or its gut mucosa, wherein the γ/δ T cells may comprise a human TCR γ polypeptide and a human TCR δ polypeptide. In some γ and/or δ TCR mouse embodiments as described herein; optionally wherein the mouse further comprises human or humanized TCRα and 13, MHC I, MHC II α and 13, CD4, CD8α and β, and/or β2M loci; the mouse may comprise a population of CD45+CD3+ T cells in its spleen, thymus, mesenteric lymph nodes (MLN), skin, gut mucosa, and/or among intraepithelial lymphocytes (IEL) isolated from its colon or small intestine, wherein a percentage of the population of CD45+CD3+ T cells expresses a human γ/δ TCR. In some embodiments, the percentage of splenic, thymic, MLN, skin, gut mucosal, and/or IEL CD45+CD3+ T cells that express human γ/δ TCR in a humanized γ and/or δ TCR mouse embodiment as described herein is comparable (e.g., not significantly different, wherein any difference is not statistically significant, within 10 percentage points of each other, etc.) to the percentage of CD45+CD3+ T cells that express murine γ/δ TCR in a wildtype mouse. In some embodiments, the percentage of splenic, thymic, MLN, and/or IEL CD45+CD3+ T cells that express human γ/δ TCR in a γ and/or δ TCR mouse embodiment as described herein is greater than (e.g., 1.5-fold to 3-fold) the percentage of CD45+CD3+ T cells that express murine γ/δ TCR in a wildtype mouse.

In some humanized γ and/or δ TCR embodiments, the human TCR γ polypeptide is derived from a human TRGV2 gene segment, a human TRGV3 gene segment, a human TRGV4 gene segment, a human TRGV5 gene segment, a human TRGV8 gene segment, a human TRGV9 gene segment, a human TRGV10 gene segment, or a human TRGV11 gene segment. In some embodiments, the human TCR γ polypeptide is derived from a human TRGJ1 gene segment, a human TRGJP gene segment, a human TRGJP1 gene segment, a human TCRGJ2 gene segment, or a human TRGJP2 gene segment. In some embodiments, the human TCR δ polypeptide is derived from a human TRDV1 gene segment, a human TRAV17 gene segment, a human TRAV19 gene segment, a human TRAV21 gene segment, a human TRAV21 gene segment, a human TRAV26-2 gene segment, a human TRAV29/TRDV5 gene segment, a human TRAV31 gene segment, a human TRAV38-2/DV8 gene segment, a human TRAV39 gene segment, a human TRAV40 gene segment, a human TRAV41 gene segment, a human TRDV2 gene segment, or a human TRDV3 gene segment. In some embodiments, the human TCR δ polypeptide is derived from a human TRDJ1 gene segment, a human TRDJ2 gene segment, a human TRDJ3 gene segment, or a human TRDJ4 gene segment.

Also described herein is a mouse embryonic stem (ES) cell or germ cell comprising an unrearranged TCR γ variable region sequence comprising an unrearranged human TCR Vγ segment and an unrearranged human TCR Jγ segment, wherein the unrearranged TCR γ variable region sequence is operably linked to a human TCRγ constant region gene sequence, optionally at an endogenous TCRγ locus (e.g., a human TCRγ constant region gene sequence at an endogenous TCRγ locus, wherein a nucleotide sequence comprising an endogenous TCRγ constant region gene sequence (e.g., an endogenous Trgc1 constant region gene sequence, an endogenous Trgc2 constant region gene sequence, an endogenous Trgc3 constant region gene sequence, and/or an endogenous Trgc4 constant region gene sequence) is replaced with a nucleotide sequence comprising a human TCRγ constant region gene sequence, (e.g., a human TRGC1 constant region sequence and/or a human TRGC2 constant region sequence). In some embodiments, the ES cell or germ cell further comprises an unrearranged T cell receptor (TCR) δ variable region sequence comprising an unrearranged human TCR Vδ segment, an unrearranged human TCR Dδ segment, and an unrearranged human TCR Jδ segment, wherein the unrearranged TCR δ variable region sequence is operably linked to a human TCR δ constant region gene sequence, optionally at an endogenous TCR δ locus. In some embodiments, the ES cell or germ cell further comprises an unrearranged human TCR Vα segment upstream of the unrearranged TCR δ variable region sequence and the human TCR δ constant region gene sequence.

Also described herein is a mouse ES cell or germ cell comprising from 5′ to 3′:
an unrearranged human TCR Vα segment and
an unrearranged TCR δ variable region sequence comprising an unrearranged human TCR Vδ segment, an unrearranged TCR Dδ, and an unrearranged human TCR Jδ segment,
wherein the unrearranged TCR δ variable region sequence is operably linked to a human TCR δ constant region gene sequence, optionally at an endogenous TCR δ locus.

In some embodiments, the ES cell or germ cell comprises a replacement at an endogenous TCR Vα segment with the unrearranged human TCR Vα segment and a replacement of an endogenous TCR Jα segment with an unrearranged human TCR Jα segment, wherein the unrearranged human TCR Vα segment and unrearranged human TCR Jα segment are operably linked to each other and a TCR α constant region gene sequence (e.g., a mouse TCR α constant region gene sequence (optionally at an endogenous TCR α locus) or a human TCR α constant region gene sequence). In some embodiments, the mouse ES cell or germ cell comprises a replacement of all endogenous TCR Vα segments with a full repertoire of unrearranged human TCR Vα segments and a replacement of all endogenous TCR Jα segments with a full repertoire of unrearranged human TCR Jα segments, wherein the full repertoire of unrearranged human TCR Vα segments and full repertoire of unrearranged human TCR Jα segments are operably linked to each other and a mouse TCR α constant region gene sequence at an endogenous TCR α locus.

In some embodiments, the mouse ES cell or germ cell comprises:
(A) a replacement of an endogenous TCR Vγ segment with an unrearranged human TCR Vγ segment, a replacement of an endogenous TCR Jγ segment with an unrearranged human TCR Jγ segment, and a replacement of an endogenous TCR γ constant region gene sequence with an unrearranged human TCR γ constant region gene sequence; or
(B) a replacement of an endogenous TCR Vδ segment with an unrearranged human TCR Vδ segment, a replacement of an endogenous TCR Dδ segment with an unrearranged human TCR Dδ segment, a replacement of an endogenous TCR Jδ segment with an unrearranged human TCR Jδ segment, and a replacement of an endogenous TCR δ constant region gene sequence with a human TCR δ constant region gene sequence; or
(C) (i) a replacement of an endogenous TCR Vγ segment with an unrearranged human TCR Vγ segment, a replacement of an endogenous TCR Jγ segment with an unrearranged human TCR Jγ segment, and a replacement of an endogenous TCR γ constant region gene sequence with a human TCR γ constant region gene sequence, and
   (ii) a replacement of an endogenous TCR Vδ segment with an unrearranged human TCR Vδ segment, a replacement of an endogenous TCR Dδ segment with an unrearranged human TCR Dδ segment, a replacement of an endogenous TCR Jδ segment with an unrearranged human TCR Jδ segment, and a replacement of an endogenous TCR δ constant region gene sequence with a human TCR δ constant region gene sequence.

In some ES or germ cell embodiments:
(A) the unrearranged TCR Vγ segment comprises a full repertoire of unrearranged human TCR Vγ segments and the unrearranged human TCR Jγ segment comprises a full repertoire of unrearranged human TCR Jγ segments; or
(B) the unrearranged human TCR Vδ segment comprises a full repertoire of unrearranged human TCR Vδ segments, the unrearranged human TCR Dδ segment comprises a full repertoire of unrearranged human TCR Dδ segments, and the unrearranged human TCR Jδ segment comprises a full repertoire of unrearranged human TCR Jδ segments; or
(C) (i) the unrearranged TCR Vγ segment comprises a full repertoire of unrearranged human TCR Vγ segments and the unrearranged human TCR Jγ segment comprises a full repertoire of unrearranged human TCR Jγ segments, and
   (ii) the unrearranged human TCR Vδ segment comprises a full repertoire of unrearranged human TCR Vδ segments, the unrearranged human TCR Dδ segment comprises a full repertoire of unrearranged human TCR Dδ segments, and the unrearranged human TCR Jδ segment comprises a full repertoire of unrearranged human TCR Jδ segments.

In some embodiments:
(I) the ES cell or germ cell comprises:
   (A) at the endogenous TCR γ locus:
      a replacement of all endogenous TCR Vγ segments with a full repertoire of unrearranged human TCR Vγ segments,
      a replacement of all endogenous TCR Jγ segments with a full repertoire of unrearranged human TCR Jγ segments, and
      a replacement of all endogenous TCR γ constant region gene sequences with a full repertoire of human TCR γ constant region gene sequences; and
   (B) at the endogenous TCR δ locus:
      a replacement all endogenous TCR Vδ segments with a full repertoire of unrearranged human TCR Vδ segments,
      a replacement of all endogenous TCR Dδ segments with a full repertoire of unrearranged human TCR Dδ segments,
      a replacement of all endogenous TCR Jδ segments with a full repertoire of unrearranged human TCR Jδ segments, and
      a replacement of an endogenous TCR δ constant region gene sequence with a human TCR δ constant region gene sequence.
         In some embodiments, an endogenous TCR γ locus as described herein comprises a replacement of an endogenous genomic sequence comprising all endogenous TCR Vγ segments, all endogenous TCR Jγ segments, and all endogenous TCR γ constant region gene sequences with human genomic sequence comprising a full repertoire of unrearranged human TCR Vγ segments, a full repertoire of unrearranged human TCR Jγ segments, and a full repertoire of human TCR γ constant region gene sequences (e.g., hTCRGC1 and hTCRGC2).

In some embodiments, the ES cell or germ cell further comprises an unrearranged TCRβ variable region sequence comprising at least one unrearranged human T cell variable region Vβ segment, at least one unrearranged human T cell variable region DP segment, and at least one unrearranged human T cell variable region JP segment, wherein the unrearranged TCR β variable region sequence is operably linked to a TCR β constant region gene sequence (e.g., a mouse TCR β constant region gene sequence), optionally at an endogenous TCR β locus. In some embodiments, the unrearranged TCRβ variable region sequence comprises a mouse TCRB non-coding sequence.

In some embodiments, an ES cell or germ cell as described comprises:
(A) at the endogenous TCR γ locus:
   a replacement of all endogenous TCR Vγ segments with a full repertoire of unrearranged human TCR Vγ segments,
   a replacement of all endogenous TCR Jγ segments with a full repertoire of unrearranged human TCR Jγ segments, and
   a replacement of all TCR γ constant region gene sequences with a full repertoire of human TCR γ constant region gene sequences; and
(B) at the endogenous TCR δ locus:
   a replacement of all endogenous TCR Vδ segments with a full repertoire of unrearranged human TCR Vδ segments,
   a replacement of all endogenous TCR Dδ segments with a full repertoire of unrearranged human TCR Dδ segments,
   a replacement of all endogenous TCR Jδ segments with a full repertoire of unrearranged human TCR Jδ segments, and
   a replacement of an endogenous TCR δ constant region gene sequence with a human TCR δ constant region gene sequence; and
(C) at the endogenous TCR α locus:
   a replacement of all endogenous TCR V α segments with a full repertoire of unrearranged human TCR Vα segments, and
   a replacement of all endogenous TCR Jα segments with a full repertoire of unrearranged human TCR Jα segments, and
(D) at the endogenous TCR β locus:
   a replacement of all endogenous TCR Vβ segments with a full repertoire of unrearranged human TCR Vβ segments,
   a replacement of all endogenous TCR Dβ segments with a full repertoire of unrearranged human TCR Dβ segments, and
   a replacement of all endogenous TCR Jβ segments with a full repertoire of unrearranged human TCR Jβ segments.

In some embodiments, an ES cell or germ cell as described herein comprises:
(A) at the endogenous TCR γ locus:
   a replacement of all endogenous TCR Vγ segments with a full repertoire of unrearranged human TCR Vγ segments,
   a replacement of all endogenous TCR Jγ segments with a full repertoire of unrearranged human TCR Jγ segments, and
   a replacement of all TCR γ constant region gene sequences with a full repertoire of human TCR γ constant region gene sequences; and
(B) at the endogenous TCR δ locus:
   a replacement of all endogenous TCR Vδ segments with a full repertoire of unrearranged human TCR Vδ segments,
   a replacement of all endogenous TCR Dδ segments with a full repertoire of unrearranged human TCR Dδ segments,
   a replacement of all endogenous TCR Jδ segments with a full repertoire of unrearranged human TCR Jδ segments, and
   a replacement of an endogenous TCR δ constant region gene sequence with a human TCR δ constant region gene sequence; and
(C) at the endogenous TCR α locus:
   a replacement of all endogenous TCR V α segments with a full repertoire of unrearranged human TCR Vα segments, and
   a replacement of all endogenous TCR Jα segments with a full repertoire of unrearranged human TCR Jα segments, and
(D) at the endogenous TCR β locus:
   a replacement of all endogenous TCR Vβ segments with a full repertoire of unrearranged human TCR Vβ segments,
   a replacement of all endogenous TCR Dβ segments with a full repertoire of unrearranged human TCR Dβ segments,
   a replacement of all endogenous TCR Jβ segments with a full repertoire of unrearranged human TCR Jβ segments,
(E) a first nucleotide sequence encoding a chimeric human/mouse CD4 co-receptor that comprises D1, D2 and D3 domains of a human CD4 polypeptide operably linked to D4, transmembrane and cytoplasmic domains of a mouse CD4 polypeptide;
(F) a second nucleotide sequence encoding a chimeric human/mouse CD8α polypeptide and a third nucleotide sequence encoding a chimeric human/mouse CD8β polypeptide,
   wherein the chimeric human/mouse CD8α polypeptide comprises an IgV-like domain of a human CD8α polypeptide operably linked to transmembrane and cytoplasmic domains of a mouse CD8α polypeptide and wherein the chimeric human/mouse CD8β polypeptide comprises an IgV-like domain of a human CD8β polypeptide operably linked to transmembrane and cytoplasmic domains of a mouse CD8β polypeptide;
(G) a first nucleic acid sequence encoding a chimeric human/mouse MHC II α polypeptide and a second nucleic acid sequence encoding a chimeric human/mouse MHC II β polypeptide,
   wherein the chimeric human/mouse MHC II α polypeptide comprises α1 and α2 domains of a human HLA class II α polypeptide operably linked to transmembrane and cytoplasmic domains of a mouse MHC II α polypeptide and wherein the chimeric human/mouse MHC II β polypeptide comprises β1 and β2 domains of a human HLA class II β polypeptide operably linked to transmembrane and cytoplasmic domains of a mouse MHC II β polypeptide;
(H) a third nucleic acid sequence encoding a chimeric human/mouse MHC I polypeptide comprising α1, α2, and α3 domains of a human HLA class I polypeptide operably linked to transmembrane and cytoplasmic domains of a mouse MHC class I polypeptide; and
(I) a polynucleotide encoding a human or humanized β2 microglobulin polypeptide and comprising the nucleotide sequence set forth in exon 1 of a mouse β2 microglobulin gene operably linked to the nucleotide sequence set forth in exon 2, exon 3, and exon 4 of a human β2 microglobulin gene.

In some ES cell or germ cell embodiments, the ES cell or germ cell further comprises a human CTCF binding element upstream of a TCR γ locus, upstream of a TCR α locus, or a first human CTCF binding element upstream of a TCR γ locus and a second human CTCF binding element upstream of a TCR α locus. In some embodiments, the mouse ES cell or germ cell further comprises a human CTCF binding element upstream a TCR α locus. In some embodiments, the mouse ES cell or germ cell comprises a human nucleotide sequence set forth at chr7:38383439-38230960 (GRCh38 coordinates) and/or a human nucleotide sequence set forth at Chr14:22421820-22464666 (GRCh38 coordinates).

Also described herein are targeting vectors, e.g., targeting vectors comprising a 5′ mouse homology arm and a 3′ mouse homology arm; from 5′ to 3′:
(a) a 5′ mouse homology arm,
(b) a human nucleotide sequence set forth at chr7:38383439-38230960 (GRCh38 coordinates), and
(c) a 3′ mouse homology arm.

Also described is a targeting vector comprising (i) a selection cassette and (ii) a human nucleotide sequence set forth at Chr14:22421820-22464666 (GRCh38 coordinates).

In some methods described herein, the methods may comprise immunizing a genetically modified mouse as described herein with an antigen of interest, allowing said mouse to mount an immune response to the antigen of interest, and obtaining therefrom a nucleic acid sequence(s) encoding a human TCR variable domain that binds the antigen of interest, e.g., a nucleic acid sequence encoding a human TCR γ polypeptide (or variable domain thereof) of a TCR that binds the antigen of interest, a nucleic acid sequence(s) encoding a human TCR δ polypeptide (or variable domain thereof) of a TCR that binds the antigen of interest, a nucleic acid sequence(s) encoding a human TCR α/δ polypeptide (or variable domain thereof) that binds the antigen of interest, wherein the human TCR α/δ polypeptide is encoded by a human rearranged TCR Vα/Dδ/Jδ variable region gene operably linked to a human TCR δ constant region gene sequence, a nucleic acid sequence encoding a human TCR α polypeptide (or variable domain thereof) of a TCR that binds the antigen of interest, a nucleic acid sequence(s) encoding a human TCR β polypeptide (or variable domain thereof) of a TCR that binds the antigen of interest, and any combination thereof.

Also described herein is a method for making a human therapeutic (or human TCR protein), the method comprising immunizing a genetically modified mouse as described herein with an antigen of interest, allowing the mouse to mount an immune response, obtaining, from the mouse, a T cell reactive to the antigen of interest from the mouse, obtaining, from the T cell, a TCR (e.g., a γδ TCR, an α/δγ TCR, an αβ TCR) that binds the antigen of interest and/or a nucleic acid sequence(s) encoding the TCR or encoding the variable domain(s) of the TCR, wherein the TCR comprises a human TCR variable domain, and optionally employing the human TCR variable domain in a human therapeutic. In some embodiments, the human therapeutic (or human TCR protein) is a soluble T cell receptor. In some embodiments, the human therapeutic (or human TCR protein) is a single chain TCR. In some embodiments, the human therapeutic (or human TCR protein) is an scTv. In some embodiments, the soluble T cell receptor is fused to a moiety that can kill an infected or cancer cell (e.g., a cytotoxic molecule (e.g., a chemotherapeutic), toxin, radionuclide, prodrug, or antibody), an immunomodulatory molecule (e.g., a cytokine or a chemokine), and/or an immune inhibitory molecule (e.g., a molecule that inhibits a T cell from killing other cells harboring an antigen recognized by the T cell).

Also described herein is a host cell comprising a nucleic acid molecule made according to any of the methods described herein.

Described herein is a method of making a genetically modified mouse or mouse ES cell, comprising modifying the genome of the mouse or mouse ES cell to comprise:
(a) an unrearranged TCR γ variable region sequence comprising an unrearranged human TCR Vγ segment and an unrearranged human TCR Jγ segment,
   wherein the unrearranged TCR γ variable region sequence is operably linked to a human TCRγ constant region gene sequence, optionally wherein a human TCRγ constant region gene sequence at an endogenous TCRγ locus, e.g., wherein a nucleotide sequence comprising an endogenous TCRγ constant region gene sequence (e.g., an endogenous Trgc1 constant region gene sequence, an endogenous Trgc2 constant region gene sequence, an endogenous Trgc3 constant region gene sequence, and/or an endogenous Trgc4 constant region gene sequence) is replaced with a nucleotide sequence comprising a human TCRγ constant region gene sequence, (e.g., a human TRGC1 constant region sequence and/or a human TRGC2 constant region sequence),
   wherein the unrearranged human TCR Vγ segment and the unrearranged human TCR Jγ segment are capable of rearranging (or rearrange) in a T cell to form a rearranged human TCR Vγ/Jγ variable region gene that is operably linked to the human TCR γ constant region gene sequence, and
   wherein the rearranged human TCR Vγ/Jγ variable region gene operably linked to the human TCR γ constant region gene sequence together encode a human TCR γ polypeptide; and/or
(b) an unrearranged T cell receptor (TCR) δ variable region sequence comprising an unrearranged human TCR Vδ segment, an unrearranged human TCR Dδ segment, and an unrearranged human TCR Jδ segment,
   wherein the unrearranged TCR δ variable region sequence is operably linked to a human TCR δ constant region gene sequence,
   wherein the unrearranged human TCR Vδ segment, the unrearranged human TCR Dδ segment, and the unrearranged human TCR Jδ segment are capable of rearranging (or rearrange) in a T cell to form a rearranged human TCR Vδ/Dδ/Jδ variable region gene that is operably linked to the human TCR δ constant region gene sequence, and
   wherein the rearranged human TCR Vδ/Dδ/Jδ variable region gene operably linked to the human TCR δ constant region gene sequence together encode a human TCR δ polypeptide.

In some embodiments, modifying comprises:
(a) replacing an endogenous genomic sequence comprising endogenous TCR Vγ and Jγ gene segments and an endogenous TCR Cy gene with a heterologous sequence comprising the unrearranged human TCR Vγ segment and the unrearranged human TCR Jγ segment operably linked with the human TCR γ constant region gene sequence (e.g., replacing an endogenous genomic sequence comprising all endogenous TCR Vγ segments, all endogenous TCR Jγ segments, and all endogenous TCR γ constant region gene sequences with human genomic sequence comprising a full repertoire of unrearranged human TCR Vγ segments, a full repertoire of unrearranged human TCR Jγ segments, and a full repertoire of human TCR γ constant region gene sequences) and/or
(b) replacing an endogenous genomic sequence comprising endogenous TCR Vδ, Dδ, Jδ gene segments and an endogenous TCR Cδ gene with a heterologous sequence comprising the unrearranged human TCR Vδ segment, the unrearranged human TCR Dδ segment, the unrearranged human TCR Jδ segment and the human TCR δ constant region gene sequence.

In some method embodiments:
(a) the endogenous genomic sequence comprising endogenous TCR Vγ and Jγ gene segments and an endogenous TCR Cy gene comprises the full repertoire of endogenous TCR Vγ and Jγ gene segments and endogenous TCR Cy genes, and/or
(b) the endogenous genomic sequence comprising endogenous TCR Vδ, Dδ, Jδ gene segments and an endogenous TCR Cδ gene comprises all endogenous TCR Vδ, Dδ, Jδ gene segments and TCR Cδ genes located between TCR Vα and TCR Jα gene segments.

In some method embodiments:
(a) the heterologous sequence comprising the unrearranged human TCR Vγ segment and the unrearranged human TCR Jγ segment operably linked with the human TCR γ constant region gene sequence comprises a full repertoire of unrearranged TCR Vγ and unrearranged human TCR Jγ segments and all human TCR γ constant region gene sequences (e.g., hTCRGC1 and hTCRGC2), and/or
(b) the heterologous sequence comprising the unrearranged human TCR Vδ segment, the unrearranged human TCR Dδ segment, the unrearranged human TCR Jδ segment and the human TCR δ constant region gene sequence comprises a full repertoire of unrearranged TCR Vδ, unrearranged Dδ, and unrearranged human TCR Jδ segments and a human TCR Cδ gene sequence located between a human TCR Vα gene segment and a human TCR Jα gene segment on chromosome 14 of a human genome.

In some method embodiments, modifying comprises homologous recombination in one or more ES cells such that the heterologous sequence comprising the unrearranged human TCR Vγ segment and the unrearranged human TCR Jγ segment operably linked with the human TCR γ constant region gene sequence; and the heterologous sequence comprising the unrearranged human TCR Vδ segment, the unrearranged human TCR Dδ segment, the unrearranged human TCR Jδ segment, and the human TCR δ constant region gene sequence; are added, in any order, into the genome of the one or more ES cell(s). Some methods further comprise generating a mouse from the one or more ES cells.

In some method embodiments, modifying comprises:
(a) obtaining a first mouse comprising a homozygous replacement of an endogenous genomic sequence comprising endogenous TCR Vγ and Jγ gene segments and an endogenous TCR Cy gene with the heterologous sequence comprising the unrearranged human TCR Vγ segment and the unrearranged human TCR Jγ segment operably linked with the human TCR γ constant region gene sequence,
(b) obtaining a second mouse comprising a homozygous replacement of an endogenous genomic sequence comprising endogenous TCR Vδ, Dδ, Jδ gene segments and an endogenous TCR Cδ gene with the heterologous sequence comprising the unrearranged human TCR Vδ segment, the unrearranged human TCR Dδ segment, the unrearranged human TCR Jδ segment operably linked to the human TCR δ constant region gene sequence, and
(c) breeding the first mouse and the second mouse to obtain a genetically modified mouse,
wherein the genetically modified mouse comprises:
   (i) the replacement of an endogenous genomic sequence comprising endogenous TCR Vγ and Jγ gene segments and an endogenous TCR Cy gene with the heterologous sequence comprising the unrearranged human TCR Vγ segment and the unrearranged human TCR Jγ segment operably linked with the human TCR γ constant region gene sequence, and
   (ii) the replacement of an endogenous genomic sequence comprising endogenous TCR Vδ, Dδ, Jδ gene segments and an endogenous TCR Cδ gene with the heterologous sequence comprising the unrearranged human TCR Vδ segment, the unrearranged human TCR Dδ segment, the unrearranged human TCR Jδ segment operably linked to the human TCR δ constant region gene sequence, and
wherein the genetically modified mouse expresses a human TCR γ polypeptide and a human TCR δ polypeptide.

### BRIEF DESCRIPTION OF THE DRAWINGS

FIG. 1 depicts antigen recognition by different T cell receptor (TCR) embodiments. The left panel shows a non-limiting α/β T cell embodiment interacting with a non-limiting antigen presenting cell embodiment, in which a chimeric α/β TCR recognizes an antigen (grey circle) presented in the context of a chimeric major histocompatibility complex (MHC) I polypeptide that (i) comprises extracellular domains of a human leukocyte antigen (HLA) class I polypeptide and (ii) is associated with a human β2 microglobulin (β2M). The middle panel shows a non-limiting α/β T cell embodiment interacting with a non-limiting professional antigen presenting cell embodiment, in which a chimeric α/β TCR recognizes an antigen (grey circle) presented in the context of a chimeric MHC II that (i) comprises extracellular domains of an HLA class II complex. The right panel shows a non-limiting γ/δ T cell embodiment, which recognizes antigen independent of an MHC. The domains and components of MHC I and MHC II are shown together with their respective co-receptors, CD8 and CD4. Mouse regions are solid and human regions are unfilled/dotted. In various embodiments presented in the figure, the associations depicted can take place in the genetically modified animal (e.g., mouse) described herein.

FIG. 2 depicts (not to scale) a strategy for inserting a human TCRδ locus into the humanized TCRα allele (MAID1771) of a mouse TCRα locus, wherein the TCRδ locus is added downstream of human TCR Vα segments and upstream of the human TCR Jα segments. Also shown is a strategy for inserting a human CTCF binding element upstream of the humanized TCRα locus. Mouse coding sequences are indicated by closed symbols; human coding sequences are indicated by open symbols, unless indicated otherwise. Mouse intergenic or non-coding sequences between coding sequences are depicted by a solid line; human intergenic or non-coding sequences between coding sequences are depicted by unfilled double lines, unless otherwise indicated. MAID refers to modified allele ID number. H=human, TRAV=TCR Vα segment, TRAJ=TCR Jα segment (hTRAJ=human TRAJ), TRAC=TCR Ca domain, TCRD=TCR δ, BHR=bacterial homologous recombination, EP=electroporation, CTCF=CCCTC-binding factor, Loxp-Ub-Hyg=a foxed hygromycin resistance gene under the control of a ubiquitin promoter.

FIG. 3A depicts (not to scale) deletion of all mouse TCRγ V and J segments and TCRγ C genes from a mouse TCRγ locus to form MAID20143 allele. FIG. 3B depicts a strategy for insertion of human TCRγ V and J segments and human TCRγ C genes into the mouse TCRγ locus lacking all mouse TCRγ V and J segments and TCRγ C genes to form the human TCRγ allele (MAID20200). Mouse coding sequences are indicated by closed symbols; human coding sequences are indicated by open symbols, unless otherwise indicated. Mouse intergenic or non-coding sequences between coding sequences are depicted by a solid line; human intergenic or non-coding sequences between coding sequences are depicted by unfilled double lines, unless otherwise indicated. Although not depicted in FIG. 3B, MAID20200 comprises all 15 hTRGV segments, all 5 hTRGJ segments, and both hTRGC genes in germline configuration. MAID refers to modified allele ID number. H=human, TRGV=TCR Vγ segment, TRGJ=TCR Jγ segment, TRGC=TCR Cγ gene, TCRG=TCRγ, BHR=bacterial homologous recombination, EP=electroporation, CTCF=CCCTC-binding factor, Loxp-Hyg-Loxp=a floxed hygromycin resistance gene under the control of a ubiquitin promoter.

FIG. 4 depicts (not to scale) resulting homozygous MAID6981 allele after removal of the selection cassette from MAID6980 and homozygous MAID20201 allele after removal of the selection cassettes from MAID20200. Although not depicted in FIG. 4, MAID20201 comprises all 15 hTRGV segments, all 5 hTRGJ segments, and both hTRGC genes in germline configuration. Mouse coding sequences are indicated by closed symbols; human coding sequences are indicated by open symbols, unless indicated otherwise. Mouse intergenic or non-coding sequences between coding sequences are depicted by a solid line; human intergenic or non-coding sequences between coding sequences are depicted by unfilled double lines, unless otherwise indicated. MAID refers to modified allele ID number. H=human, TRAV=TCR Vα segment, TRAJ=TCR Jα segment (hTRAJ=human TRAJ), TRAC=TCR Cα domain, TCRD=TCRδ locus, enh=enhancer, CTCF=CCCTC-binding factor, Loxp=lox recombination site after Cre recombinase removal of floxed drug cassette.

FIG. 5A depicts representative FACS dot plots of mouse spleen cells from three different wildtype (“WT”), and three mice homozygous for humanized TCRα/δ and γ and wildtype TCR loci (6981 HO 20201HO) stained with anti-human γ/δ (Y-axis) and anti-mouse γ/δ (X-axis) antibodies. FIG. 5B depicts representative FACS plots of mouse thymus cells from three different wildtype (“WT”), and three mice homozygous for humanized TCRα/δ and γ and wildtype TCR β loci (6981 HO 20201HO) stained with anti-human γ/δ (Y axis) and anti-mouse γ/δ (X-axis) antibodies.

FIG. 6A depicts the proportion of TCRd mRNAs (Y-axis) utilizing the indicated human TCRα V and TCRδ V segments in thymic or splenic T cells of mice homozygous for humanized TCRα/δ and γ and wildtype TCR β loci (6981 HO 20201HO), as determined by NGS. FIG. 6B depicts the proportion of TCRd mRNAs (Y axes) utilizing the indicated TCRδ J segments present in thymic or splenic T cells of mice homozygous for humanized TCRα/δ and γ and wildtype TCR β loci (6981 HO 20201HO), as determined by NGS.

FIG. 7A depicts the proportion of TCRg mRNAs (Y-axes) utilizing the indicated human TCRγ V segments present in thymic or splenic T cells of mice homozygous for humanized TCRα/δ and γ and wildtype TCR β loci (6981 HO 20201HO hTCRα). FIG. 7B depicts the proportion of TCRg mRNAs (Y-axes) utilizing the indicated human TCRγ J segments present in thymic or splenic T cells of mice homozygous for humanized TCRα/δ and γ and wildtype TCR loci (6981HO 20201HO hTCRα).

FIG. 8A depicts (not to scale) a progressive strategy for humanization of the mouse TCRα locus, wherein TCRα variable region gene segments are sequentially added upstream of an initial humanization of a deleted mouse locus (MAID1540). Mouse coding sequences are indicated by filled shapes; human coding sequences is indicated by open shapes, unless otherwise indicated. Although not depicted in this figure, each non-coding sequence between each human TRAV and each human TRAJ is human. The remaining intergenic or non-coding sequences are mouse. MAID refers to modified allele ID number. TRAV=TCR Vα segment, TRAJ=TCR Jα segment (hTRAJ=human TRAJ), TRAC=TCR Cα domain, TCRD=TCRδ. FIG. 8B depicts (not to scale) a progressive strategy for humanization of the mouse TCRβ locus, wherein TCRβ variable region gene segments are sequentially added to a deleted mouse TCRβ variable locus. Mouse coding sequences are indicated by filled shapes; human coding sequences are indicated by empty shapes, unless otherwise indicated. Although not depicted in this figure, each non-coding sequence between each human TRBV, between the human D1 and the closest human J, between the human D2 and each human J, and between each J, is human. The remaining intergenic or non-coding sequences are mouse. MAID refers to modified allele ID number. TRBV or TCRBV=TCRβ V segment. FIG. 8C depicts a schematic representation (not to scale) of (a) a TRBDJ1 cluster where the D1 and J1 gene segments are human and the non-coding sequences between them, including RSSs and other intergenic sequences, are mouse, (b) a mouse TRB C1 constant region gene sequence, (c) a TRBDJ2 cluster where the D2 and J2 gene segments are human and the non-coding sequences between them, including RSSs and other intergenic sequences, are mouse, and (d) a mouse TRB C2 constant region gene sequence. Mouse coding sequences are indicated by filled shapes; human coding sequences are indicated by empty shapes. As depicted in this figure, each non-coding sequence between each human TRBD segment and each human TRBJ segment, and between each TRBJ segment, is mouse. A loxP sequence is depicted as an arrow labeled accordingly.

FIG. 9A depicts a schematic representation (not to scale) of the chimeric CD4 locus. Human coding exonic sequences are presented by striped shapes, mouse coding exonic sequences are presented by filled shapes, and non-coding exonic sequences are presented by empty shapes. Immunoglobulin-like domains (Ig), transmembrane (TM), cytoplasmic (CYT) and signal peptide (Signal) coding exons, as well ‘s 3’ untranslated regions (UTR), are indicated. A floxed (loxP) neomycin phosphotransferase (Pgk-neo) cassette is depicted with arrows labeled accordingly. FIG. 9B depicts a schematic representation (not to scale) of the chimeric CD8α and CD8b loci. Human coding exonic sequences are presented by striped shapes, mouse coding exonic sequences are presented by filled shapes, and non-coding exonic sequences are presented by empty shapes. Immunoglobulin-like domains (IgV), transmembrane (TM), cytoplasmic (CYT) and signal peptide (Signal) coding exons, as well as 3′ untranslated regions (UTR), are indicated. Floxed (loxP) hygromycin (Hyg) and neomycin phosphotransferase (Pgk-neo) cassettes are depicted with arrows labeled accordingly.

FIGS. 10A-C provide a schematic representation (not to scale) of exemplary chimeric MHC I and MHC II loci, e.g., chimeric HLA-A2/H-2K locus (FIG. 10A), chimeric HLA-DR2/H-2E locus (FIG. 10B), and humanized β2M locus (FIG. 10C). Unless otherwise indicated, human sequences are depicted as empty shapes and mouse sequences are depicted as filled shapes. The striped shape represents exon 1 of H-2E derived from a different mouse strain than the endogenous locus (see FIG. 11B). Floxed neomycin phosphotransferase cassette(s) are depicted with arrows labeled accordingly.

FIGS. 11A-C depicts a strategy for generating a humanized MHC locus comprising humanized MHC I and MHC II genes. In the particular embodiment depicted in FIG. 11A, the MHC locus of the generated mouse comprises chimeric HLA-A2/H-2K and HLA-DR2/H-2E sequences (H2-H^{+/1666} MHC-II^{+/6112}) and lacks H2-D sequence (H2-D^{+/delete}) and H-2A sequence (the genetic engineering scheme also results in a deletion of H-2A). Large Targeting Vectors (LTVECs) or Cre recombinase construct introduced into ES cells at each stage of humanization are depicted to the right of the arrows. MAID or 4 digit numbers refer to modified allele ID number. FIG. 11B is a schematic diagram (not to scale) of an exemplary HLA-DR2/H-2E large targeting vector. Unless otherwise indicated, human sequences are depicted as empty shapes and mouse sequences are depicted as filled shapes. The striped shape represents exon 1 of H-2E derived from a different mouse strain than the endogenous locus. A floxed hygromycin cassette is depicted as an arrow labeled accordingly. FIG. 11C is a schematic representation (not to scale) of exemplary genotypes of chimeric human/mouse MHC loci (** represents H-2L gene that is not present in all mouse strains, e.g., is not present in C57BL/6 or 129 mouse strains), where endogenous mouse H-2K and H-2E loci are respectively replaced by chimeric human/mouse HLA-A2/H-2K and HLA-DR2/H-2E loci (striped shapes), H-2A and H-2D loci were deleted (empty shapes outlined with dotted lines), and remaining loci are endogenous mouse genes (solid shapes outlined with solid lines).

FIGS. 12A-12D depict the presence of γδ T cells in spleen, thymus, gut tissues, and skin of mice homozygous for humanizations of TCRα/δ, TCRβ, TCRγ, MHC I, MHC II α and β, CD4, CD8α and β, and β2M loci (“VelociT αβγδ mice) or wildtype (WT) mice. FIG. 12A depicts representative FACS dot plots of mouse CD45+CD3+ spleen T cells from two different VelociT αβγδ mice and two different WT mice stained with anti-human γ/δ (Y-axis) and anti-mouse γ/δ (X-axis) antibodies. FIG. 12B depicts representative FACS dot plots of mouse CD45+CD3+ thymus T cells from two different VelociT αβγδ mice and two different WT mice stained with anti-human γ/δ (Y-axis) and anti-mouse γ/δ (X-axis) antibodies. FIG. 12C depicts representative FACS dot plots of CD45+CD3+ T cells from mesenteric lymph nodes (MLN; first column) and intraepithelial lymphocytes (IEL) isolated from the colon (second column) and small intestine (third column) from two different VelociT αβγδ mice and two different WT mice stained with anti-human γ/δ (Y-axis) and anti-mouse γ/δ (X-axis) antibodies. FIG. 12D depicts representative FACS contour plots of CD45+CD3+ skin epidermis T cells from two different VelociT αβγδ mice and two different WT mice stained with anti-human γ/δ (Y-axis) and anti-mouse γ/δ (X-axis) antibodies.

### DETAILED DESCRIPTION

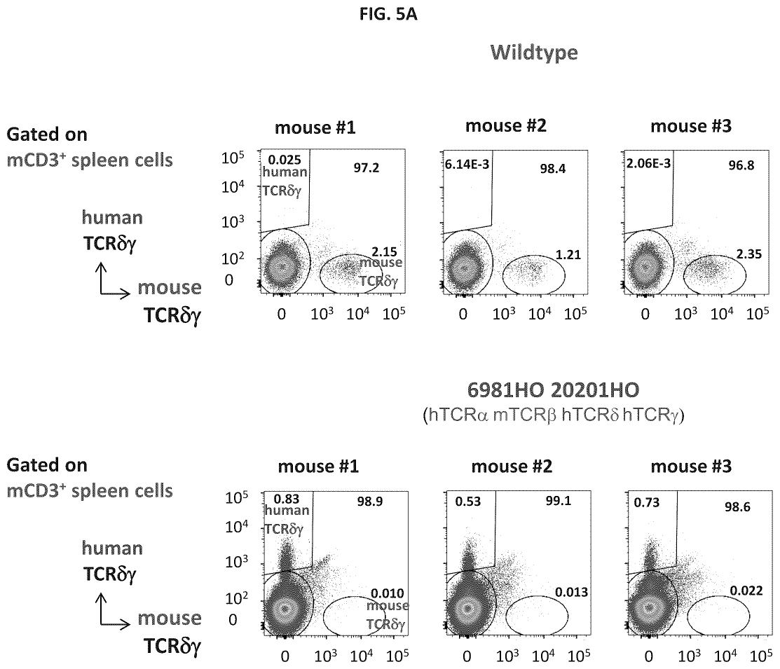

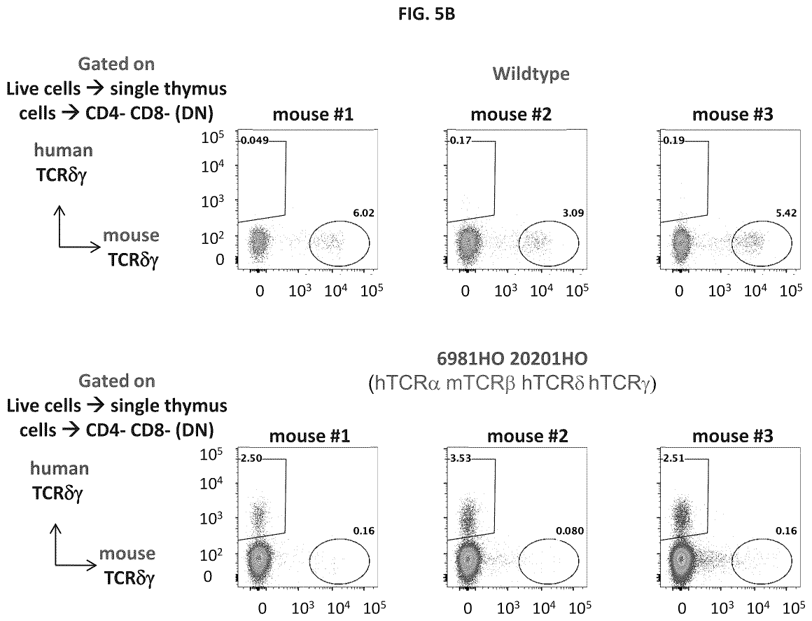

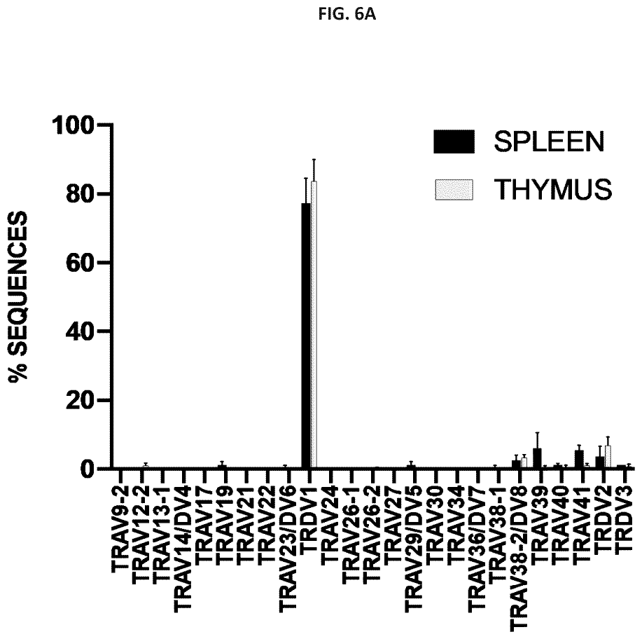

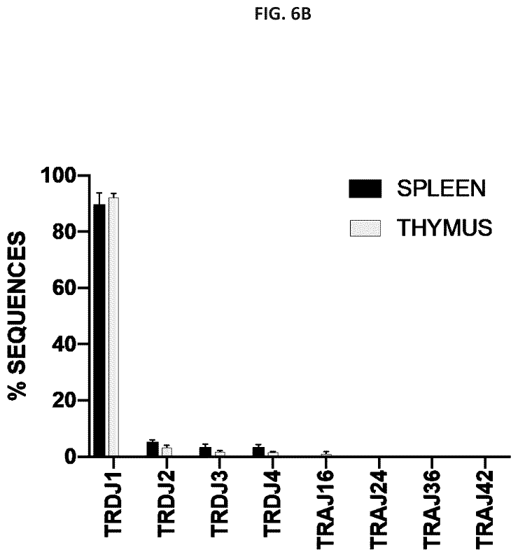

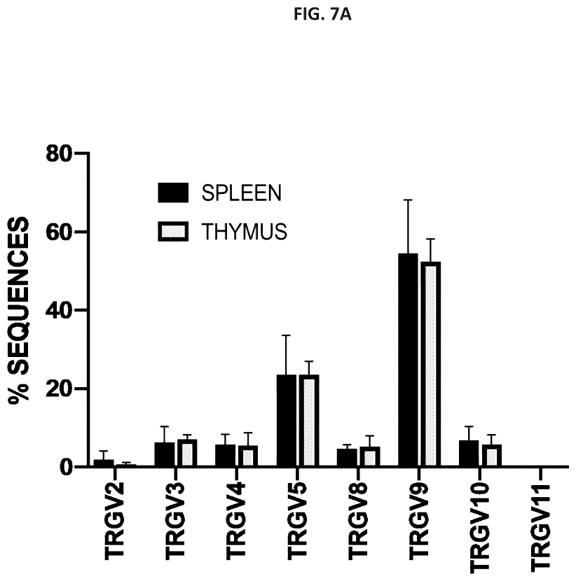

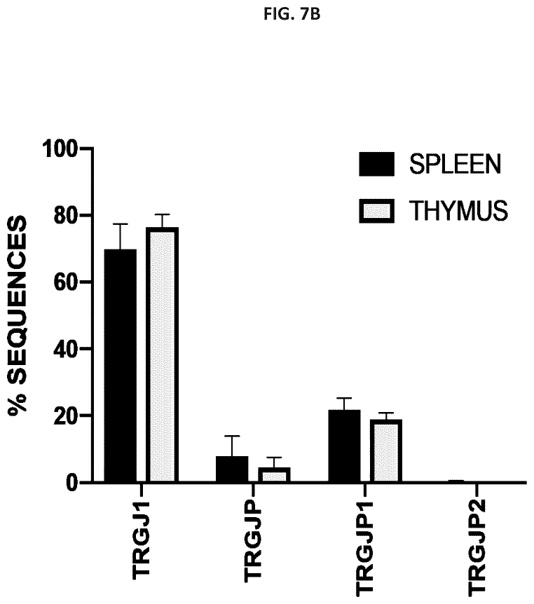

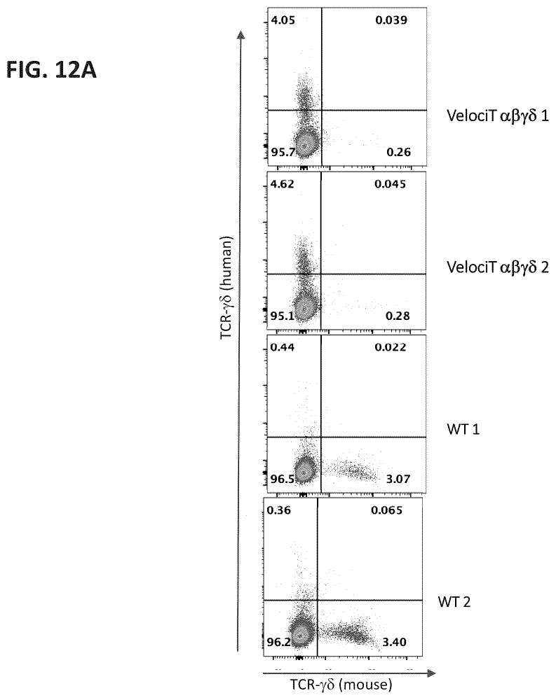

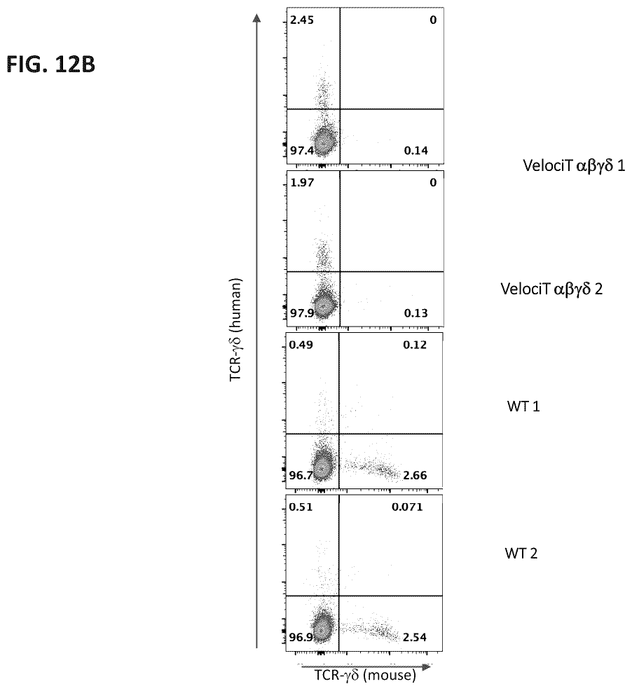

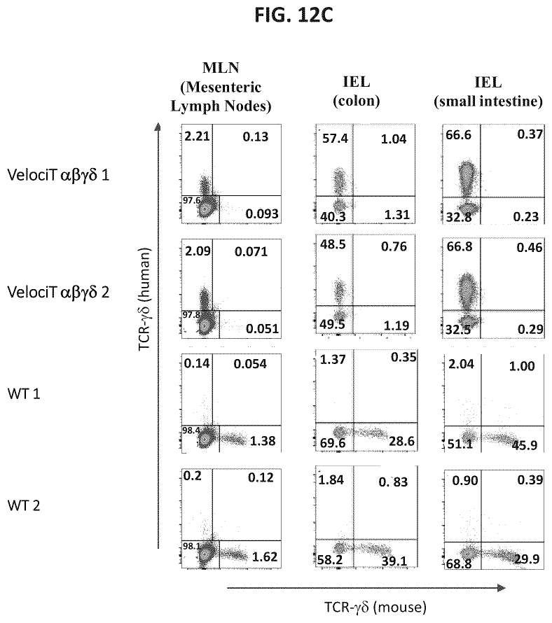

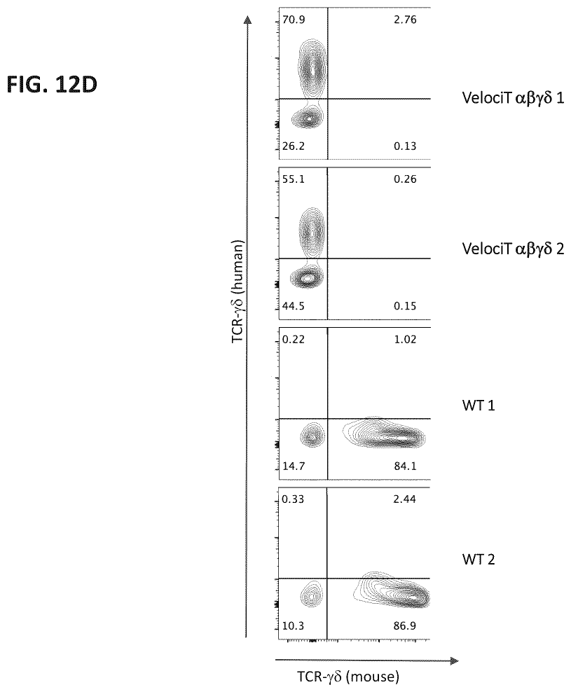

Described herein are non-human animals (e.g., rodents (e.g., rats or mice)) with humanized TRG (encoding TCR-γ polypeptide) and/or TRD (encoding TCR-δ polypeptide) loci, which may lead to potential therapeutics utilizing human γ/δ T cells. As shown herein for such genetically engineered mice, mice with human or humanized TRD (encoding TCR-δ polypeptide), and/or human or TRG (encoding TCR-γ polypeptide) loci comprise human or humanized γ/δ T cells in the thymus and spleen at levels comparable to mice with fully murine components (FIGS. 5A and 5B). These mice also use a diverse repertoire of human TCR γ gene segments and/or human TCR δ gene segments when present (FIGS. 6A, 6B, 7A, and 7B), indicating proper recombination, expression and thymic selection of human or humanized γ/δ TCRs. Also described herein are tissues and cells expressing the human or humanized γ/δ TCRs; methods of using the animals, tissues and cells expressing the human or humanized γ/δ TCRs; methods of making the animals; and embryonic stem (ES) cells and germ cells that may be used in producing such animals. Moreover, described herein are non-human animals (e.g., mice) comprising the human or humanized TRD and TRG loci, along with human or humanized TRA and TRAB loci and human or humanized loci of other components of T cell immunity, including loci of TCR co-receptors (CD4 and CD8) and MHC. Such mice will therefore bear full humanization of both α/β and γ/δ T cell lineages, in contrast to the published “VelociT,” mouse (Moore, M. et al. Sci Immunol 6 (2021); supra) which had α/β but no γ/δ T cell humanization. VelociT αβγδ mice homozygous for one or more unrearranged human or humanized TCRγ, TCRδ, TCRα, and/or TCRβ loci, one or more human or humanized co-receptor loci, and/or one or more MHC loci comprise human or humanized γ/δ CD45+CD3+ T cells in the thymus, spleen, skin, mesenteric lymph nodes (MLN), and among intraepithelial lymphocytes (IEL) isolated from the colon or small intestine, at levels comparable to or greater than mice with fully murine components (FIGS. 12A-12D). Tissues and cells expressing human or humanized α/β and γ/δ T cell lineages, human or humanized T cell co-receptors and MHC; methods of using the animals with a such humanization, methods of making the animals; and embryonic stem (ES) cells and germ cells that may be used in producing such animals are also described.

The terms “chain” and “polypeptides” encompasses contiguous amino acids covalently linked and having a specific amino acid sequence; such terms may be used interchangeably herein. Generally, a T cell receptor comprises two TCR chains/polypeptides (e.g., a TCR γ polypeptide associated with a TCR δ polypeptide, a TCR γ polypeptide associated with a hybrid TCR α/δ polypeptide that comprises a hybrid TCR α/δ variable domain and a TCR δ constant domain, or a TCR α polypeptide associated with a TCR β polypeptide).

One skilled in the art would understand that in addition to the nucleic acid residues encoding TCR variable region gene segments (e.g., TCR V, D, and J gene segments) and/or the optional humanized T cell co-receptor polypeptides, humanized MHC polypeptides, and β2 microglobulin described herein, due to the degeneracy of the genetic code, other nucleic acids may encode the polypeptides of the invention. Therefore, in addition to a genetically modified non-human animal that comprises in its genome an unrearranged human TCRγ variable region gene segment, a human TCRγ constant region gene sequence, an unrearranged human TCR variable region gene segment, and/or a human TCRδ constant region gene sequence (and optionally an unrearranged human TCRα variable region gene segment, an unrearranged human TCRβ variable region gene segment; a nucleotide sequence encoding a humanized T cell co-receptor polypeptide, e.g., CD4 or CD8 polypeptide; and/or a nucleic acid sequence(s) encoding a humanized MHC polypeptide(s)); also provided is a non-human animal whose genome comprises such gene segments, constant region gene sequences, and optional nucleotide sequence(s) encoding a humanized T cell co-receptor polypeptide (e.g., CD4 or CD8 polypeptide); and optionally nucleic acid sequences encoding a humanized MHC polypeptide capable of associating with the humanized T cell co-receptor polypeptide, which differs from that described herein due to the degeneracy of the genetic code or which differs to encode a conservative amino acid substitution.

Also described herein is a genetically modified non-human animal whose genome comprises (e.g., at an endogenous locus) a nucleotide sequence encoding a non-variable amino acid sequence or polypeptide, (e.g., TCR framework regions, TCR constant domains, CD4 or CD8 polypeptide, MHC polypeptides, etc.), wherein the non-variable amino acid sequence or polypeptide comprises conservative amino acid substitutions of the amino acid sequence(s) described herein.

A conservative amino acid substitution includes substitution of an amino acid residue by another amino acid residue having a side chain R group with similar chemical properties (e.g., charge or hydrophobicity). Conservative amino acid substitutions may be achieved by modifying a nucleotide sequence to introduce a nucleotide change that will encode the conservative substitution. In general, a conservative amino acid substitution will not substantially change the functional properties of interest of a protein, for example, the ability of CD4 or CD8 to associate with, e.g., bind to MHC II or MHC I, respectively. Examples of groups of amino acids that have side chains with similar chemical properties include aliphatic side chains such as glycine, alanine, valine, leucine, and isoleucine; aliphatic-hydroxyl side chains such as serine and threonine; amide-containing side chains such as asparagine and glutamine; aromatic side chains such as phenylalanine, tyrosine, and tryptophan; basic side chains such as lysine, arginine, and histidine; acidic side chains such as aspartic acid and glutamic acid; and, sulfur-containing side chains such as cysteine and methionine. Conservative amino acids substitution groups include, for example, valine/leucine/isoleucine, phenylalanine/tyrosine, lysine/arginine, alanine/valine, glutamate/aspartate, and asparagine/glutamine. In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet et al. ((1992) Exhaustive Matching of the Entire Protein Sequence Database, Science 256:1443-45), hereby incorporated by reference. In some embodiments, the substitution is a moderately conservative substitution wherein the substitution has a nonnegative value in the PAM250 log-likelihood matrix.

The identity of a sequence may be determined by a number of different algorithms known in the art that can be used to measure nucleotide and/or amino acid sequence identity. In some embodiments described herein, identities are determined using a ClustalW v. 1.83 (slow) alignment employing an open gap penalty of 10.0, an extend gap penalty of 0.1, and using a Gonnet similarity matrix (MacVector™ 10.0.2, MacVector Inc., 2008). The length of the sequences compared with respect to identity of sequences will depend upon the particular sequences. In various embodiments, identity is determined by comparing the sequence of a mature protein from its N-terminal to its C-terminal. In various embodiments when comparing a chimeric human/non-human sequence to a human sequence, the human portion of the chimeric human/non-human sequence (but not the non-human portion) is used in making a comparison for the purpose of ascertaining a level of identity between a human sequence and a human portion of a chimeric human/non-human sequence (e.g., comparing a human ectodomain of a chimeric human/mouse protein to a human ectodomain of a human protein).

The terms “homology” or “homologous” in reference to sequences, e.g., nucleotide or amino acid sequences, means two sequences which, upon optimal alignment and comparison, are identical in, e.g., at least about 75% of nucleotides or amino acids, e.g., at least about 80% of nucleotides or amino acids, e.g., at least about 90-95% nucleotides or amino acids, e.g., greater than 97% nucleotides or amino acids. One skilled in the art would understand that, for optimal gene targeting, the targeting construct should contain arms homologous to endogenous DNA sequences (i.e., “homology arms”); thus, homologous recombination can occur between the targeting construct and the targeted endogenous sequence.

The term “operably linked” refers to a juxtaposition wherein the components so described are in a relationship permitting them to function in their intended manner. As such, a nucleic acid sequence encoding a protein may be operably linked to regulatory sequences (e.g., promoter, enhancer, silencer sequence, etc.) so as to retain proper transcriptional regulation. In addition, various portions of a chimeric or humanized protein of the invention may be operably linked to retain proper folding, processing, targeting, expression, and other functional properties of the protein in the cell. Unless stated otherwise, various domains of the chimeric or humanized proteins of the invention are operably linked to each other.

The term “replacement” in reference to gene replacement refers to placing exogenous genetic material at an endogenous genetic locus, thereby replacing all or a portion of the endogenous gene with an orthologous or homologous nucleic acid sequence. As demonstrated in the Examples below, in one embodiment, endogenous gene segments or constant region gene sequence(s) of a TCR γ locus were replaced by (orthologous) human TCR γ gene segments or human TCR γ constant region gene sequence(s), respectively, and/or endogenous gene segments or constant region gene sequence of a TCR δ locus were replaced by (orthologous) human TCR δ gene segments or human TCR δ constant region gene sequence, respectively.

“Functional” as used herein, e.g., in reference to a functional polypeptide, refers to a polypeptide that retains at least one biological activity normally associated with the native protein. For example, in some embodiments of the invention, a replacement at an endogenous non-human animal TCR locus of a non-human animal TCR gene with a human TCR gene results in a locus that may fail to express a functional endogenous TCR polypeptide, but that may be able to express a functional human TCR polypeptide. As a non-limiting example, respectively replacing an endogenous non-human TCR γ gene and/or an endogenous non-human TCR δ gene with a human TCR γ gene and/or a human TCR δ gene in a non-human animal may result in the non-human animal expressing a functional TCR (e.g., a TCR capable of binding antigen, transmitting activation signals, and/or initiate a CD3⁺ T cell mediated immune response) that comprises a human TCR γ polypeptide and/or human TCR δ polypeptide, respectively.

### Non-Human Animals, Tissues, Cells and Macromolecules

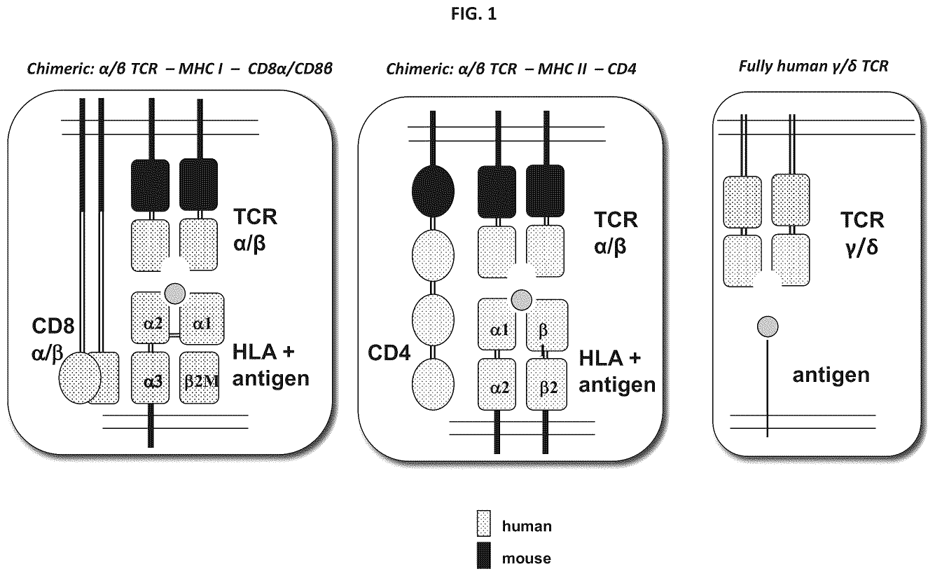

Vertebrate adaptive immune systems harbor two T cell lineages that utilize diverse antigen receptors arising from somatic recombination of DNA segments encoding their antigen recognition—or variable—domains. T cells bind epitopes on small antigenic determinants on the surface of antigen-presenting cells that are associated with a major histocompatibility complex (MHC; in mice) or human leukocyte antigen (HLA; in humans) complex. T cells bind these epitopes through a T cell receptor (TCR) complex on the surface of the T cell that predominantly recognize peptide antigens presented by the major histocompatibility complex (MHC) molecules. Non-limiting and exemplary interactions of α/β TCRs with MHC class I (presenting antigen to CD8+ T cells) and MHC class II (presenting antigen to CD4+ T cells) molecules, or γ/δ TCR with antigen according to embodiments described herein are shown in FIG. 1 (closed symbols represent non-human sequences; striped symbols represent human sequences).

T cell receptors are heterodimeric structures composed of two types of chains: an α (alpha) and β (beta) chain, or a γ (gamma) and δ (delta) chain. Alpha/beta (α/β) T cells are the most abundant T cell lineage. The α chain is encoded by the nucleic acid sequence located within the α locus (on human or mouse chromosome 14), which also encompasses the entire δ locus that encodes the δ chain; the β chain is encoded by the nucleic acid sequence located within the β locus (on mouse chromosome 6 or human chromosome 7); and the γ chain is encoded by the nucleic acid sequence located within the γ locus (on mouse chromosome 13 or human chromosome 7). The majority of T cells has an α/β TCR; while a minority of T cells bears a γ/δ TCR.

T cell receptor γ and δ polypeptides (and similarly α and β polypeptides) are linked to each other via a disulfide bond. Each of the two polypeptides that make up the TCR contains an extracellular domain comprising variable and constant domains, a transmembrane domain, and a cytoplasmic tail (the transmembrane domain and the cytoplasmic tail also being a part of the constant domain). The variable domain of the TCR determines its antigen specificity and comprises three complementary determining regions (CDRs).

The three-dimensional structure of the antigen-recognition site of a T cell receptor looks similar to that of an antibody and primarily comprises three complementarity determining regions (CDR1, CDR2, CDR3) that are flanked by framework regions. The periphery of the antigen binding site comprises CDR1 and CDR2 loops and the center of the antigen-binding site of a TCR is formed by CDR3.

The structural diversity of T-cell receptors is mainly attributable to combinatorial and junctional diversity generated during rearrangement. T cell receptor variable gene loci in the germline DNA (e.g., the DNA that is found in all germ cells contain a number of each of unrearranged TCR variable (V) segments, TCR diversity (D) segments (for TCR β and TCR δ loci), and TCR joining (J) segments. During rearrangement, one of each plurality of V(D)J segments join together during recombination to form a rearranged V/(D)/J variable region gene sequence that is operably linked to a TCR constant region gene sequence in such a way that the variable region gene sequence and TCR constant region gene sequence together encode a TCR polypeptide. The unrearranged TCR V, D, and J gene segments are flanked by recombination signal sequences (RSSs) of 12- or 23-mer spacer length that direct recombination according to the well-established “12/23 rule” of recombination. D genes in the TCRβ and TCRδ loci are flanked by a 12RSS and 23RSS, and their recombination may be controlled by mechanisms in addition to the 12/23 rule. See, e.g., Olaru A., et al. (2005) J. Immunol. 174(10):6220-6226, incorporated herein in its entirety by reference. Any TCR segment operably linked to an RSS has not undergone recombination, and thus, may be considered an “unrearranged” segment. Thus, each “unrearranged” TCR V segment, D segment, or J segment is operably linked to (e.g., associated with, flanked on by one or both sides with, contiguous with, etc.) a recombination signal sequence (RSS), which may be a 12 mer RSS or a 23 mer RSS. In this manner and according to the 12/23 rule: an unrearranged TCR Vα segment is able to rearrange with an unrearranged TCR Jα segment to form a TCR Vα/Jα gene sequence that encodes a TCR α variable domain, an unrearranged TCR Vβ segment is able to rearrange with an unrearranged TCR Dβ segment and an unrearranged TCR Jβ to form a TCR Vβ/Dβ/Jβ gene sequence that encodes a TCR β variable domain, an unrearranged TCR Vγ segment is able to rearrange with an unrearranged TCR Jγ segment to form a TCR Vγ/Jγ gene sequence that encodes a TCR γ variable domain, an unrearranged TCR Vδ segment is able to rearrange with an unrearranged TCR Dδ segment and an unrearranged TCR Jδ segment to form a TCR Vδ/DδJδ gene sequence that encodes a TCR δ variable domain, and in some cases, an unrearranged TCR Vα segment is able to rearrange with an unrearranged TCR Dδ segment and an unrearranged TCR Jδ segment to form a TCR Vα/DδJδ gene sequence that encodes a hybrid TCR α/δ variable domain. Upon rearrangement, a T cell matures and enters the periphery. As such, somatic cells that do not express a T cell maturation marker, e.g., CD3⁻ somatic cells, may also comprise a “germline” or unrearranged T cell receptor variable region sequence.

The TCRα locus (chromosome 14 for mouse and human) comprises a cluster of Vα gene segments, each preceded by an exon encoding the leader sequence (L). A cluster of Jα gene segments is located a considerable distance from the Vα gene segments. The Jα gene segments are followed by a single α constant (C) region gene sequence, which contains separate exons for the constant and hinge domains and a single exon encoding the transmembrane and cytoplasmic regions. The TCRβ locus (chromosome 6 for mouse, chromosome 7 for human) has a different organization, with a cluster of Vβ gene segments located distantly from two separate clusters, (e.g., a TCRBDJ1 cluster and a TCRBDJ2 cluster)—each containing a single D gene segment, together with six or seven J gene segments and a single Cβ gene. Each TCR Cβ gene has separate exons encoding the constant domain, the hinge, the transmembrane region, and the cytoplasmic region. The TCRα locus is interrupted between the V and J gene segments by another T-cell receptor locus—the TCRδ locus.

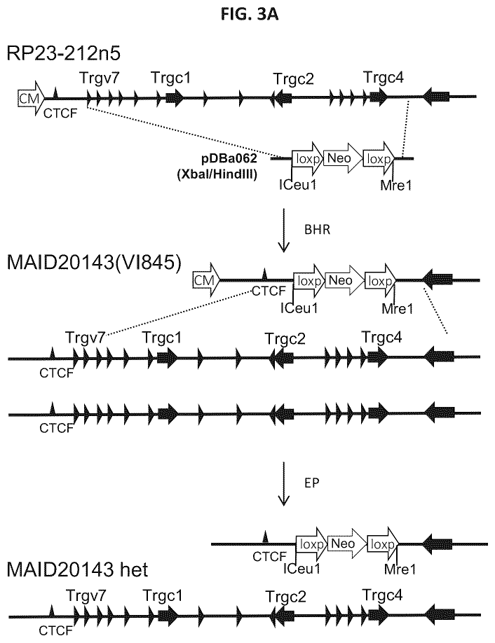

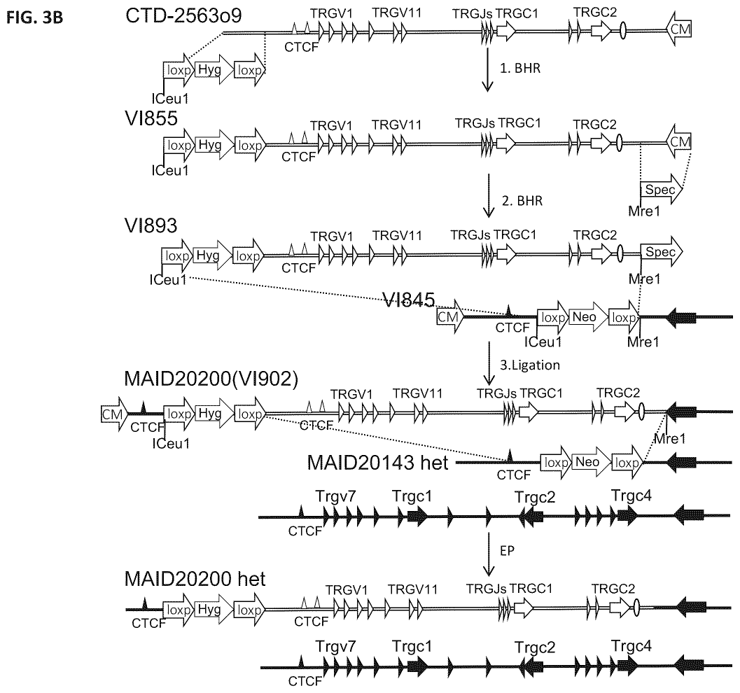

The genomic organization of the TCR γ locus and TCR δ locus are notably different than that of the TCR α locus or the TCR β locus. The TCR δ locus lies within the TCR α locus. The three Dδ gene segments, three Jδ gene segments, and the single δ C gene lie between the cluster of Vα gene segments and the cluster of Jα gene segments; the Vδ gene segments are interspersed among the Vα gene segments with a Vδ3 gene segment between the TCR Cδ gene and the cluster of Jα segments. See, Janeway's Immunobiology, Chapter 4, 5^{th} Ed., Murphy et al. eds., Garland Science, 2001. The TCR γ locus consists of four different constant (C) region gene sequences (3 functional) in mice and two γ constant region gene sequences (TCRGC1 and TCRGC2) in humans, with each Cγ region gene sequence containing its own cluster of Jγ gene segments. See, e.g., FIG. 3B.

A specific T cell receptor (TCR) repertoire is generated during T cell development through a complex developmental program in the thymus. Diversity of this repertoire is maintained by the expression of distinct TCR α/(3 or γ/δ chains and by their variable region encoded by several variable (V), joining (J), and diversity (D) gene segments.

Generally, it is understood that TCR gene segments rearrange during T cell development to form complete variable domain exons. TCRα variable (Vα) and joining (Jα) gene segments undergo rearrangement, such that the resultant TCR α chain is encoded by a specific combination of VJ segments (Vα/Jα sequence) operably linked to a TCR α constant (Cα) region gene sequence; the TCRβ variable (Vβ), diversity (Dβ), and joining (Jβ) gene segments undergo rearrangement such that the resultant TCR (3 chain is encoded by a specific combination of VDJ segments (Vβ/Dβ/Jβ sequence) operably linked to a TCR β constant (Cβ) region gene sequence; the TCRγ variable (Vγ) and joining (Jγ) gene segments undergo rearrangement such that the resultant TCR γ chain is encoded by a specific combination of VJ segments (Vγ/Jγ sequence) operably linked to a TCR γ (Cγ) gene; the TCRδ variable (Vδ), diversity (Dδ) and joining (Jδ) gene segments undergo rearrangement such that the resultant TCR δ chain is encoded by a specific combination of VDJ segments (Vδ/Dδ/Jδ sequence), and sometimes VDDJ (Vδ/Dδ/Dδ/Jδ sequence) operably linked to a TCR δ (Cδ) gene. See, Hata et al. (188) Science 240:1541-1544; Hata et al. (1989) J. Exp. Med. 169:41-57. The use of two D segments greatly increases the variability of the δ chain, mainly because extra N-region nucleotides can be added at the junction between the two D gene segments as well as at the V-D and D-J junctions.

TCR diversity may be mostly attributed to combinatorial and junctional diversity generated during the process of gene rearrangement. Most of the variability in TCR chains is found in the junctional region encoded by V, D, and J gene segments and modified by P- and N-nucleotides. This region encodes the CDR3 loops in TCR chains that form the center of the antigen-binding site. Thus, the center of the TCR chain will be highly variable, whereas the periphery will be subject to relatively little variation.

Interactions with thymic stroma trigger thymocytes to undergo several developmental stages, characterized by expression of various cell surface markers. A summary of characteristic cell surface markers at various developmental stages in the thymus is presented in Table 1. Rearrangement at the TCRβ variable gene locus begins at the DN2 stage and ends during the DN4 stage, while rearrangement of the TCRα variable gene locus occurs at the DP stage. After the completion of TCRβ locus rearrangement, the cells express TCRβ chain at the cell surface together with the surrogate a chain, pTα. See, Janeway's Immunobiology, Chapter 7, 7^{th} Ed., Murphy et al. eds., Garland Science, 2008.

TABLE 1
Developmental Stages of T cells in the Thymus

| | Developmental Stage | | | | | |
|---|---|---|---|---|---|---|
| | DN1 | DN2 | DN3 | DN4 | DP | SP |
| Marker(s) | CD44+/ | CD44+/ | CD44^{low}/ | CD44−/ | CD4+/ | CD4+ |
| | CD25− | CD25+ | CD25+ | CD25− | CD8+ | or |
| | | | | | | CD8+ |

Naive CD4+ and CD8+ T cells exit the thymus and enter the peripheral lymphoid organs (e.g., spleen) where they are exposed to antigens and are activated to clonally expand and differentiate into a number of effector T cells (Teff), e.g., cytotoxic T cells, T_{REG} cells, T_{H}17 cells, T_{H}1 cells, T_{H}2 cells, etc. Subsequent to infection, a number of T cells persist as memory T cells, and are classified as either central memory T cells (Tcm) or effector memory T cells (Tem). Sallusto et al. (1999) Two subsets of memory T lymphocytes with distinct homing potentials and effector functions, Nature 401:708-12 and Commentary by Mackay (1999) Dual personality of memory T cells, Nature 401:659-60. Sallusto and colleagues proposed that, after initial infection, Tem cells represent a readily available pool of antigen-primed memory T cells in the peripheral tissues with effector functions, while Tcm cells represent antigen-primed memory T cells in the peripheral lymphoid organs that upon secondary challenge can become new effector T cells. While all memory T cells express CD45RO isoform of CD45 (naïve T cells express CD45RA isoform), Tcm are characterized by expression of L-selectin (also known as CD62L) and CCR7+, which are important for binding to and signaling in the peripheral lymphoid organs and lymph nodes. Id. Thus, all T cells found in the peripheral lymphoid organs (e.g., naïve T cells, Tcm cells, etc.) express CD62L. In addition to CD45RO, all memory T cells are known to express a number of different cell surface markers, e.g., CD44. For summary of various cell surface markers on T cells, see Janeway's Immunobiology, Chapter 10, supra.

Early studies led scientists to believe that α/β and γ/δ T cells develop in a sequential manner, e.g., that T cells would develop from developing α/β thymocytes when γ/δ TCR rearrangements are nonfunctional. Allison, J., et al., Immunol Today (1987) 8:293-296; Pardoll, D., et al., Nature (1987) 326:79-81; Boismenu R Curr. Biol. (1995) 5:829-831; each reference is incorporated herein in its entirety by reference. However, studies from genetically modified mice expressing integrated rearranged TCR γ and/or TCR δ sequences (KN6 mice with a Vγ4Jγ1Cγ1 and a Vδ5DJδ1Cδ rearranged sequences) in their germlines contradicted the sequential rearrangement model since such expression did not prevent the normal development of α/β T cells. It was also shown that knockout of TCR Cα genes had no effect on the generation of γ/δ thymocytes or the development of γ/δ T cells, and instead, resulted in an increase in γ/δ thymocyte numbers that may derive from noncanonical development pathways. Knockout of TCR CP in mice also showed no developmental block in γ/δ T cell development, and it was thus concluded that double positive cells seem not to be intermediates in the development of TCR γ/δ T cells. Kreslavasky, T., et al. (2010) Curr Opin Immunol. 22:185-192; Mombaerts P., et al., (1992) 360:225-231. Other extensive studies with various knockout and TCR transgenic mice suggest several different models regarding the requirements for the lineage decision and maturation into specific T cell lineages, reviewed in Hahn, A. M., and Winkler, T. H., (2020) J. Leuk. Bio. 107:2019, incorporated herein in its entirety by reference. A stochastic model proposes that fate determination occurs stochastically before TCR expression. Using Vγ4-Jγ1Cγ1 modified mice, it was demonstrated that IL-7Rα high cells show biased differentiation towards the γ/δ fate. A signal strength model, based on studies with Vγ6Jγ1Cγ1 and Vδ1Dδ1Jδ2Cδ modified mice, proposes the strength of the TCR signal is a critical determinant for lineage fate determination and that γ/δ TCR signaling may be a fundamental parameter for the thymic differentiation of γ/δ T cell subsets. Hayes, S. M., et al. (2002) Immunity, 16:827-38; Hayes, S. M., et al. (2005) Immunity, 22:583-93; Haks et al. (2005) Immunity, 22:595-606; Jensen, K. D., et al. (2008) Immunity 29:90-100; Dent A., et al. (1990) Nature 406:524; Sim, G., et al. (1995) J. Immunol. 154:5827-31.

While TCR variable domain functions primarily in antigen recognition, the extracellular portion of the constant domain, as well as transmembrane, and cytoplasmic domains of the TCR also serve important functions. A complete TCR receptor complex requires more than the α and β, or γ and δ polypeptides; additional molecules required include CD3γ, CD3δ, and CD3ε, as well as the ζ chain homodimer (ζζ). At the completion of TCRβ rearrangement, when the cells express TCRβ/pTα, this pre-TCR complex exists together with CD3 on the cell surface. TCRα (or pTα) on the cell surface has two basic residues in its transmembrane domain, one of which recruits a CD3γε heterodimer, and another recruits CC via their respective acidic residues. TCRβ has an additional basic residue in its transmembrane domain that is believed to recruit CD3δε heterodimer. See, e.g., Kuhns et al. (2006) Deconstructing the Form and Function of the TCR/CD3 Complex, Immunity 24:133-39; Wucherpfennig et al. (2009) Structural Biology of the T-cell Receptor: Insights into Receptor Assembly, Ligand Recognition, and Initiation of Signaling, Cold Spring Harb. Perspect. Biol. 2:α005140. The assembled complex, comprising TCRα|3 heterodimer, CD3γε, CD3δε, and CC, is expressed on the T cell surface. The polar residues in the transmembrane domain have been suggested to serve as quality control for exiting endoplasmic reticulum; it has been demonstrated that in the absence of CD3 subunits, TCR chains are retained in the ER and targeted for degradation. See, e.g., Call and Wucherpfennig (2005) The T Cell Receptor: Critical Role of the Membrane Environment in Receptor Assembly and Function, Annu. Rev. Immunol. 23:101-25.

CD3 and ζ chains of the assembled complex provide components for TCR signaling as TCR αβ heterodimer (or TCR γ/δ heterodimer) by itself lacks signal transducing activity. The CD3 chains possess one Immune-Receptor-Tyrosine-based-Activation-Motif (ITAM) each, while the ζ chain contains three tandem ITAMs. ITAMs contain tyrosine residues capable of being phosphorylated by associated kinases. Thus, the assembled TCR-CD3 complex contains 10 ITAM motifs. See, e.g., Love and Hayes (2010) ITAM-Mediated Signaling by the T-Cell Antigen Receptor, Cold Spring Harb. Perspect. Biol. 2:e002485. Following TCR engagement, ITAM motifs are phosphorylated by Src family tyrosine kinases, Lck and Fyn, which initiates a signaling cascade, resulting in Ras activation, calcium mobilization, actin cytoskeleton rearrangements, and activation of transcription factors, all ultimately leading to T cell differentiation, proliferation, and effector actions. Id., see also, Janeway's Immunobiology, supra; both incorporated herein by reference.

Additionally, TCRβ transmembrane and cytoplasmic domains are thought to have a role in mitochondrial targeting and induction of apoptosis; in fact, naturally occurring N-terminally truncated TCRβ molecules exist in thymocytes. Shani et al. (2009) Incomplete T-cell receptor—β peptides target the mitochondrion and induce apoptosis, Blood 113:3530-41. Thus, several important functions are served by the TCR constant domain (which, in various embodiments, comprises a portion of extracellular as well as transmembrane and cytoplasmic domains); and in various embodiments the structure of this region should be taken into consideration when designing humanized TCRs or genetically modified non-human animals expressing the same.

The signal strength model relates to the observation that the γ/δ TCR has a different signaling potential CD3 complex composition. Hayes, S. M. and Love P. E., (2002) Immunity, supra. γ/δ T cell development also begins at the immature DN stage; however, γ/δ T lineage cells do not proceed through developmental stages defined by the expression of a pre-TCR or CD4/CD8 coreceptors. Instead, in-frame rearrangements of both the TCR γ and TCR δ genes in DN thymocytes result in the surface expression of the mature γ/δ TCR, which transduces signals thought to regulate the commitment of immature DN cells to the γ/δ T cell lineage and to be required for the differentiation into mature γ/δ T cells. The structure of the γ/δ TCR has been reported to resemble that of the α/β TCR, except that the γ/δ TCR can include the FcϵRI γ (FcRγ) chain. Qian, D., et al. (1993) Proc. Natl. Acad. Sci. USA 90:11875-879; Park, S. W., et al. (1995) Eur. J. Immunol. 25:2107-2110, each of which reference is incorporated in its entirety herein by reference. Deletion of CD3γ, CD3ϵ, or CD3 blocks γ/δ T cell development, whereas loss of CD3δ or FcRγ has no effect on the generation of mature γ/δ TCR⁺ cells. Hayes et al. (2002) Immunity show that despite the absence of CD3δ, signal transduction by the γ/δ TCR is superior to that of the α/β TCR, as measured by its ability to induce calcium mobilization, MAPKinase activation, and cellular proliferation.

Gamma/delta (γ/δ) T cells remain less characterized. T cells bearing γ/δ TCRs are a distinct lineage of T cells and appear to be able to recognize antigen directly, much as antibodies do, without the requirement for presentation by an MHC molecule or processing of the antigen, and also to recognize a broad range of antigens (e.g., phosphoproteins, lipids, glycolipids) independent of MHC. Although rare in peripheral blood, γ/δ T cells are detectable in the thymus, spleen, and lymph nodes, and highly abundant in select tissue sites such as gut tissues (e.g., colon, intestines (e.g., small intestines)) and skin under continual surveillance for microbial and other environmental antigens (J. C. Ribot, N. Lopes, B. Silva-Santos, γ/δ T cells in tissue physiology and surveillance. Nat Rev Immunol 21,221-232 (2021); incorporated herein in its entirety by reference). Relative to α/β T cells, γ/δ cells have a more restricted TCR repertoire and acquire specific cytokine production and tissue homing properties early in development. These features, along with the lack of MHC restriction and recognition of antigens associated with ‘danger’ signals, illustrate properties of this T lymphoid subset conventionally linked with ‘innate’ immunity (B. Silva-Santos, S. Mensurado, S. B. Coffelt, γ/δ T cells: pleiotropic immune effectors with therapeutic potential in cancer. Nature Reviews Cancer, 1-13 (2019); incorporated herein in its entirety by reference).

Described herein are genetically modified non-human animals (e.g., rodents, e.g., rats, mice) that may be used as an animal model of human or humanized cellular responses involving γ/δ T cells to study such responses, develop human therapeutics, etc. Generally, a genetically modified non-human animal as described herein comprises unrearranged human or humanized (e.g., human γ and/or δ; and/or human α and/or β) T cell variable gene loci that are capable of rearranging (or rearrange), e.g., in a T cell, to form nucleic acid sequences that encode human T cell receptor variable domains, including animals that comprise T cells that comprise rearranged human variable domains, and human or non-human (e.g., mouse or rat) constant domains. Also described herein are non-human animals (e.g., rodents, e.g., rats, mice) that are capable of generating a diverse repertoire of human T cell receptor variable region sequences; thus, the present invention provides non-human animals that express TCRs with fully human variable domains (e.g., human TCR γ variable domains and/or human TCR δ variable domains) and/or fully human TCRs (e.g., human TCR γ chains and/or human TCR δ chains) in response to an antigen of interest and that bind an epitope of the antigen of interest. In some embodiments, provided are non-human animals that generate a diverse T cell receptor repertoire (γ/δ T cells and α/β T cells) capable of reacting with various antigens, including but not limited to antigens not presented by APCs (recognized by γ/δ T cells) and antigens presented by APCs (recognized by α/β T cells).

In one embodiment, the invention provides genetically modified non-human animals (e.g., rodents, e.g., rats, mice) that comprise in their genome unrearranged human TCR variable region segments (V(D)J segments), wherein the unrearranged human TCR variable region segments replace, at an endogenous non-human (e.g., rodent) TCR variable gene locus (e.g., TCRγ and/or TCRδ loci, and/or TCRα and/or TCRβ loci), endogenous non-human TCR variable region segments. In one embodiment, unrearranged human TCR variable gene locus replaces endogenous non-human TCR variable gene locus.

In another embodiment, the invention provides genetically modified non-human animals (e.g., rodents, e.g., rats, mice) that comprise in their genome unrearranged human TCR variable region segments (V(D)J segments), wherein the unrearranged human TCR variable region segments are operably linked to a human or non-human TCR constant region gene sequence resulting in a human or humanized TCR locus, respectively, wherein the human or humanized TCR locus is at a site in the genome other than the endogenous non-human TCR locus. Thus, in one embodiment, a non-human animal (e.g., rodent, e.g., mouse, rat) comprising a transgene that comprises unrearranged human TCR variable region segments operably linked to human or non-human TCR constant region gene sequence is also provided.

In some embodiments, the genetically modified non-human animals of the invention comprise in their genome (a) human TCR variable region segments operably linked to (b) human or retained non-human (e.g., rodent, e.g., mouse, rat) TCR constant region gene sequence(s) that encode TCR constant domains. As indicated above, the constant domain of the TCR participates in a signaling cascade initiated during antigen-primed T cell activation; thus, a TCR constant domain interacts with a variety of anchor and signaling proteins in the T cell. Thus, in one aspect, the genetically modified non-human animals of the invention express human or humanized T cell receptors that retain the ability to recruit a variety of endogenous non-human anchor or signaling molecules, e.g., CD3 molecules (e.g., CD3γ, CD3δ, CD3ε), the ζ chain, Lck, Fyn, ZAP-70, etc. A nonlimiting list of molecules that are recruited to the TCR complex is described in Janeway's Immunobiology, supra. In some embodiments, a non-human animal as described therein, or a cell of the non-human animal, comprises a human T cell polypeptide (e.g., a human TCR γ polypeptide and/or a human δ polypeptide) as described herein associated with a non-human animal (e.g., rodent, e.g., rat or mouse) TCR anchor or signaling molecule, e.g., a CD3 molecules (e.g., CD3γ, CD3δ, CD3ε), the ζ chain, Lck, Fyn, ZAP-70, etc., and/or a non-human animal FcϵRI γ (FcRγ) chain.

Accordingly, in various embodiments, the invention generally provides genetically modified non-human animals wherein the non-human animals comprise in the genome unrearranged humanized TCR variable gene loci, e.g., an unrearranged human TCR variable gene region comprising human TCR variable segments capable of recombining (or recombine), e.g., in a T cell (e.g., of a mouse), to form a human rearranged TCR variable gene sequence. TCR locus or TCR gene locus, as used herein, refers to the position in a genomic DNA comprising the TCR coding region, including the entire TCR coding region, including unrearranged V(D)J sequences, enhancer sequence, constant region gene sequence(s), and any upstream or downstream (UTR, regulatory regions, etc.), or intervening DNA sequence (introns, etc.). TCR variable locus, TCR variable region, or TCR variable gene locus refers to the position in a genomic DNA that includes TCR variable region segments (V(D)J region) but excludes TCR constant region gene sequences and, in various embodiments, enhancer sequences. Other sequences may be included in the TCR variable gene locus for the purposes of genetic manipulation (e.g., selection cassettes, restriction sites, etc.), and these are encompassed herein. In various aspects, the non-human animals comprise contiguous portions of the human genomic TCR variable loci that comprise V, D, and J, or D and J, or V and J, or V segments arranged as in an unrearranged human genomic variable locus, e.g., comprising promoter sequences, leader sequences, intergenic sequences, regulatory sequences, etc., arranged as in a human genomic TCR variable locus. A contiguous human sequence in reference to TCRG, TCRD, TCRA and/or TCRB sequence(s) may also generally refer to a fully human sequence, e.g., wherein both the TCR coding sequences (e.g., TCR gene segments) and TCR non-coding sequences (e.g., non-coding DNA that separates and is flanked by TCR gene segments, such as non-coding recombination signal sequences (RSSs) and other non-coding intergenic sequences) are human, and preferably, wherein the TCR gene segments and TCR non-coding sequences are in the same sequential order in which they can be found in the human germline.

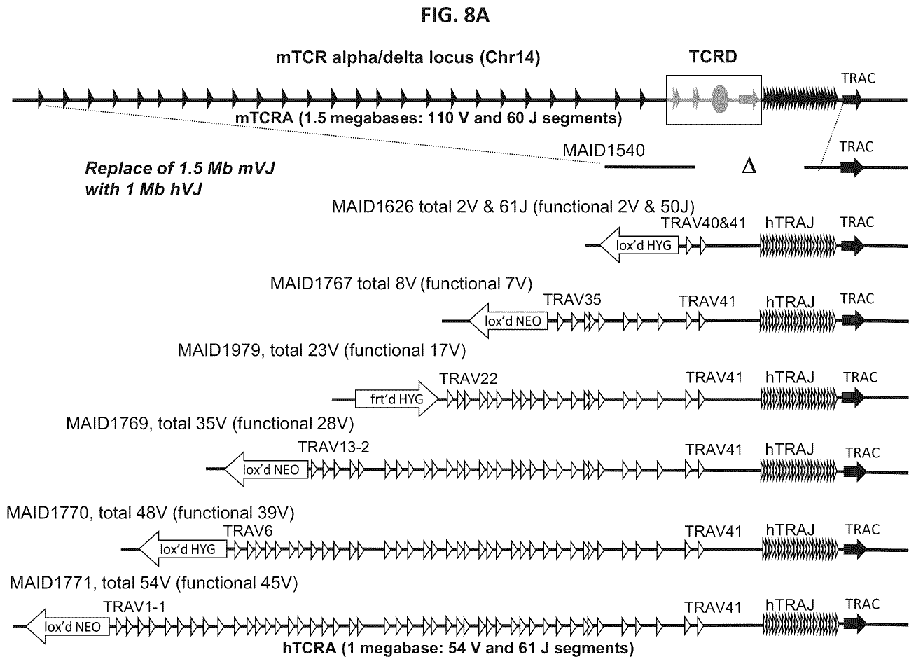

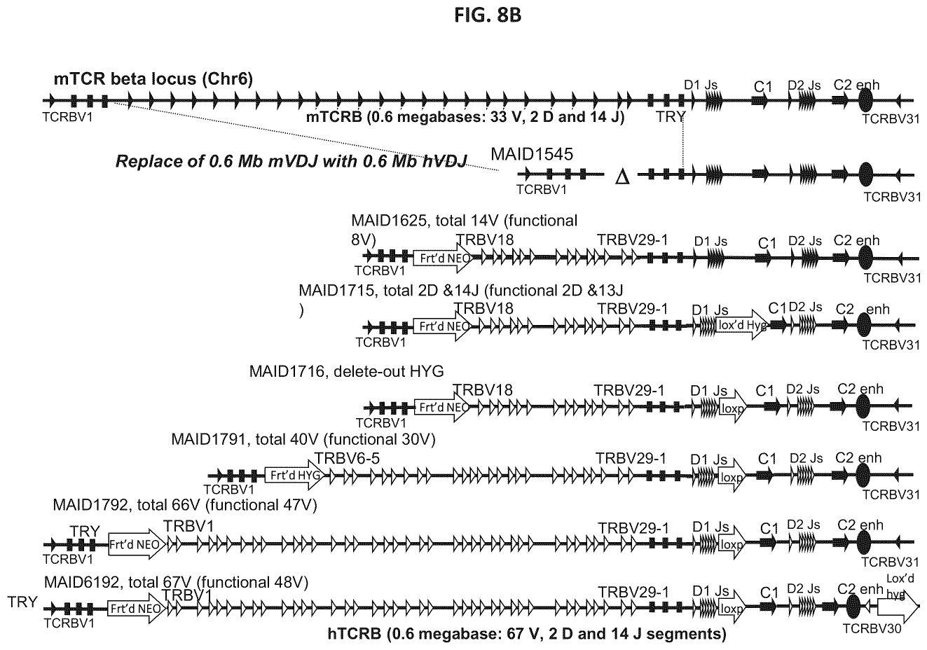

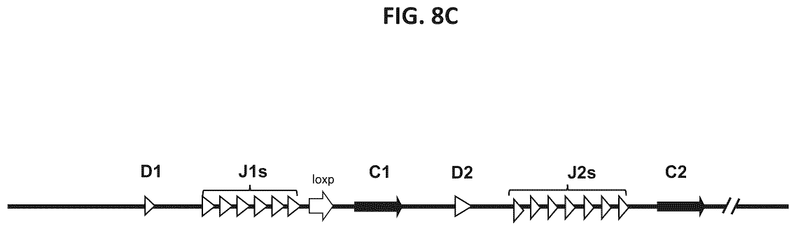

In various embodiments of the human or humanized TCR γ locus and/or human and/or humanized TCR δ locus and optionally human or humanized TCR α and/or TCR β loci, the humanized locus can comprise human coding sequences (e.g., TCR gene segments) and non-human, e.g., murine, TCR non-coding sequences. In some embodiments, the human TCR gene segments replace orthologous non-human (e.g., mouse) TCR gene segments such that the human TCR gene segments flank the same non-human (e.g., mouse) TCR non-coding sequences as those that are flanked by the replaced orthologous non-human (e.g., mouse) TCR gene segments, e.g., such that the human TCR gene segments and non-human (e.g., mouse) TCR non-coding sequences are in the same order as that would be found in the non-human (e.g., mouse) germline but for the replacements of the orthologous (e.g., non-human) TCR gene segments. See, e.g., FIG. 8C.

In other aspects, the various segments are arranged as in an unrearranged non-human genomic TCR variable locus. In various embodiments of the humanized TCR α, β, δ and/or γ locus, the humanized locus can comprise two or more human genomic segments that do not appear in a human genome juxtaposed, e.g., a fragment of V segments of the human variable locus located in a human genome proximal to the constant region, juxtaposed with a fragment of V segments of the human variable locus located in a human genome at the upstream end of the human variable locus.

Human or Humanized γ and/or δ TCR Responses

In some embodiments, a non-human animal is provided that comprises in its genome unrearranged human TCRγ variable region segments, wherein the unrearranged human TCRγ variable region segments are operably linked to a human or non-human TCRγ constant region gene sequence resulting in a human or humanized TCRγ locus, respectively. In one embodiment, the human or humanized TCRγ locus is at a site in the genome other than the endogenous non-human TCRγ locus. In another embodiment, the unrearranged human TCRγ variable region segments replace endogenous non-human TCRγ variable region segments and human TCR γ constant region gene sequence(s) replace(s) endogenous non-human TCRγ constant region gene sequence(s), (e.g., wherein a nucleotide sequence comprising an endogenous TCRγ constant region gene sequence (e.g., an endogenous Trgc1 constant region gene sequence, an endogenous Trgc2 constant region gene sequence, an endogenous Trgc3 constant region gene sequence, and/or an endogenous Trgc4 constant region gene sequence) is replaced with a nucleotide sequence comprising a human TCRγ constant region gene sequence, (e.g., a human TRGC1 constant region sequence and/or a human TRGC2 constant region sequence). In one embodiment, the unrearranged human TCRγ variable gene locus replaces endogenous non-human TCRγ variable gene locus.

In some embodiments, the unrearranged TCRγ variable gene locus comprising human variable region segments (e.g., human Vγ and Jγ segments) is positioned in the non-human genome such that the human variable region segments replace corresponding non-human variable region segments. In one embodiment, the unrearranged TCRγ variable gene locus comprising human variable region segments replaces endogenous TCRγ variable gene locus. In one aspect, endogenous non-human Vγ and Jγ segments are incapable of rearranging to form a rearranged Vγ/Jγ sequence. Thus, in one aspect, the human Vγ and Jγ segments in the unrearranged TCRγ variable gene locus are capable of rearranging (or rearrange), e.g., in a T cell (e.g., of a mouse), to form a rearranged human Vγ/Jγ sequence.

In some embodiments, a non-human animal of the invention comprises an unrearranged humanized TCRγ locus, e.g., a TCRγ locus comprising at least one functional unrearranged human Vγ segment and at least one functional unrearranged human Jγ segment (e.g., a complete repertoire of functional unrearranged human Vγ segments and a complete repertoire of functional unrearranged human Jγ variable region segment). In some embodiments, a non-human animal of the invention comprises an unrearranged human TCRγ locus, e.g., a TCRγ locus comprising at least one functional unrearranged human Vγ segment and at least one functional unrearranged human Jγ segment (e.g., a complete repertoire of functional unrearranged human Vγ segments and a complete repertoire of functional unrearranged human Jγ segments, operably linked to at least one functional human TCR Cγ gene (e.g., a complete repertoire of functional human TCR Cγ genes), optionally wherein the functional unrearranged human Vγ segment(s), functional unrearranged human Jγ segment(s) and functional human TCR Cγ gene are in the same order as is found in a germline human TCRγ locus. The number and locations of various TCRγ segments can be determined from the IMGT database. The human TCRγ locus is on human chromosome 7, while the mouse TCRγ locus is on mouse chromosome 13.

The mouse TCRγ variable locus is approximately 200 kilobases and comprises 7 TRGV gene segments belonging to 5 subgroups, 4TRGJ gene segments, and 4 TRGC genes (3 functional). The human TCRγ variable locus is approximately 160 kilobases and comprises 12-15 TRGV (TRGV1, TRGV2, TRGV3, TRGV3P, TRGV4, TRGV5, TRGV5P, TRGV6, TRGV7, TRGV8, TRGV9, TRGV10, TRGV11, TRGVA, TRGVB) belonging to 6 subgroups, upstream of a duplicated J-C cluster, which comprises for the first part, 3 TRGJ (TRGJP1, TRGJP, and TRGJ1) and the TRGC1 gene, and for the second part, 2 TRGJ (TRGJP2 and TRGJ2) and the TRGC2 gene. Although not depicted in FIG. 3B, MAID20200 comprises all 15 hTRGV segments, all 5 hTRGJ segments, and both hTRGC genes in germline configuration. Unless stated otherwise, the numbers of human V(D)J segments referred to throughout the specification refers to the total number of V(D)J segments. In one embodiment of the invention, the genetically modified non-human animal (e.g., rodent, e.g., mouse or rat) comprises at least one human Vγ and at least one human Jγ segment. In some embodiments, the non-human animal comprises a human or humanized TCRγ locus that comprises 1, 2, 3, 4, 5, 6, 7, 8, 9, 10, 11, 12, 13, 14, 15, or more human Vγ segments. In some embodiments, the human or humanized TCRγ locus comprises at least one of the functional human Vγ gene segments. In some embodiments, the human or humanized TCRγ locus comprises two of the functional human Vγ gene segments. In some embodiments, the human or humanized TCRγ locus comprises three of the functional human Vγ gene segments. In some embodiments, the human or humanized TCRγ locus comprises four of the functional human Vγ gene segments. In some embodiments, the human or humanized TCRγ locus comprises five of the functional human Vγ gene segments. In some embodiments, the human or humanized TCRγ locus comprises six of the functional human Vγ gene segments. In some embodiments, the functional human Vγ gene segments are selected from the group consisting of human TRGV2, human TRGV3, human TRGV4, human TRGV5, human TRGV8, human TRGV9, and variants thereof. Thus, in some embodiments, the humanized TCRγ locus in the non-human animal may comprise 5%, 10%, 15%, 20%, 25%, 30%, 35%, 40%, 45%, 50%, 55%, 60%, 65%, 70%, 75%, 80%, 85%, 90%, 95%, 96%, 97%, 98% 99%, or 100% of human Vγ; in some embodiments, it may comprise about 2%, about 3%, about 15%, about 65%, about 90%, or 100% of human Vγ. In some embodiments, the non-human animal comprises a human or humanized TCRγ locus that comprises 1, 2, 3, 4, 5, 6, 7, 8, 9, 10, 11, 12, 13, 14, 15, or more human Vγ segments. In some embodiments, the non-human animal comprises a human or humanized TCRγ locus that comprises a human TRGJP1 gene segment. In some embodiments, the non-human animal comprises a human or humanized TCRγ locus that comprises a human TRGJP gene segment. In some embodiments, the non-human animal comprises a human or humanized TCRγ locus that comprises a human TRGJ1 gene segment. In some embodiments, the non-human animal comprises a human or humanized TCRγ locus that comprises a human TRGJP2 gene segment. In some embodiments, the non-human animal comprises a human or humanized TCRγ locus that comprises a human TRGJ2 gene segment. In some embodiments, the non-human animal comprises an endogenous TCRγ locus that comprises, in operable linkage to the human TRGV and TRJV segments, a human TRGC1 gene. In some embodiments, the non-human animal comprises an endogenous TCRγ locus that comprises, in operable linkage to the human TRGV and TRJV segments, a human TRGC2 gene.

In one embodiment, the non-human animal comprises a human TCRγ locus that comprises a DNA fragment comprising a contiguous human sequence of human Vγ1 (Vγ segment is also referred to as “TRGV” or “TCRGV”)) to human Cγ2 (“TCRGC2”; Cγ is also referred to as “TRGC” or “TCRGC”) which includes, between the human Vγ gene segments and human Cγ2 gene, human Jγ gene segment segments (Jγ segment is also referred to as “TRGJ” or “TCRGJ”) and human Cγ1 (“TCGRC1”) gene. A TCRG non-coding sequence refers to a contiguous non-coding sequence comprising non-coding recombinant signal sequences (RSSs) and other non-coding intergenic sequences found between any two consecutive unrearranged TRGV segments, between any unrearranged TRGV segment and unrearranged TRGJ segment, and between any two consecutive unrearranged TRGJ segments.

In some embodiments, a humanized TCR γ mouse as described herein comprises:
(I) germ cells and CD3− somatic cells that comprise an unrearranged TCR γ variable region sequence comprising an unrearranged human TCR Vγ segment and an unrearranged human TCR Jγ segment,
   wherein the unrearranged TCR γ variable region sequence is operably linked to a human TCRγ constant region gene sequence, optionally at an endogenous TCRγ locus, (e.g., a human TCRγ constant region gene sequence at an endogenous TCRγ locus, wherein a nucleotide sequence comprising an endogenous TCRγ constant region gene sequence (e.g., an endogenous Trgc1 constant region gene sequence, an endogenous Trgc2 constant region gene sequence, an endogenous Trgc3 constant region gene sequence, and/or an endogenous Trgc4 constant region gene sequence) is replaced with a nucleotide sequence comprising a human TCRγ constant region gene sequence, (e.g., a human TRGC1 constant region sequence and/or a human TRGC2 constant region sequence),
   wherein the unrearranged human TCR Vγ segment and the unrearranged human TCR Jγ segment are capable of rearranging (or rearrange) in a T cell to form a rearranged human TCR Vγ/Jγ variable region gene that is operably linked to the human TCR γ constant region gene sequence, and
   wherein the rearranged human TCR Vγ/Jγ variable region gene operably linked to the human TCR γ constant region gene sequence together encode a human TCR γ polypeptide, and
(II) a CD3⁺ T cell that expresses on its surface a functional TCR that comprises a human TCR γ polypeptide.

In some embodiments, the human TCR γ polypeptide is derived from a human TRGV2 gene segment, a human TRGV3 gene segment, a human TRGV4 gene segment, a human TRGV5 gene segment, a human TRGV8 gene segment, a human TRGV9 gene segment, a human TRGV10 gene segment, or a human TRGV11 gene segment. In some embodiments, the human TCR γ polypeptide is derived from a human TRGJ1 gene segment, a human TRGJP gene segment, a human TRGJP1 gene segment, a human TCRGJ2 gene segment, or a human TRGJP2 gene segment.

In some humanized TCR γ mouse embodiments, the germ cells and CD3⁻ somatic cells further comprise an unrearranged T cell receptor (TCR) δ variable region sequence comprising an unrearranged human TCR Vδ segment, an unrearranged human TCR Dδ segment, and an unrearranged human TCR Jδ segment, wherein the unrearranged TCR δ variable region sequence is operably linked to a human TCR δ constant region gene sequence, optionally at an endogenous TCR δ locus, and wherein the unrearranged human TCR Vδ segment, the unrearranged human TCR Dδ segment, and the unrearranged human TCR Jδ segment are capable of rearranging (or rearrange) in a T cell to form a rearranged human TCR Vδ/Dδ/Jδ variable region gene that is operably linked to the human TCR δ constant region gene sequence, and wherein the rearranged human TCR Vδ/Dδ/Jδ variable region gene operably linked to the human TCR δ constant region gene sequence together encode a human TCR δ polypeptide, wherein the mouse further comprises a CD3⁺ T cell that expresses, on its surface, a functional TCR that comprises both a human TCR γ polypeptide and a human TCR δ polypeptide.

In some embodiments, the human TCR δ polypeptide is derived from a human TRDV1 gene segment, a human TRAV17 gene segment, a human TRAV19 gene segment, a human TRAV21 gene segment, a human TRAV21 gene segment, a human TRAV26-2 gene segment, a human TRAV29/TRDV5 gene segment, a human TRAV31 gene segment, a human TRAV38-2/DV8 gene segment, a human TRAV39 gene segment, a human TRAV40 gene segment, a human TRAV41 gene segment, a human TRDV2 gene segment, or a human TRDV3 gene segment. In some embodiments, the human TCR δ polypeptide is derived from a human TRDJ1 gene segment, a human TRDJ2 gene segment, a human TRDJ3 gene segment, or a human TRDJ4 gene segment.

In some embodiments, a non-human animal comprises (with or without a human or humanized TCR γ locus as described herein) in its genome unrearranged human TCRδ variable region segments, wherein the unrearranged human TCRδ variable region segments are operably linked to a human or non-human TCRδ constant region gene sequence resulting in a human or humanized TCRδ locus, respectively. In one embodiment, the humanized TCRδ locus is at a site in the genome other than the endogenous non-human TCRδ locus. In another embodiment, the unrearranged human TCRδ variable region segments replace endogenous non-human TCRδ variable region segments and human TCR δ constant region gene sequence(s) replace(s) endogenous non-human TCRδ constant region gene sequence(s). In one embodiment, the unrearranged human TCRδ variable gene locus replaces endogenous non-human TCRδ variable gene locus.

In some embodiments, the unrearranged TCRδ variable gene locus comprising human variable region segments (e.g., human Vδ, Dδ, and Jδ segments) is positioned in the non-human genome such that the human variable region segments replace corresponding non-human variable region segments. In one embodiment, the unrearranged TCRδ variable gene locus comprising human variable region segments replaces endogenous TCRδ variable gene locus. In one aspect, endogenous non-human Vδ, Dδ, and Jδ segments are incapable of rearranging to form a rearranged Vδ/Dδ/Jδ sequence. Thus, in one aspect, the human Vδ, Dδ, and Jδ segments in the unrearranged TCRδ variable gene locus are capable of rearranging (or rearrange), e.g., in a T cell, to form a rearranged human Vδ/Dδ/Jδ sequence.

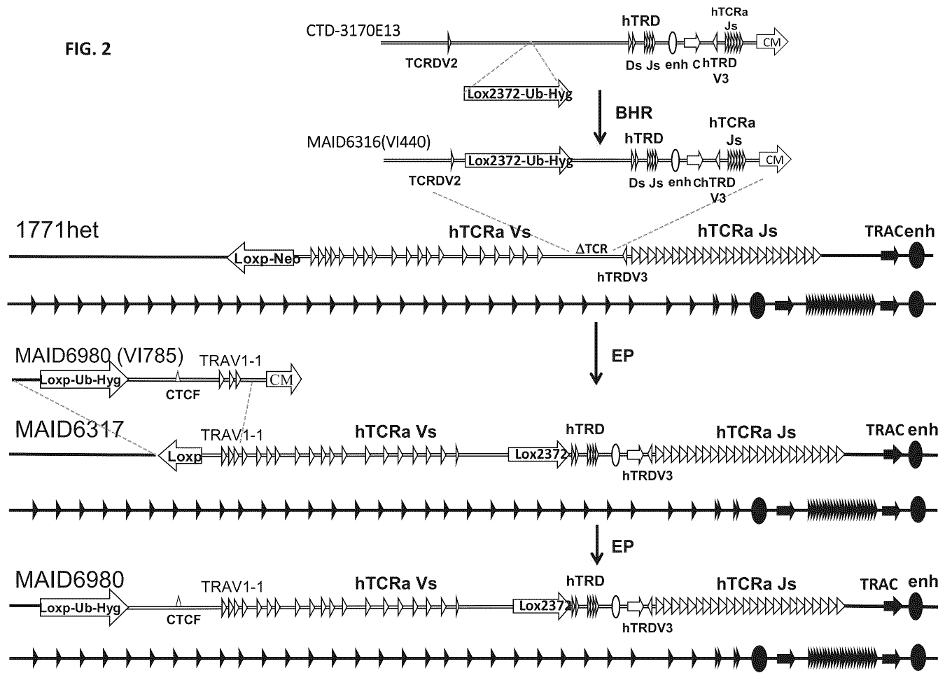

In some embodiments, a non-human animal of the invention comprises an unrearranged humanized TCRδ locus, e.g., a TCRδ locus comprising at least one functional unrearranged human Vδ segment, at least one functional unrearranged human Dδ segment, and at least one functional unrearranged human Jδ segment (e.g., a complete repertoire of functional unrearranged human Vδ segments, a complete repertoire of functional unrearranged human Dδ segments, and a complete repertoire of functional unrearranged human Jδ segments). In some embodiments, a non-human animal of the invention comprises an unrearranged human TCR locus, e.g., a TCRδ locus comprising at least one functional unrearranged human Vδ segment, at least one functional unrearranged human Dδ segment, and at least one functional unrearranged human Jδ segment (e.g., a complete repertoire of functional unrearranged human Vδ segments, a complete repertoire of functional unrearranged human Dδ segments, and a complete repertoire of functional unrearranged human Jδ segments), operably linked to a functional human TCR Cδ gene, optionally wherein the functional unrearranged human Vδ segment(s), functional unrearranged human Dδ segment(s), functional unrearranged human Jδ segment(s) and functional human TCR Cδ gene are in the same order as is found in a germline human TCR locus. The number and locations of various TCRδ segments can be determined from the IMGT database. In both mouse and human, the TCRδ gene segments are located within the TCRα locus on chromosome 14 (see FIG. 2). TCRδ J and D segments are located between Vα and Jα segments, while TCRδ V segments are interspersed throughout the TCRα locus, with the majority located among various Vα segments. Due to the genomic arrangement of TCRδ gene segments within the TCRα locus, successful rearrangement at the TCRα locus may delete or inactivate the TCRδ gene segments.

The mouse TCRδ variable locus is approximately 275 kilobases and comprises 6 TRDV (16 including 10 upstream TRAV/DV), 2 TRDD, 2TRDJ, and 1 TRDC. One TRDV is in inverted orientation 3′ of the TRDC. The human TCR cluster localized between the TRAV and TRAJ gene segments comprises 1 TRDV gene segment (TRDV2), 3 TRDD gene segments (TRDD1, TRDD2, TRDD3), 4 TRDJ gene segments (TRDJ1, TRDJ4, TRDJ2, and TRDJ3), 1 TRDC gene, and downstream of the TRDC gene, 1 TRDV in inverted orientation (TRDV3). This cluster spans 60 kilobases. One TRDV gene segment (TRDV1) is localized among the TRAV gene segments, and 5 gene segments described as TRAV/DV gene segments (TRAV14/DV4, TRAV23/DV6, TRAV29/DV5, TRAV36/DV7, and TRAV38-2/DV8) within the TRAV gene segments can be used for the synthesis of TCR δ chains or TCR α chains. In one embodiment, the genetically modified non-human animal (e.g., rodent, e.g., mouse or rat) comprises at least one human Vδ, one human Dδ, and at least one human Jδ gene segment. In one embodiment, the non-human animal comprises a human TCRδ locus that comprises 1 human Vδ segment. In one embodiment, the non-human animal comprises a human TCRδ locus that comprises 2 human Vδ segments. In one embodiment, the non-human animal comprises a human TCRδ locus that comprises 3 human Vδ segments. In one embodiment, the non-human animal comprises a human TCRδ locus that comprises 1 human TRAV/DV segment. In one embodiment, the non-human animal comprises a human TCRδ locus that comprises 2 human TRAV/DV segments. In one embodiment, the non-human animal comprises a human TCR locus that comprises 3 human TRAV/DV segments. In one embodiment, the non-human animal comprises a human TCRδ locus that comprises 4 human TRAV/DV segments. In one embodiment, the non-human animal comprises a human TCRδ locus that comprises 5 human TRAV/DV segments. In some embodiments, the non-human animal comprises a human TCR locus that comprises 1 human TRDJ gene segment. In some embodiments, the non-human animal comprises a human TCRδ locus that comprises 2 human TRDJ gene segments. In some embodiments, the non-human animal comprises a human TCRδ locus that comprises 3 human TRDJ gene segments. In some embodiments, the non-human animal comprises a human TCR locus that comprises 4 human TRDJ gene segments. In some embodiments, the non-human animal comprises a human TCRδ locus that comprises a human TRDC gene.

In one embodiment, the non-human animal comprises a humanized TCRδ locus that comprises a DNA fragment comprising a contiguous human sequence comprising human Vδ1 to Vδ3 gene segments (Vδ segment is also referred to as “TRDV” or “TCRDV”), which includes Dδ1, Dδ2, Dδ3 (Dδ segment is also referred to as “TRDD” or “TCRDD”), and a Cδ gene (Cδ is also referred to as “TRDC” or “TCRDC”). In some embodiments, the DNA fragment also comprises human Jα segments. A TCRD non-coding sequence refers to a contiguous non-coding sequence comprising non-coding recombinant signal sequences (RSSs) and other non-coding intergenic sequences found between any two consecutive unrearranged TRDV segments, between any unrearranged TRDV segment and unrearranged TRDD segment, between any two consecutive unrearranged TRDD segments, and between any TRDD segment and TRCD gene. In various embodiments, the DNA fragments comprising contiguous human sequences of human TCRδ variable region segments also comprise restriction enzyme sites, selection cassettes, endonucleases sites, or other sites inserted to facilitate cloning and selection during the locus humanization process. In various embodiments, these additional sites do not interfere with proper functioning (e.g., rearrangement, splicing, etc.) of various genes at the TCRδ locus.

In some embodiments, a mouse as described herein comprises germ cells and CD3⁻ somatic cells that comprise:
(A) a replacement of an endogenous TCR Vγ segment with an unrearranged human TCR Vγ segment, a replacement of an endogenous TCR Jγ segment with an unrearranged human TCR Jγ segment, and a replacement of an endogenous TCR γ constant region gene sequence with a human TCR γ constant region gene sequence; or
(B) a replacement of an endogenous TCR Vδ segment with an unrearranged human TCR Vδ segment, a replacement of an endogenous TCR Dδ segment with an unrearranged human TCR Dδ segment, a replacement of an endogenous TCR Jδ segment with an unrearranged human TCR Jδ segment, and a replacement of an endogenous TCR δ constant region gene sequence with a human TCR δ constant region gene sequence; or
(C) (i) a replacement of an endogenous TCR Vγ segment with an unrearranged human TCR Vγ segment, a replacement of an endogenous TCR Jγ segment with an unrearranged human TCR Jγ segment, and a replacement of an endogenous TCR γ constant region gene sequence with human TCR γ constant region gene sequence, and
   (ii) a replacement of an endogenous TCR Vδ segment with an unrearranged human TCR Vδ segment, a replacement of an endogenous TCR Dδ segment with an unrearranged human TCR Dδ segment, a replacement of an endogenous TCR Jδ segment with an unrearranged human TCR Jδ segment, and a replacement of an endogenous TCR δ constant region gene sequence with a human TCR δ constant region gene sequence.

In some embodiments,
(A) the unrearranged TCR Vγ segment comprises a full repertoire of unrearranged human TCR Vγ segments and the unrearranged human TCR Jγ segment comprises a full repertoire of unrearranged human TCR Jγ segments; or
(B) the unrearranged human TCR Vδ segment comprises a full repertoire of unrearranged human TCR Vδ segments, the unrearranged human TCR Dδ segment comprises a full repertoire of unrearranged human TCR Dδ segments, and the unrearranged human TCR Jδ segment comprises a full repertoire of unrearranged human TCR Jδ segments; or
(C) (i) the unrearranged TCR Vγ segment comprises a full repertoire of unrearranged human TCR Vγ segments and the unrearranged human TCR Jγ segment comprises a full repertoire of unrearranged human TCR Jγ segments, and
   (ii) the unrearranged human TCR Vδ segment comprises a full repertoire of unrearranged human TCR Vδ segments, the unrearranged human TCR Dδ segment comprises a full repertoire of unrearranged human TCR Dδ segments, and the unrearranged human TCR Jδ segment comprises a full repertoire of unrearranged human TCR Jδ segments.

In some embodiments, a mouse as described herein comprises:
(I) germ cells and CD3⁻ T cells that comprise:
   (A) at the endogenous TCR γ locus:
      a replacement of all endogenous TCR Vγ segments with a full repertoire of unrearranged human TCR Vγ segments,
      a replacement of all endogenous TCR Jγ segments with a full repertoire of unrearranged human TCR Jγ segments, and
      a replacement of all TCR γ constant region gene sequences with a full repertoire of human TCR γ constant region gene sequences; and
   (B) at the endogenous TCR δ locus:
      a replacement all endogenous TCR Vδ segments with a full repertoire of unrearranged human TCR Vδ segments,
      a replacement of all endogenous TCR Dδ segments with a full repertoire of unrearranged human TCR Dδ segments,
      a replacement of all endogenous TCR Dδ segments with a full repertoire of unrearranged human TCR Jδ segments, and
      a replacement of an endogenous TCR δ constant region gene sequence with a human TCR δ constant region gene sequence; and
   (II) a CD3⁺ T cell that expresses, on its surface, a functional TCR comprising a human TCR γ polypeptide and a human TCR δ polypeptide.

In some embodiments, a non-human animal as described herein with humanized TRD and/or TRG loci have γ/δ T cells in the spleen and thymus, which make up a small fraction of the total T cells as is observed in WT mice (FIGS. 5A-5B), as well as in the skin and gut mucosa (FIGS. 12A-12D). In addition, TCR repertoire analysis of thymus and spleen mRNA revealed successful diverse V(D)J gene usage, indicating proper recombination, expression and thymic selection of humanized γ/δ TCRs (FIGS. 6A, 6B, 7A, and 7B). Recombination of a diverse repertoire of V(D)J gene segments was also seen in γ/δ T cells in skin and gut mucosa (data not shown).

Since the TRD locus is located within the TRA locus, in some embodiments, germ cells and CD3⁻ somatic cells as described herein further comprise an unrearranged human TCR Vα segment upstream of the unrearranged TCR δ variable region sequence and the human TCR δ constant region gene sequence, wherein the unrearranged human TCR Vα segment, the unrearranged human TCR Dδ, and the unrearranged human TCR Jδ segment are capable of rearranging (or rearrange) in a T cell to form a rearranged human TCR Vα/Dδ/Jδ variable region gene that is operably linked to the human TCR δ constant region gene sequence, wherein the rearranged human TCR Vα/Dδ/Jδ variable region gene sequence operably linked to the human TCR δ constant region gene sequence together encode a human hybrid TCR polypeptide comprising a human hybrid TCR α/δ variable domain and a human TCR δ constant domain, and wherein the mouse further comprises a CD3⁺ T cell that expresses, on its surface, the human hybrid TCR α/δ variable domain operably linked to the human TCR δ constant domain.

In some embodiments, a humanized TCR γ and/or humanized TCR δ non-human animal as described herein may also have a human or humanized TCR α locus. Mice with a human or humanized α locus are described in U.S. Pat. No. 9,113,616, incorporated herein by reference. See also FIG. 8A.

### Fully Humanized T Cell Responses

Described herein are non-human animals comprising, along with human or humanized TRD and TRG loci, human or humanized TRA and/or TRB loci and human or humanized loci of other components involved with α/β TCR rearrangement and/or activation, e.g., loci of TCR co-receptors (CD4 and CD8) and MHC.

Human or Humanized α and/or β TCR Loci

In some embodiments, a non-human animal is provided that comprises in its genome unrearranged human TCRα variable region segments, wherein the unrearranged human TCRα variable region segments are operably linked to a human or non-human TCRα constant region gene sequence resulting in a human or humanized TCRα locus, respectively. In one embodiment, the human or humanized TCRα locus is at a site in the genome other than the endogenous non-human TCRα locus. In another embodiment, the unrearranged human TCRα variable region segments replace endogenous non-human TCRα variable region segments and human TCR α constant region gene sequence(s) replace(s) endogenous non-human TCRα constant region gene sequence(s). In one embodiment, the unrearranged human TCRα variable gene locus replaces endogenous non-human TCRα variable gene locus.

In one embodiment, the unrearranged TCRα variable gene locus comprising human variable region segments (e.g., human Vα and Jα segments) is positioned in the non-human genome such that the human variable region segments replace corresponding non-human variable region segments. In one embodiment, the unrearranged TCRα variable gene locus comprising human variable region segments replaces endogenous TCRα variable gene locus. In one aspect, endogenous non-human Vα and Jα segments are incapable of rearranging to form a rearranged Vα/Jα sequence. Thus, in one aspect, the human Vα and Jα segments in the unrearranged TCRα variable gene locus are capable of rearranging (or rearrange), e.g., in a T cell, to form a rearranged human Vα/Jα sequence.

In some embodiments, a non-human animal of the invention comprises an unrearranged humanized TCRα locus, e.g., a TCRα locus comprising at least one functional unrearranged human Vα segment and at least one functional unrearranged human Jα segment (e.g., a complete repertoire of functional unrearranged human Vα segments and a complete repertoire of functional unrearranged human Jα variable region segment). In some embodiments, a non-human animal of the invention comprises an unrearranged human TCRα locus, e.g., a TCRα locus comprising at least one functional unrearranged human Vα segment and at least one functional unrearranged human Jα segment (e.g., a complete repertoire of functional unrearranged human Vα segments and a complete repertoire of functional unrearranged human Jα segments, operably linked to a functional TCR Cα gene (e.g., an endogenous TCR Cα gene), optionally wherein the functional unrearranged human Vα segment(s), functional unrearranged human Jα segment(s) and functional TCR Cα gene are in the same order as is found in a germline human TCRα locus. The number and locations of various TCRα segments can be determined from the IMGT database.

The mouse TCRα variable locus is approximately 1.5 megabases and comprises a total of 110Vα and 60 Jα segments. The human TCRα variable locus is approximately 1 megabase and comprises a total of 54Vα and 61Jα segments, with 45Vα and 50Jα believed to be functional. Unless stated otherwise, the numbers of human V(D)J segments referred to throughout the specification refers to the total number of V(D)J segments. In one embodiment of the invention, the genetically modified non-human animal (e.g., rodent, e.g., mouse or rat) comprises at least one human Vα and at least one human Jα segment. In one embodiment, the non-human animal comprises a humanized TCRα locus that comprises 1, 2, 3, 4, 5, 6, 7, 8, 9, 10, 15, 20, 23, 25, 30, 35, 40, 45, 48, 50, or up to 54 human Vα segments. In some embodiments, the humanized TCRα locus comprises 2, 8, 23, 35, 48, or 54 human Vα segments. Thus, in some embodiments, the humanized TCRα locus in the non-human animal may comprise 5%, 10%, 15%, 20%, 25%, 30%, 35%, 40%, 45%, 50%, 55%, 60%, 65%, 70%, 75%, 80%, 85%, 90%, 95%, 96%, 97%, 98% 99%, or 100% of human Vα; in some embodiments, it may comprise about 2%, about 3%, about 15%, about 65%, about 90%, or 100% of human Vα.

In one embodiment, the non-human animal comprises a humanized TCRα locus that comprises a DNA fragment comprising a contiguous human sequence of human Vα40 to Vα41 (Vα segment is also referred to as “TRAV” or “TCRAV”) and a DNA fragment comprising a contiguous human sequence of 61 human Jα segments (Jα segment is also referred to as “TRAJ” or “TCRAJ”). A TCRA non-coding sequence refers to a contiguous non-coding sequence comprising non-coding recombinant signal sequences (RSSs) and other non-coding intergenic sequences found between any two consecutive unrearranged TRAV segments, between any unrearranged TRAV segment and unrearranged TRAJ segment, and between any two consecutive unrearranged TRAJ segments. In one embodiment, the non-human animal comprises a humanized TCRα locus that comprises a DNA fragment comprising a contiguous human sequence of human TRAV35 to TRAV41 and a DNA fragment comprising a contiguous human sequence of 61 human TRAJs. In one embodiment, the non-human animal comprises a humanized TCRα locus that comprises a DNA fragment comprising a contiguous human sequence of human TRAV22 to TRAV41 and a DNA fragment comprising a contiguous human sequence of 61 human TRAJs. In one embodiment, the non-human animal comprises a humanized TCRα locus that comprises a DNA fragment comprising a contiguous human sequence of human TRAV13-2 to TRAV41 and a DNA fragment comprising a contiguous human sequence of 61 human TRAJs. In one embodiment, the non-human animal comprises a humanized TCRα locus that comprises a DNA fragment comprising a contiguous human sequence of human TRAV6 to TRAV41 and 61 human TRAJs. In one embodiment, the non-human animal comprises a humanized TCRα locus that comprises a DNA fragment comprising a contiguous human sequence of human TRAV1-1 to TRAV 41 and 61 human TRAJs. In various embodiments, the DNA fragments comprising contiguous human sequences of human TCRα variable region segments also comprise restriction enzyme sites, selection cassettes, endonucleases sites, or other sites inserted to facilitate cloning and selection during the locus humanization process. In various embodiments, these additional sites do not interfere with proper functioning (e.g., rearrangement, splicing, etc.) of various genes at the TCRα locus.

In one embodiment, the humanized TCRα locus comprises 61 human Jα segments, or 100% of human Jα segments. In a particular embodiment, humanized TCRα locus comprises 8 human Vα segments and 61 human Jα segments; in another particular embodiment, humanized TCRα locus comprises 23 human Vα segments and 61 human Jα segments. In another particular embodiment, the humanized TCRα locus comprises a complete repertoire of human Vα and Jα segments, i.e., all human variable α region gene segments encoded by the α locus, or 54 human Vα and 61 human Jα segments. In various embodiments, the non-human animal does not comprise any endogenous non-human Vα or Jα segments at the TCRα locus.

In some embodiments, a mouse as described herein comprises:
(I) germ cells and CD3⁻ somatic cells that comprise from 5′ to 3:
   an unrearranged human TCR Vα segment and
   an unrearranged TCR δ variable region sequence comprising an unrearranged human TCR Vδ segment, an unrearranged TCR Dδ, and an unrearranged human TCR Jδ segment,
      wherein the unrearranged TCR δ variable region sequence is operably linked to a human TCR δ constant region gene sequence, optionally at an endogenous TCR δ locus,
      wherein the unrearranged human TCR Vα segment, the unrearranged human TCR Dδ, and the unrearranged human TCR Jδ segment are capable of rearranging (or rearrange) in a T cell to form a rearranged human TCR Vα/Dδ/Jδ variable region gene that is operably linked to the human TCR δ constant region gene sequence,
      wherein the rearranged human TCR Vα/Dδ/Jδ variable region gene sequence operably linked to the human TCR δ constant region gene sequence together encode a human hybrid TCR polypeptide comprising a human hybrid TCR α/δ variable domain and a human TCR δ constant domain, and
(II) a CD3⁺ T cell that expresses, on its surface, a functional TCR that comprises the human hybrid TCR α/δ variable domain operably linked to the human TCR δ constant domain.

In some embodiments, the germ cells and CD3⁻ somatic cells of a mouse described herein may comprise a replacement an endogenous TCR Vα segment with the unrearranged human TCR Vα segment and a replacement of an endogenous TCR Jα segment with an unrearranged human TCR Jα segment, wherein the unrearranged human TCR Vα segment and unrearranged human TCR Jα segment are operably linked to each other and a TCR α constant region gene sequence, such as a mouse TCR α constant region gene sequence, and wherein the unrearranged human TCR Vα segment and unrearranged human TCR Jα segment are capable of rearranging (or rearrange) in a T cell to form a rearranged TCR Vα/Jα variable region gene operably linked to the TCR α constant region gene sequence, wherein the rearranged human TCR Vα/Jα variable region gene operably linked to the TCR α constant region gene sequence together encode a TCR α polypeptide comprising a human TCR α variable domain, and wherein the mouse further comprises a CD3⁺ T cell that expresses, on its surface, a functional TCR comprising the TCR α polypeptide. In some embodiments, the germ cells and CD3⁻ somatic cells comprise a replacement of all endogenous TCR Vα segments with a full repertoire of unrearranged human TCR Vα segments and a replacement of all endogenous TCR Jα segments with a full repertoire of unrearranged human TCR Jα segments, wherein the full repertoire of unrearranged human TCR Vα segments and full repertoire of unrearranged human TCR Jα segments are operably linked to each other and a TCR α constant region gene sequence at an endogenous TCR α locus, wherein the full repertoire of unrearranged human TCR Vα segments and the full repertoire of unrearranged human TCR Jα segments are capable of rearranging (or rearrange) in a T cell to form a rearranged human TCR Vα/Jα variable region gene operably linked to the endogenous TCR α constant region gene sequence, wherein the rearranged TCR Vα/Jα variable region gene operably linked to the endogenous TCR α constant region gene sequence together encode a chimeric TCR α polypeptide comprising a human TCR α variable domain operably linked to a mouse TCR α constant domain, and wherein the mouse further comprises a CD3⁺ T cell that expresses, on its surface, a functional TCR that comprises the chimeric TCR α polypeptide.

The humanized TRA/D and TRG loci may be introduced into mice with humanized TRB loci and other components of T cell immunity, including loci of TCR co-receptors (CD4 and CD8) and MHC. These mice will therefore bear full humanization of both T cell lineages.

As a non-limiting example, the humanized TRD and/or TRG loci may be introduced into mice comprising human or humanized TRB loci (e.g., TRB loci comprising humanized TCRBDJ1 and TCRBDJ2 clusters with murine TCRB noncoding sequences and human coding sequences), such as those mice described in U.S. Pat. No. 9,113,616 and. Moore, M. et al. Sci Immunol 6 (2021); doi:10.1126/sciimmunol.abj4026; each of which is incorporated herein in its entirety by reference. See FIG. 8B and FIG. 8C.

In some embodiments, a non-human animal is provided that comprises in its genome unrearranged human TCRβ variable region segments, wherein the unrearranged human TCRβ variable region segments are operably linked to a non-human TCRβ constant region gene sequence resulting in a humanized TCRβ locus. In one embodiment, the humanized TCRβ locus is at a site in the genome other than the endogenous non-human TCRβ locus. In another embodiment, the unrearranged human TCRβ variable region segments replace endogenous non-human TCRβ variable region segments while retaining endogenous non-human TCRβ constant region gene sequence(s). In one embodiment, the unrearranged human TCRβ variable gene locus replaces endogenous non-human TCRβ variable gene locus.

In some embodiments, the unrearranged TCRβ variable gene locus comprising human variable region segments (e.g., human Vβ, Dβ, and JP segments) is positioned in the non-human genome such that the human variable region segments replace corresponding non-human variable region segments. In one embodiment, the unrearranged TCRβ variable gene locus comprising human variable region segments replaces endogenous TCRβ variable gene locus. In one aspect, endogenous non-human Vβ, Dβ, and Jβ segments are incapable of rearranging to form a rearranged Vβ/Dβ/Jβ sequence. Thus, in one aspect, the human Vβ, Dβ, and Jβ segments in the unrearranged TCRβ variable gene locus are capable of rearranging (or rearrange), e.g., in a T cell, to form a rearranged human Vβ/Dβ/Jβ sequence.

The mouse TCRβ variable locus is approximately 0.6 megabases and comprises a total of 33 Vβ, 2 Dβ, and 14 Jβ segments. The human TCRβ variable locus is approximately 0.6 megabases and comprises a total of 67 Vβ, 2 Dβ, and 14 Jβ segments. In one embodiment of the invention, the genetically modified non-human animal (e.g., rodent, e.g., mouse or rat) comprises at least one human Vβ, at least one human Dβ, and at least one human Jα segment.

In one embodiment, the non-human animal comprises a humanized TCRβ locus that comprises 1, 2, 3, 4, 5, 6, 7, 8, 9, 10, 15, 20, 23, 25, 30, 35, 40, 45, 48, 50, 55, 60, or up to human 67 Vβ segments. In some embodiments, the humanized TCRβ locus comprises 8, 14, 40, 66, or human 67 Vβ segments. Thus, in some embodiments, the humanized TCRβ locus in the non-human animal may comprise 5%, 10%, 15%, 20%, 25%, 30%, 35%, 40%, 45%, 50%, 55%, 60%, 65%, 70%, 75%, 80%, 85%, 90%, 95%, 96%, 97%, 98% 99%, or 100% of human Vβ; in some embodiments, it may comprise about 20%, about 60%, about 15%, about 98%, or 100% of human Vβ.

In some embodiments, an endogenous TCRβ variable gene locus, e.g., an endogenous TCRβ mouse variable gene locus, comprises:
a replacement of one or all of the contiguous endogenous T cell variable region Vβ gene segments, e.g., one or all contiguous endogenous T cell variable region Vβ gene segments between a first 5′ trypsinogen cluster and a second 3′ trypsinogen cluster, with one or all unrearranged human T cell variable region gene segments from TRBV1 to TRBV29-1, and/or
a replacement of one or more non-contiguous endogenous Vβ gene segments (e.g., an endogenous mouse TCRBV31 gene segment) with a human TCRBV gene segment (e.g., a replacement of a mouse TCRBV31 gene segment with an orthologous human TCRBV30 gene segment).

In one embodiment, the non-human animal comprises a humanized TCRβ locus that comprises a DNA fragment comprising a contiguous human sequence of human Vβ18 to Vβ29-1 (Vβ segment is also referred to as “TRBV” or “TCRBV”). In one embodiment, the non-human animal comprises a humanized TCRβ locus that comprises a DNA fragment comprising a contiguous human sequence of human TRBV18 to TRBV29-1, a separate DNA fragment comprising a contiguous human TCRBDJ1 sequence comprising human D1314131 (i.e., human Dβ1-Jβ1-1-Jβ1-6 segments), and a separate DNA fragment comprising a contiguous human TCRBDJ2 sequence that comprises human Dβ2-Jβ2 (i.e., human Dβ2-Jβ2-1-Jβ2-7 segments). An unrearranged TCRBDJ1 sequence, which may also be referred to an unrearranged TCRBJD1 cluster, comprises an unrearranged TCRBD1 segment, one to all unrearranged TCRBJ1 segments (e.g., Jβ1-1, Jβ1-2, Jβ1-3, Jβ1-4, Jβ1-5, and Jβ1-6 segments), and TCRBDJ1 non-coding sequences between the unrearranged TCRBD1 segment and the unrearranged TCRBJ1 segment and between any two consecutive unrearranged TCRBJ1 gene segments. A TCRB non-coding sequence refers to a contiguous non-coding sequence comprising non-coding recombinant signal sequences (RSSs) and other non-coding intergenic sequences found between any two consecutive unrearranged TRBV segments, and may include a TCRBDJ1 non-coding sequence, e.g., a contiguous non-coding sequence comprising non-coding recombinant signal sequences (RSSs) and other non-coding intergenic sequences found between a unrearranged TRBD1 segment and a TRBJ1 segment and between any two consecutive unrearranged TRBJ1 segments, or a TCRBDJ2 non-coding sequence, e.g., a contiguous non-coding sequence comprising non-coding recombinant signal sequences (RSSs) and other non-coding intergenic sequences found between a unrearranged TRBD2 segment and a TRBJ2 segment and between any two consecutive unrearranged TRBJ2 segments. An unrearranged TCRBDJ1 sequence may be operably linked to a plurality of unrearranged TRBV segments and a TCRBC1 constant region gene sequence (which may also be referred to as a TRBC1 region sequence). An unrearranged TCRBDJ2 sequence, which may also be referred to an unrearranged TCRBJD2 cluster, comprises an unrearranged TCRBD2 segment, one to all unrearranged TCRBJ2 segments (e.g., Jβ2-1, Jβ2-2, Jβ2-3, Jβ2-4, Jβ2-5, Jβ2-6, and J(32-7 segments), and TCRBDJ2 non-coding sequences between the unrearranged TCRBD2 segment and the unrearranged TCRBJ2 segment and between any two consecutive unrearranged TCRBJ2 gene segments. An unrearranged TCRBDJ2 sequence may be operably linked to a plurality of unrearranged TRBV segments and a TCRBC2 constant region gene sequence (which may also be referred to as a TRBC2 region sequence). In one embodiment, the non-human animal comprises a humanized TCRβ locus that comprises either or both (i) a TCRBDJ1 cluster wherein at least all of or at least one of Dβ1-Jβ1 segments (i.e., Dβ1, Jβ1-1, Jβ1-2, Jβ1-3, Jβ1-4, Jβ1-5, and Jβ1-6 segments) are human and wherein the non-coding sequences between the Dβ1-Jβ1 segments, including RSSs and other intergenic sequences, are non-human, e.g., mouse, optionally wherein the Dβ1 and Jβ1-1 to JβJ1-6 segments flank the same mouse TCR non-coding sequences as are normally flanked by mouse Trbd1 and mouse Trbj1-1 to Trbj1-6 segments and/or (ii) a TCRBDJ2 cluster wherein at least one of or all of the Dβ2-Jβ2 segments (i.e., Dβ2, Jβ2-1, Jβ2-2, Jβ2-3, Jβ2-3, Jβ2-4, Jβ2-5, Jβ2-6, and Jβ2-7 segments) are human and wherein the non-coding sequences between the Dβ2-Jβ2 segments, including RSSs and other intergenic sequences, are non-human, e.g., mouse, optionally wherein the Dβ2 and JβJ2-1 to Jβ2-7 gene segments flank the same mouse TCR non-coding sequences as are normally flanked by the mouse Trbd2 and mouse Trbj2-1 to Trbj2-7 gene segments. In one embodiment, the non-human animal comprises a humanized TCRβ locus that comprises a DNA fragment comprising a contiguous human sequence of human TRBV6-5 to TRBV29-1, a separate DNA fragment comprising a contiguous human sequence of human Dβ1-Jβ1 (i.e., human Dβ1-Jβ1-1-Jβ1-6 segments), and a separate DNA fragment comprising a contiguous human sequence of human Dβ2-Jβ2 (i.e., human Dβ2-Jβ2-14β2-7 segments). In one embodiment, the non-human animal comprises a humanized TCRβ locus that comprises a DNA fragment comprising a contiguous human sequence of human TRBV1 to TRBV29-1, a separate DNA fragment comprising a contiguous human sequence of human Dβ1-Jβ1, and a separate DNA fragment comprising a contiguous human sequence of human Dβ2-Jβ2. In one embodiment, the non-human animal comprises a humanized TCRβ locus that comprises a DNA fragment comprising a contiguous human sequence of human TRBV1 to TRBV29-1, a separate DNA fragment comprising a contiguous human sequence of human Dβ1-Jβ1, a separate DNA fragment comprising a contiguous human sequence of human Dβ2-Jβ2, and a separate DNA fragment comprising the sequence of human TRBV30. In various embodiments, the DNA fragments comprising contiguous human sequences of human TCRβ variable region segments also comprise restriction enzyme sites, selection cassettes, endonucleases sites, or other sites inserted to facilitate cloning and selection during the locus humanization process. In various embodiments, these additional sites do not interfere with proper functioning (e.g., rearrangement, splicing, etc.) of various genes at the TCRβ locus.

In one embodiment, the humanized TCRβ locus comprises 14 human Jβ segments, or 100% of human Jβ segments, and 2 human DP segments or 100% of human DP segments. In another embodiment, the humanized TCRβ locus comprises at least one human Vβ segment, e.g., 14 human Vβ segments, and all mouse DP and Jβ segments. In a particular embodiment, humanized TCRβ locus comprises 14 human Vβ segments, 2 human DP segments, and 14 human Jβ segments. In another particular embodiment, the humanized TCRβ locus comprises a complete repertoire of human Vβ, Dβ, and Jβ segments, i.e., all human variable 13 region gene segments encoded by the β locus or 67 human Vβ, 2 human Dβ, and 14 human Jβ segments. In one embodiment, the non-human animal comprises one (e.g., 5′) non-human Vβ segment at the humanized TCRβ locus. In various embodiments, the non-human animal does not comprise any endogenous non-human Vβ, Dβ, or Jβ segments at the TCRβ locus.

In one embodiment, the humanized TCRβ locus comprises 13 human Jβ segments, or 100% of functional human Jβ segments, and 2 human DP segments or 100% of functional human Jβ segments. In another embodiment, the humanized TCRβ locus comprises at least one human Vβ segment, e.g., 14 human Vβ segments, and all functional mouse Dβ and Jβ segments. In a particular embodiment, humanized TCRβ locus comprises 14 human Vβ segments, 2 human Dβ segments, and 13 functional human Jβ segments. In another particular embodiment, the humanized TCRβ locus comprises a complete repertoire of human Vβ, Dβ, and Jβ segments, i.e., all human variable β region gene segments encoded by the β locus or 67 human Vβ, 2 human Dβ, and 13 functional human Jβ segments. In one embodiment, the non-human animal comprises one (e.g., 5′) non-human Vβ segment at the humanized TCRβ locus. In various embodiments, the non-human animal does not comprise any endogenous non-human Vβ, Dβ, or Jβ segments at the TCRβ locus.

In one aspect, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., CD4+ and/or CD8+ T cells, of which at least 10% of the TCR expressed by the population of spleen cells is derived from gene segments from the TCRBDJ1 cluster and at least 10% of the TCR expressed by the population of spleen cells is derived from gene segments from the TCRBDJ2 cluster. In some embodiments, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., CD4+ and/or CD8+ T cells, of which at least 15% of the TCR expressed by the population of spleen cells is derived from gene segments from the TCRBDJ1 cluster and at least 15% of the TCR expressed by the population of spleen cells is derived from gene segments from the TCRBDJ2 cluster. In some embodiments, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., CD4+ and/or CD8+ T cells, of which at least 20% of the TCR expressed by the population of spleen cells is derived from gene segments from the TCRBDJ1 cluster and at least 20% of the TCR expressed by the population of spleen cells is derived from gene segments from the TCRBDJ2 cluster. In some embodiments, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., CD4+ and/or CD8+ T cells, of which at least 30% of the TCR expressed by the population of spleen cells is derived from gene segments from the TCRBDJ1 cluster and at least 30% of the TCR expressed by the population of spleen cells is derived from gene segments from the TCRBDJ2 cluster. In some embodiments, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., CD4+ and/or CD8+ T cells, of which at least 40% of the TCR expressed by the population of spleen cells is derived from gene segments from the TCRBDJ2 cluster. In some embodiments, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., CD4+ and/or CD8+ T cells, of which at least 50% of the TCR expressed by the population of spleen cells is derived from gene segments from the TCRBDJ2 cluster. In some embodiments, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., CD4+ and/or CD8+ T cells, of which at least 60% of the TCR expressed by the population of spleen cells is derived from gene segments from the TCRBDJ2 cluster. In some embodiments, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., CD4+ and/or CD8+ T cells, of which at least 70% of the TCR expressed by the population of spleen cells is derived from gene segments from the TCRBDJ2 cluster. In some embodiments, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., a population of CD4+ and/or CD8+ T cells, which expresses TCRs derived from gene segments from the TCRBDJ1 cluster and TCRs derived from gene segments from the TCRBDJ2 cluster at a ratio of 1:3, 3:7, 1:2, 2:3, or 1:1. In some embodiments, the non-human animal (e.g., rodent, e.g., mouse or rat) comprising a humanized TCRβ variable gene locus that comprises non-human animal (e.g., rodent, e.g., mouse or rat) TCRB non-coding sequences as described herein comprises a population of spleen cells, e.g., a population of CD4+ and/or CD8+ T cells, which expresses TCRs derived from gene segments from the TCRBDJ2 cluster and TCRs derived from gene segments from the TCRBDJ1 cluster at a ratio of 1:3, 3:7, 1:2, or 2:3.

In some embodiments, the germ cells and CD3⁻ somatic cells of a mouse as described herein comprises an unrearranged TCRβ variable region sequence comprising at least one unrearranged human TCR variable region Vβ segment, at least one unrearranged human TCR variable region Dβ segment, and at least one unrearranged TCR variable region Jβ segment, wherein the unrearranged TCR β variable region sequence is operably linked to a TCR β constant region gene sequence, such as a mouse TCR β constant region gene sequence, optionally at an endogenous TCR β locus, and wherein the unrearranged human TCR Vβ segment, the unrearranged human TCR Dβ segment, and the unrearranged human TCR Jβ segment are capable of rearranging (or rearrange) in a T cell to form a rearranged human TCR Vβ/Dβ/Jβ variable region gene that is operably linked to the TCR β constant region gene sequence, and wherein the rearranged human TCR Vβ/Dβ/Jβ variable region gene operably linked to the TCR β constant region gene sequence together encode a TCR β polypeptide comprising a human TCR β variable domain; and wherein the mouse further comprises a CD3⁺ T cell that expresses on its surface a TCR comprising the TCR β polypeptide. In some embodiments, the unrearranged TCRβ variable region sequence comprises a mouse TCRB non-coding sequence.

In some mouse embodiments described herein,
(I) the germ cells and CD3⁻ somatic cells comprise:
   (A) at the endogenous TCR γ locus:
      a replacement of all endogenous TCR Vγ segments with a full repertoire of unrearranged human TCR Vγ segments,
      a replacement of all endogenous TCR Jγ segments with a full repertoire of unrearranged human TCR Jγ segments, and
      a replacement of all TCR γ constant region gene sequences with a full repertoire of human TCR γ constant region gene sequences; and
   (B) at the endogenous TCR δ locus:
      a replacement of all endogenous TCR Vδ segments with a full repertoire of unrearranged human TCR Vδ segments,
      a replacement of all endogenous TCR Dδ segments with a full repertoire of unrearranged human TCR Dδ segments,
      a replacement of all endogenous TCR Jδ segments with a full repertoire of unrearranged human TCR Jδ segments, and
      a replacement of an endogenous TCR δ constant region gene sequences with a human TCR δ constant region gene sequence; and
   (C) at the endogenous TCR α locus:
      a replacement of all endogenous TCR Vα segments with a full repertoire of unrearranged human TCR Vα segments, and
      a replacement of all endogenous TCR Jα segments with a full repertoire of unrearranged human TCR Jα segments, and
   (D) at the endogenous TCR β locus:
      a replacement of all endogenous TCR Vβ segments with a full repertoire of unrearranged human TCR Vβ segments,
      a replacement of all endogenous TCR Dβ segments with a full repertoire of unrearranged human TCR Dβ segments, and
      a replacement of all endogenous TCR Dβ segments with a full repertoire of unrearranged human TCR Jδ segments; and
(II) the mouse further comprises a CD3⁺ T cell that expresses, on its surface, a functional TCR comprising a human TCR γ polypeptide and a human TCR δ polypeptide and a CD3⁺ T cell that expresses, on its surface, a functional TCR comprising a human or humanized α polypeptide and a human or humanized TCR polypeptide.

In some embodiments, the germ cells and CD3⁻ somatic cells each comprises a human CTCF binding element. In some embodiments, the germ cells and somatic cells each comprises a human CTCF binding element upstream of a TCR α locus. In some embodiments, the germ cells and somatic cells each comprises a human CTCF binding element upstream of a TCR γ locus.

In some embodiments, the germ cells and somatic cells each comprises a human CTCF binding element upstream of a TCR α locus and a human CTCF binding element upstream of a TCR γ locus.

In one aspect, the non-human animal expresses a humanized T cell receptor with a non-human constant domain on the surface of a T cell, wherein the receptor is capable of interacting with non-human molecules, e.g., anchor or signaling molecules expressed in the T cell (e.g., CD3 molecules, the δ chain, or other proteins anchored to the TCR through the CD3 molecules or the δ chain). Thus, in one aspect, a cellular complex is provided, comprising (a) a non-human T-cell that expresses (i) a TCR that comprises a humanized TCRα chain as described herein and humanized TCRβ chain as described herein and (ii) a chimeric co-receptor as described herein and (b) a non-human antigen-presenting cell comprising an antigen bound to a chimeric MHC I and/or chimeric MHC II as described herein. In one embodiment, human TCRγ and/or TCRδ chains are complexed with a non-human, e.g., endogenous, zeta (C) chain homodimer and non-human, e.g., endogenous, CD3 heterodimers. In one embodiment, the cellular complex is an in vivo cellular complex.

In various embodiments, the non-human animals (e.g., rodents, e.g., mice or rats) described herein produce T cells that are capable of undergoing thymic development, progressing from DN1 to DN2 to DN3 to DN4 to DP and to CD4 or CD8 SP T cells. Such T cells of the non-human animal of the invention express cell surface molecules typically produced by a T cell during a particular stage of thymic development (e.g., CD25, CD44, Kit, CD3, pTα, etc.).

In various embodiments, the non-human animals described herein produce T cells that are capable of undergoing T cell differentiation in the periphery. In additional embodiments, the non-human animals described herein comprise CD3+ T cells in the periphery, e.g., in the spleen, skin, and gut mucosa.

DN1 and DN2 cells that do not receive sufficient signals (e.g., Notch signals) may develop into B cells, myeloid cells (e.g., dendritic cells), mast cells and NK cells. See, e.g., Yashiro-Ohtani et al. (2010) Notch regulation of early thymocyte development, Seminars in Immunology 22:261-69. In some embodiments, the non-human animals described herein develop B cells, myeloid cells (e.g., dendritic cells), mast cells and NK cells. In some embodiments, the non-human animals described herein develop a dendritic cell population in the thymus.

The predominant type of T cell receptors expressed on the surface of T cells is TCRα/β, with the minority of the cells expressing TCRδ/γ. In some embodiments, the T cells of the non-human animals comprising humanized TCRγ and/or δ loci exhibit utilization of TCRα/β and TCRγ/δ loci that is similar to the wild-type animal (e.g., the T cells of the non-human animals described herein express TCRα/β and TCRδ/γ proteins in comparable proportions to that expressed by wild type animals). Thus, in some embodiments, the non-human animals comprising optionally humanized TCRα/β and human TCR γδ loci exhibit utilization of all loci.

### Humanized T Cell Co-Receptor(s)

Although recognition of antigen by γ/δ T cells may not require interactions between T cell co-receptors (e.g., CD4 and CD8) and MHC, since a non-human animal as described herein may comprise, in addition to human or humanized TRD (and TRG) loci, human or humanized TRA and TRB loci, a non-human animal as described herein may also comprise a human or humanized CD4 locus and/or a human or humanized CD8 (e.g., CD8α and CD8β) locus, see, e.g., in U.S. Pat. Nos. 9,848,587 and 10,820,581, each of which is incorporated herein by reference.

Thus, disclosed herein are non-human animals that express at least one human or humanized T cell co-receptor, e.g., CD4, CD8α and/or CD8β. Accordingly, a non-human animal as disclosed herein comprises at least one of a first, second, and/or third nucleotide sequence, each of which encodes a different human or chimeric human/non-human T cell co-receptor polypeptide selected from a human or humanized CD4 polypeptide, a human or humanized CD8α polypeptide, and a human or humanized CD8β polypeptide. Use of the first, second, third designations herein is not to be construed as limiting the non-human animals disclosed herein as requiring all three nucleotide sequences or the presence of any of the co-receptor nucleotide sequences in any order. Accordingly, a non-human animal as disclosed herein may comprise a nucleic acid sequence or nucleic acid sequences encoding a human or humanized CD4 and/or a human or humanized CD8 (e.g., human or humanized CD8α and/or CD8β) polypeptide(s).

In one embodiment, a non-human animal as disclosed herein comprises a first nucleotide sequence encoding a human or humanized CD4 polypeptide. In another embodiment, a non-human animal as disclosed herein comprises a first nucleotide sequence encoding a human or humanized CD8α polypeptide and a second nucleotide sequence encoding a human or humanized CD8β polypeptide. In another embodiment, a non-human animal as disclosed herein comprises first and second nucleotide sequences encoding human or humanized CD8α and CD8β polypeptides and further comprises a third nucleotide sequence encoding a human or humanized CD4 polypeptide.

In various embodiments, the invention generally provides genetically modified non-human animals that comprise in their genome, e.g., at an endogenous CD4 locus, a nucleotide sequence encoding a human or humanized CD4 polypeptide; thus, the animals express a human or humanized CD4 polypeptide.

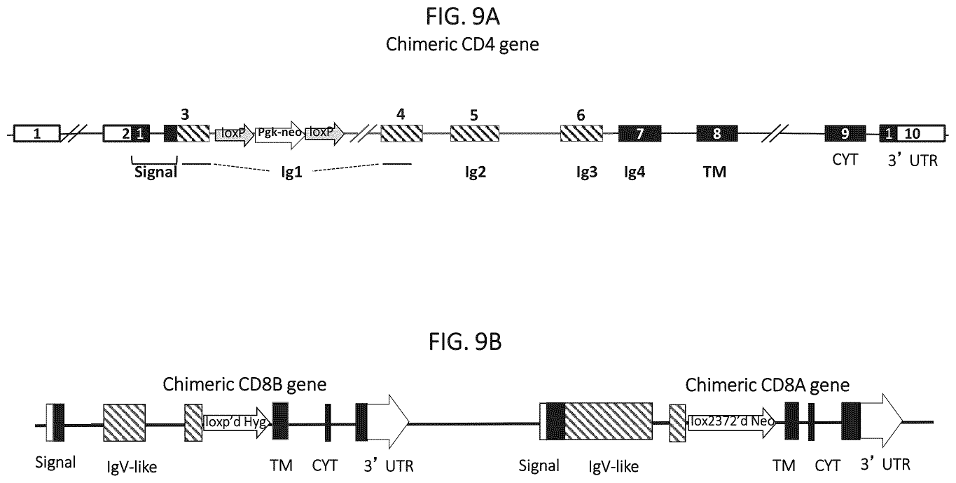

Human CD4 gene is localized to chromosome 12, and is thought to contain 10 exons. CD4 gene encodes a protein with amino-terminal hydrophobic signal sequence, encoded by exons 2 and 3 of the gene. The protein comprises four extracellular immunoglobulin-like domains, Ig1-Ig4, also commonly and respectively referred to as D1-D4 domains. Maddon et al. (1987) Structure and expression of the human and mouse T4 genes, Proc. Natl. Acad. Sci. USA 84:9155-59. D1 domain is believed to be encoded by exon 3 (sequence downstream of signal peptide) and exon 4, while D2, D3, and D4 are encoded by a separate exon each—exons 5, 6, and 7, respectively (see FIG. 9A: D1, D2, D3 and D4 domains are encoded by sequences designated as Ig1, Ig2, Ig3 and Ig4, respectively). Littman (1987) The Structure of the CD4 and CD8 Genes, Ann. Rev. Immunol. 5:561-84; Hanna et al. (1994) Specific Expression of the Human CD4 Gene in Mature CD4+CD8− and Immature CD4+CD8+ T cells and in Macrophages of Transgenic Mice, Mol. Cell. Biol. 14(2):1084-94; Maddon et al., supra. At areas of high protein concentration, such as the area of contact between T cell and antigen-presenting cell, the molecule tends to homodimerize through interactions between opposing D4 domains. Zamoyska (1998) CD4 and CD8: modulators of T cell receptor recognition of antigen and of immune responses? Curr. Opin. Immunol. 10:82-87; Wu et al. (1997) Dimeric association and segmental variability in the structure of human CD4, Nature 387:527; Moldovan et al. (2002) CD4 Dimers Constitute the Functional Component Required for T Cell Activation, J. Immunol. 169:6261-68.

D1 domain of CD4 resembles immunoglobulin variable (V) domain, and, together with a portion of D2 domain, is believed to bind (associate with) MHC II, e.g., at an MHC II co-receptor binding site. Huang et al. (1997) Analysis of the contact sites on the CD4 Molecule with Class II MHC Molecule, J. Immunol. 158:216-25. In turn, MHC II interacts with T cell co-receptor CD4 at the hydrophobic crevice at the junction between MHC II α2 and β2 domains. Wang and Reinherz (2002) Structural Basis of T Cell Recognition of Peptides Bound to MHC Molecules, Molecular Immunology, 38:1039-49.

Domains D3 and D4 of the CD4 co-receptor are believed to interact with the TCR-CD3 complex as the substitution of these two domains abrogated the ability of CD4 to bind to TCR. Vignali et al. (1996) The Two Membrane Proximal Domains of CD4 Interact with the T Cell Receptor, J. Exp. Med. 183:2097-2107. CD4 molecule exists as a dimer, and residues in the D4 domain of the molecule are believed to be responsible for CD4 dimerization. Moldovan et al. (2002) CD4 Dimers Constitute the Functional Components Required for T Cell Activation, J. Immunol. 169:6261-68.

Exon 8 of the CD4 gene encodes the transmembrane domain, while the remainder of the gene encodes the cytoplasmic domain. CD4 cytoplasmic domain possesses many distinct functions. For example, the cytoplasmic domain of CD4 recruits a tyrosine kinase Lck. Lck is a Src family kinase that is associated with CD4 and CD8 cytoplasmic domains and simultaneous binding of the co-receptors and TCRs to the same MHC leads to increased tyrosine phosphorylation of CD3 and □ chain of the TCR complex, which in turn leads to recruitment of other factors that play a role in T cell activation. Itano and colleagues have proposed that cytoplasmic tail of CD4 also promotes differentiation of CD4+CD8+ T cells into CD4+ lineage by designing and testing expression of hybrid protein comprising CD8 extracellular domain and CD4 cytoplasmic tail in transgenic mice. Itano et al. (1996) The Cytoplasmic Domain of CD4 Promotes the Development of CD4 Lineage T Cells, J. Exp. Med. 183:731-41. The expression of the hybrid protein led to the development of MHC I-specific, CD4 lineage T cells. Id.

CD4 co-receptor appears to be the primary receptor for HIV virus, with the CD4⁺ T cell depletion being an indicator of disease progression. The cytoplasmic tail of CD4 appears to be essential for delivering apoptotic signal to CD4+ T cells in HIV-induced apoptosis. Specifically, the interaction of CD4 and Lck was shown to potentiate HIV-induced apoptosis in these cells. Corbeil et al. (1996) HIV-induced Apoptosis Requires the CD4 Receptor Cytoplasmic Tail and Is Accelerated by Interaction of CD4 with p561ck, J. Exp. Med. 183:39-48.

T cells develop in the thymus progressing from immature CD4−/CD8− (double negative or DN) thymocytes to CD4+/CD8+(double positive or DP) thymocytes, which eventually undergo positive selection to become either CD4+ or CD8+(single positive or SP) T cells. Dβ thymocytes that receive signals through MHC I-restricted TCR differentiate into CD8+ T cells, while Dβ thymocytes that receive signals through MHC II-restricted TCR differentiate into CD4+ T cells. The cues received by the Dβ cell that lead to its differentiation into either CD4+ of CD8+ T cell have been a subject of much research. Various models for CD4/CD8 lineage choice have been proposed and are reviewed in Singer et al. (2008) Lineage fate and intense debate: myths, models and mechanisms of CD4− versus CD8− lineage choice, Nat. Rev. Immunol. 8:788-801.

Deactivation of a specific T cell co-receptor as a result of positive selection is a product of transcriptional regulation. For CD4, it has been shown that an enhancer located 13 kb upstream of exon 1 of CD4 upregulates CD4 expression in CD4+ and CD8+ T cells. Killeen et al. (1993) Regulated expression of human CD4 rescues helper T cell development in mice lacking expression of endogenous CD4, EMBO J. 12:1547-53. A cis-acting transcriptional silencer located within the first intron of murine CD4 gene functions to silence expression of CD4 in cells other than CD4+ T cells. Siu et al. (1994) A transcriptional silencer control the developmental expression of the CD4 gene, EMBO J. 13:3570-3579.

Because important transcriptional regulators (e.g., promoters, enhancers, silencers, etc.) that control CD4 lineage choice were missing in several strains of previously developed transgenic mice expressing human CD4, these mice were not able to recapitulate normal T cell lineage development, and produced immune cells other than CD4+ T cells that expressed CD4. See, e.g., Law et al. (1994) Human CD4 Restores Normal T Cell Development and Function in Mice Deficient in CD4, J. Exp. Med. 179:1233-42 (CD4 expression in CD8+ T cells and B cells); Fugger et al. (1994) Expression of HLA-DR4 and human CD4 transgenes in mice determines the variable region β-chain T-cell repertoire and mediates an HLA-D-restricted immune response, Proc. Natl. Acad. Sci. USA, 91:6151-55 (CD4 expressed on all CD3⁺ thymocytes and B cells). Thus, in one embodiment, there may be a benefit in developing a genetically modified animal that retains endogenous mouse promoter and/or other regulatory elements in order for the animal to produce T cells that are capable of undergoing T cell development and lineage choice.

Thus, in various embodiments, the invention provides a genetically modified non-human animal, comprising, e.g., at its endogenous T cell co-receptor locus (e.g., CD4 locus), a nucleotide sequence encoding a chimeric human/non-human T cell co-receptor polypeptide. In one embodiment, a human portion of the chimeric polypeptide comprises all or substantially all of an extracellular portion (or part thereof, e.g., one or more extracellular domains, e.g., at least two consecutive extracellular domains) of a human T cell co-receptor. In one embodiment, a non-human portion of the chimeric polypeptide comprises transmembrane and cytoplasmic domains of a non-human T cell co-receptor. In one embodiment, the non-human animal expresses a functional chimeric T cell co-receptor polypeptide. Thus, in one aspect, the invention provides a genetically modified non-human animal comprising at its endogenous CD4 locus a nucleotide sequence encoding a chimeric human/non-human CD4 polypeptide, wherein a human portion of the chimeric polypeptide comprises all or substantially all of an extracellular portion of a human CD4, wherein a non-human portion comprises at least transmembrane and cytoplasmic domains of a non-human CD4, and wherein the animal expresses a functional chimeric CD4 polypeptide. In one aspect, the non-human animal only expresses the humanized CD4 polypeptide, i.e., chimeric human/non-human CD4 polypeptide, and does not express a functional endogenous non-human CD4 protein from its endogenous CD4 locus.

In one embodiment, the human portion of the chimeric human/non-human CD4 polypeptide comprises all or substantially all of the extracellular portion of a human CD4 polypeptide. In another embodiment, the human portion of the chimeric human/non-human CD4 polypeptide comprises at least all or substantially all of the MHC II binding domain of the human CD4 polypeptide (e.g., a substantial portion of human D1 and D2 domains); in one embodiment, the human portion of the chimeric human/non-human CD4 polypeptide comprises all or substantially all of D1, D2, and D3 domains of the human CD4 polypeptide; in yet another embodiment, the human portion of the chimeric human/non-human CD4 polypeptide comprises all or substantially all of immunoglobulin-like domains of CD4, e.g., domains termed D1, D2, D3, and D4. In yet another embodiment, the human portion of the chimeric human/non-human CD4 polypeptide comprises in its human portion all or substantially all of the human CD4 sequence that is responsible for interacting with MHC II and/or extracellular portion of a T cell receptor. In yet another embodiment, the human portion of the chimeric human/non-human CD4 polypeptide comprises all or substantially all of the extracellular portion of the human CD4 that is responsible for interacting with MHC II and/or the variable domain of a T cell receptor. Therefore, in one embodiment, the nucleotide sequence encoding the human portion of the chimeric CD4 polypeptide comprises all or substantially all of the coding sequence of domains D1-D2 of the human CD4 (e.g., a portion of exon 3 and exons 4-5 of the human CD4 gene); in another embodiment, it comprises all or substantially all of the coding sequence of D1-D3 of the human CD4 (e.g., portion of exon 3 and exons 4-6 of the human CD4). Thus, in one embodiment, the nucleotide sequence encoding chimeric human/non-human CD4 comprises nucleotide sequences encoding all or substantially all D1-D3 domains of the human CD4. In another embodiment, the nucleotide sequence encoding the human portion of the chimeric CD4 polypeptide comprises the coding sequence of D1-D4 domains of the human CD4 gene. In another embodiment, the nucleotide sequence may comprise the nucleotide sequence encoding mouse CD4 signal peptide, e.g., region encoded by portions of exons 2-3 of the mouse gene. In another embodiment, the nucleotide sequence may comprise the nucleotide sequence encoding a human CD4 signal peptide. In one embodiment, the chimeric human/non-human CD4 polypeptide comprises an amino acid sequence set forth in SEQ ID NO:1, and the human portion of the chimeric polypeptide spans about amino acids 27-319 of SEQ ID NO:1 (set forth separately in SEQ ID NO:2).

In one embodiment, the non-human animal expresses a chimeric human/non-human CD4 polypeptide sequence. In one embodiment, a human portion of the chimeric CD4 sequence comprises one or more conservative or non-conservative modifications.

In one aspect, a non-human animal that expresses a human CD4 sequence is provided, wherein the human CD4 sequence is at least about 85%, 90%, 95%, 96%, 97%, 98%, or 99% identical to a human CD4 sequence. In a specific embodiment, the human CD4 sequence is at least about 90%, 95%, 96%, 97%, 98%, or 99% identical to the human CD4 sequence described in U.S. Pat. No. 10,820,581. In one embodiment, the human CD4 sequence comprises one or more conservative substitutions. In one embodiment, the human CD4 sequence comprises one or more non-conservative substitutions.

In some embodiments, a portion, e.g., a human portion of the chimeric CD4, may comprise substantially all of the sequence indicated herein (e.g., substantially all of a protein domain indicated herein). Substantially all sequence generally includes 85%, 90%, 95%, 96%, 97%, 98%, or 99% of the amino acids believed to represent a particular portion of the protein (e.g., a particular functional domain, etc.). One skilled in the art would understand that the boundaries of a functional domain may vary slightly depending on the alignment and domain prediction methods used.

In one aspect, the non-human portion of the chimeric human/non-human CD4 polypeptide comprises at least transmembrane and cytoplasmic domains of the non-human CD4 polypeptide. Due to the important functions served by CD4 cytoplasmic domain, retention of the endogenous non-human (e.g., mouse) sequence in genetically engineered animals ensures preservation of proper intracellular signaling and other functions of the co-receptor. In one embodiment, the non-human animal is a mouse, and the non-human CD4 polypeptide is a mouse CD4 polypeptide. Although a specific mouse CD4 sequence is described in U.S. Pat. No. 10,820,581, cited in the Examples, any suitable sequence derived therefrom, e.g., sequence comprising conservative/non-conservative amino acid substitutions, is encompassed herein. In one embodiment, the non-human portion of the chimeric CD4 co-receptor comprises any sequence of the endogenous CD4 that has not been humanized.

The non-human animal described herein may comprise at its endogenous locus a nucleotide sequence encoding a chimeric human/non-human CD4 polypeptide. In one aspect, this results in a replacement of a portion of an endogenous CD4 gene with a nucleotide sequence encoding a portion of a human CD4 polypeptide. In one embodiment, such replacement is a replacement of endogenous nucleotide sequence encoding, e.g., all or substantially all of the extracellular domain of a non-human CD4, e.g., a sequence encoding at least all or substantially all of the first immunoglobulin-like domain (i.e., D1) of a non-human CD4 (e.g., a sequence encoding all or substantially all of domains D1-D2 of a non-human CD4, e.g., a sequence encoding all or substantially all of domains D1-D3 of a non-human CD4, e.g., a sequence encoding all or substantially all of domains D1-D4 of a non-human CD4), with a human nucleotide sequence encoding the same. In one embodiment, the replacement results in a chimeric protein comprising human CD4 sequence that is responsible for interacting with MHC II and/or extracellular portion of a T cell receptor. In yet another embodiment, the replacement results in a chimeric protein comprising human CD4 sequence that is responsible for interacting with MHC II and/or variable domain of a T cell receptor. In one embodiment, the replacement does not comprise a replacement of a CD4 sequence encoding at least transmembrane and cytoplasmic domains of a non-human CD4 polypeptide. Thus, in one aspect, the non-human animal expresses a chimeric human/non-human CD4 polypeptide from the endogenous non-human CD4 locus. In yet another embodiment, the replacement results in a protein comprising a polypeptide sequence set forth in SEQ ID NO:1.

In one embodiment, the nucleotide sequence of the chimeric human/non-human CD4 locus (e.g., chimeric human/rodent CD4 locus, e.g., chimeric human/mouse CD4 locus) described herein is provided. In one aspect, because the chimeric human/non-human (e.g., human/rodent, e.g., human/mouse) CD4 sequence is placed at the endogenous non-human (e.g., rodent, e.g., mouse) CD4 locus, it retains the CD4 enhancer element located upstream of the first CD4 exon. In one embodiment, the replacement at the endogenous non-human (e.g., rodent, e.g., mouse) CD4 locus comprises a replacement of, e.g., a portion of exon 3 encoding D1, and exons 4-6 encoding the rest of D1 and D2-D3 of CD4 polypeptide; thus, in one aspect, the chimeric CD4 locus retains the cis-acting silencer located in intron 1 of the non-human (e.g., mouse) CD4 gene. Thus, in one embodiment, the chimeric locus retains endogenous non-human (e.g., rodent, e.g., mouse) CD4 promoter and regulatory elements. In another embodiment, the chimeric locus may contain human promoter and regulatory elements to the extent those allow proper CD4 expression, CD4+ T cell development, CD4 lineage choice, and co-receptor function. Thus, in some aspects, the animals of the invention comprise a genetic modification that does not alter proper lineage choice and development of T cells. In one aspect, the animals (e.g., rodents, e.g., mice) of the invention do not express chimeric CD4 polypeptide on immune cells other than cells that normally express CD4. In one aspect, animals do not express CD4 on B cells or mature CD8+ T cells. In one embodiment, the replacement results in retention of elements that allow proper spatial and temporal regulation of CD4 expression.

In various embodiments, a non-human animal (e.g., a rodent, e.g., a mouse or rat) that expresses a functional chimeric CD4 protein from a chimeric CD4 locus as described herein displays the chimeric protein on a cell surface, e.g., T cell surface. In one embodiment, the non-human animal expresses the chimeric CD4 protein on a cell surface in a cellular distribution that is the same as observed in a human. In one aspect, the CD4 protein of the invention is capable of interacting with an MHC II protein expressed on the surface of a second cell, e.g., an antigen presenting cell (APC).

In various embodiments, the invention generally provides genetically modified non-human animals that comprise in their genome, e.g., at an endogenous CD8 locus, a nucleotide sequence encoding a human or humanized CD8 polypeptide; thus, the animals express a human or humanized CD8 polypeptide. In various embodiments, the invention provides non-human animals that comprise in their genome, e.g., at an endogenous CD8 locus, a nucleotide sequence encoding a human or humanized CD8α polypeptide and/or a nucleotide sequence encoding a human or humanized CD8β polypeptide. Thus, the genetically modified non-human animal of the invention expresses a human or humanized CD8α and/or a human or humanized CD8β polypeptide(s).

Human CD8 protein is typically expressed on cell surface as heterodimer of two polypeptides, CD8α and CD8β, although disulfide-linked homodimers and homomultimers have also been detected (e.g., in NK cells and intestinal γδ T cells, which express CD8aa). The genes encoding human CD8α and CD8β are located in close proximity to each other on chromosome 2. Nakayama et al. (1992) Recent Duplication of the Two Human CD8 β-chain genes, J. Immunol. 148:1919-27. CD8α protein contains a leader peptide, an immunoglobulin V-like region, a hinge region, a transmembrane domain and a cytoplasmic tail. Norment et al. (1989) Alternatively Spliced mRNA Encodes a Secreted Form of Human CD8α. Characterization of the Human CD8α gene, J. Immunol. 142:3312-19. The exons/introns of the CD8α gene are depicted schematically in FIG. 9B.

Human CD8β gene lies upstream of the CD8α gene on chromosome 2. Multiple isoforms generated by alternative splicing of CD8β gene have been reported, with one isoform predicted to lack a transmembrane domain and generate a secreted protein. Norment et al. (1988) A second subunit of CD8 is expressed in human T cells, EMBO J. 7:3433-39. The exons/introns of CD8β gene are also depicted schematically in FIG. 9B.

The membrane-bound CD8β protein contains an N-terminal signal sequence, followed by immunoglobulin V-like domain, a short extracellular hinge region, a transmembrane domain, and a cytoplasmic tail. See, Littman (1987) The structure of the CD4 and CD8 genes, Ann Rev. Immunol. 5:561-84. The hinge region is a site of extensive glycosylation, which is thought to maintain its conformation and protect the protein from cleavage by proteases. Leahy (1995) A structural view of CD4 and CD8, FASEB J. 9:17-25.

CD8 protein is commonly expressed on cytotoxic T cells, and interacts with MHC I molecules. The interaction is mediated through CD8 binding to the α3 domain of MHC I. Although binding of MHC class I to CD8 is about 100-fold weaker than binding of TCR to MHC class I, CD8 binding enhances the affinity of TCR binding. Wooldridge et al. (2010) MHC Class I Molecules with Superenhanced CD8 Binding Properties Bypass the Requirement for Cognate TCR Recognition and Nonspecifically Activate CTLs, J. Immunol. 184:3357-3366.

CD8 binding to MHC class I molecules is species-specific; the mouse homolog of CD8, Lyt-2, was shown to bind H-2D^{d} molecules at the α3 domain, but it did not bind HLA-A molecules. Connolly et al. (1988) The Lyt-2 Molecule Recognizes Residues in the Class I α3 Domain in Allogeneic Cytotoxic T Cell Responses, J. Exp. Med. 168:325-341. Differential binding was presumably due to CDR-like determinants (CDR1- and CDR2-like) on CD8 that were not conserved between humans and mice. Sanders et al. (1991) Mutations in CD8 that Affect Interactions with HLA Class I and Monoclonal Anti-CD8 Antibodies, J. Exp. Med. 174:371-379; Vitiello et al. (1991) Analysis of the HLA-restricted Influenza-specific Cytotoxic T Lymphocyte Response in Transgenic Mice Carrying a Chimeric Human-Mouse Class I Major Histocompatibility Complex, J. Exp. Med. 173:1007-1015; and, Gao et al. (1997) Crystal structure of the complex between human CD8α and HLA-A2, Nature 387:630-634. It has been reported that CD8 binds HLA-A2 in a conserved region of the α3 domain (at position 223-229). A single substitution (V245A) in HLA-A reduced binding of CD8 to HLA-A, with a concomitant large reduction in T cell-mediated lysis. Salter et al. (1989), Polymorphism in the α3 domain of HLA-A molecules affects binding to CD8, Nature 338:345-348. In general, polymorphism in the α3 domain of HLA-A molecules also affected binding to CD8. Id. In mice, amino acid substitution at residue 227 in H-2D^{d} affected the binding of mouse Lyt-2 to H-2D^{d}, and cells transfected with a mutant H-2D^{d} were not lysed by CD8⁺ T cells. Potter et al. (1989) Substitution at residue 227 of H-2 class I molecules abrogates recognition by CD8-dependent, but not CD8-independent, cytotoxic T lymphocytes, Nature 337:73-75. Thus, expression of human or humanized CD8 may be beneficial for studying T cell responses to antigen presented by human or humanized MHC I.

Similarly to CD4, the cytoplasmic domain of CD8 interacts with tyrosine kinase Lck, which in turn leads to T cell activation. Although Lck seems to interact with the cytoplasmic domain of CD8α, it appears that this interaction is regulated by the presence of the cytoplasmic domain of CD8β because mutations or deletion of CD8β cytoplasmic domain resulted in reduced CD8α-associated Lck activity. Irie et al. (1998) The cytoplasmic domain of CD8β Regulates Lck Kinase Activation and CD8 T cell Development, J. Immunol. 161:183-91. The reduction in Lck activity was associated with impairment in T cell development. Id.

Expression of CD8 on appropriate cells, e.g., cytotoxic T cells, is tightly regulated by a variety of enhancer elements located throughout the CD8 locus. For instance, at least 4 regions of DNAse I-hypersensitivity, regions often associated with regulator binding, have been identified at the CD8 locus. Hosert et al. (1997) A CD8 genomic fragment that directs subset-specific expression of CD8 in transgenic mice, J. Immunol. 158:4270-81. Since the discovery of these DNAse I-hypersensitive regions at CD8 locus, at least 5 enhancer elements have been identified, spread throughout the CD8 locus, that regulate expression of CD8α and/or β in T cells of various lineages, including DP, CD8 SP T cells, or cells expressing γδ TCR. See, e.g., Kioussis et al. (2002) Chromatin and CD4, CD8A, and CD8B gene expression during thymic differentiation, Nature Rev. 2:909-919 and Online Erratum; Ellmeier et al. (1998) Multiple Development Stage-Specific Enhancers Regulate CD8 Expression in Developing Thymocytes and in Thymus-Independent T cells, Immunity 9:485-96.

Thus, similarly to the benefit derived from retaining endogenous CD4 promoter and regulatory elements for human or humanized CD4 genetically modified animals, in some embodiments, there may be a benefit in developing a genetically modified non-human animal that retains endogenous mouse promoter and regulatory elements that would control expression of human or humanized CD8. There may be a particular benefit in creating genetically modified animals comprising a replacement of endogenous non-human sequences encoding CD8α and/or proteins with those encoding human or humanized CD8α and/or R proteins, as described herein.

In various embodiments, the invention provides a genetically modified non-human animal comprising in its genome, e.g., at its endogenous CD8 locus, at least one nucleotide sequence encoding a chimeric human/non-human CD8 polypeptide (e.g., CD8α and/or β polypeptide), wherein a human portion of the polypeptide comprises all or substantially all of an extracellular portion (or a part thereof, e.g., an extracellular domain) of a human CD8 polypeptide (e.g., CD8α and/or β), wherein a non-human portion comprises at least transmembrane and cytoplasmic domains of a non-human CD8 (e.g., CD8α and/or β), and wherein the animal expresses the chimeric CD8 polypeptide (e.g., CD8α and/or β polypeptide). Thus, in one embodiment, the invention provides a genetically modified non-human animal comprising at its endogenous non-human CD8 locus a first nucleotide sequence encoding a chimeric human/non-human CD8α polypeptide and a second nucleotide sequence encoding a chimeric human/non-human CD8β polypeptide, wherein the first nucleotide sequence comprises a sequence that encodes all or substantially all of the extracellular portion of a human CD8α polypeptide and at least transmembrane and cytoplasmic domains of a non-human CD8α polypeptide, and wherein the second nucleotide sequence comprises a sequence that encodes all or substantially all of the extracellular portion of a human CD8β polypeptide and at least transmembrane and cytoplasmic domains of a non-human CD8β polypeptide, wherein the animal expresses a functional chimeric human/non-human CD8 protein. In one aspect, the non-human animal only expresses a humanized CD8 polypeptide (e.g., chimeric human/non-human CD8α and/or R polypeptide) and does not express a corresponding functional non-human CD8 polypeptide(s) from the endogenous CD8 locus.

In one embodiment, the chimeric human/non-human CD8α polypeptide comprises in its human portion all or substantially all of the extracellular portion of a human CD8α polypeptide. In one embodiment, the human portion of the chimeric CD8α polypeptide comprises at least the MHC I binding domain of the human CD8α polypeptide. In one embodiment, the human portion of the chimeric CD8α polypeptide comprises the sequence of at least all or substantially all of the immunoglobulin V-like domain of the human CD8α. In one embodiment, the nucleotide sequence encoding the human portion of the chimeric CD8α polypeptide comprises at least the exons that encode an extracellular portion of the human CD8α polypeptide. In one embodiment, the nucleotide sequence comprises at least the exons that encode the Ig V-like domains. In one embodiment, the extracellular portion of a human CD8α polypeptide is a region encompassing the portion of the polypeptide that is not transmembrane or cytoplasmic domain. In one embodiment, the nucleotide sequence encoding the chimeric human/non-human CD8α polypeptide comprises the sequence encoding a non-human (e.g., rodent, e.g., mouse) CD8α signal peptide. Alternatively, the nucleotide sequence may comprise the sequence encoding a human CD8α signal sequence. In one embodiment, the chimeric human/non-human CD8α polypeptide comprises an amino acid sequence set forth in SEQ ID NO:3, and the human portion of the chimeric polypeptide is set forth at amino acids 28-179 of SEQ ID NO:3 (represented separately in SEQ ID NO:4).

Similarly, in one embodiment, the chimeric human/non-human CD8β polypeptide comprises in its human portion all or substantially all of the extracellular portion of a human CD8β polypeptide. In one embodiment, the human portion of the chimeric CD8β polypeptide comprises the sequence of all or substantially all of the immunoglobulin V-like domain of human CD80. In one embodiment, the nucleotide sequence encoding the human portion of the chimeric CD8β polypeptide comprises at least the exons that encode the extracellular portion of the human CD8β polypeptide. In one embodiment, the nucleotide sequence encoding the human portion of the chimeric human/non-human CD8β polypeptide comprises at least the exons that encode the IgG V-like domain of human CD80. In one embodiment, the nucleotide sequence encoding the chimeric human/non-human CD8β polypeptide comprises the sequence encoding a non-human (e.g., rodent, e.g., mouse) CD8β signal peptide. Alternatively, the nucleotide sequence may comprise the sequence encoding a human CD8β signal sequence. In one embodiment, the chimeric human/non-human CD8β polypeptide comprises an amino acid sequence set forth in SEQ ID NO:5, and the human portion of the chimeric polypeptide is set forth at amino acids 15-165 of SEQ ID NO:5 (represented separately in SEQ ID NO:6).

In one embodiment, the non-human animal expresses a chimeric human/non-human CD8α and/or CD8β polypeptides. In some embodiments, the human portion of the chimeric human/non-human CD8α and/or β polypeptide comprises one or more conservative or nonconservative modification(s).

In one aspect, a non-human animal that expresses a human CD8α and/or β polypeptide sequence is provided, wherein the human CD8α and/or β polypeptide sequence is at least about 85%, 90%, 95%, 96%, 97%, 98%, or 99% identical to a human CD8α and/or β polypeptide sequence, respectively. In a specific embodiment, the human CD8α and/or β polypeptide sequence is at least about 90%, 95%, 96%, 97%, 98%, or 99% identical to the respective human CD8α and/or β polypeptide sequence described in U.S. Pat. No. 9,848,587. In one embodiment, the human CD8α and/or β polypeptide sequence comprises one or more conservative substitutions. In one embodiment, the human CD8α and/or β polypeptide sequence comprises one or more non-conservative substitutions.

In some embodiments, a portion, e.g., a human portion of the chimeric CD8, may comprise substantially all of the sequence indicated herein (e.g., substantially all of a protein domain indicated herein). Substantially all sequence generally includes 85%, 90%, 95%, 96%, 97%, 98%, or 99% of the amino acids believed to represent a particular portion of the protein (e.g., a particular functional domain, etc.). One skilled in the art would understand that the boundaries of a functional domain may vary slightly depending on the alignment and domain prediction methods used.

In one aspect, the non-human portion of the chimeric human/non-human CD8α and/or β polypeptide comprises at least transmembrane and/or cytoplasmic domain of the non-human CD8α and/or β polypeptide, respectively. Due to the important functions served by CD8 cytoplasmic domain, retention of the endogenous non-human (e.g., mouse) sequence in genetically engineered animals ensures preservation of proper intracellular signaling and other functions of the co-receptor. In one embodiment, the non-human animal is a mouse, and the non-human CD8α and/or β polypeptide is a mouse CD8α and/or β polypeptide, respectively. Although specific mouse CD8α and β sequences are described in U.S. Pat. No. 9,848,587, cited in in the Examples, any suitable sequence derived therefrom, e.g., sequence comprising conservative/non-conservative amino acid substitutions, is encompassed herein. In one embodiment, the non-human animal (e.g., rodent, e.g., mouse) retains any endogenous sequence that has not been humanized.

The non-human animal described herein may comprise at its endogenous locus a nucleotide sequence encoding a chimeric human/non-human CD8α and/or β polypeptide. In one aspect, this results in a replacement of a portion of an endogenous CD8α gene with a nucleotide sequence encoding a portion of a human CD8α polypeptide, and/or a replacement of a portion of an endogenous CD8β gene with a nucleotide sequence encoding a portion of a human CD8β polypeptide. In one embodiment, such replacement is a replacement of endogenous nucleotide sequence encoding all or substantially all of extracellular portion of a non-human CD8α and/or β with a human nucleotide with a human nucleotide sequence encoding the same. In one embodiment, such replacement is a replacement of a sequence encoding at least all or substantially all of the immunoglobulin V-like domain of a non-human CD8α and/or β with a human nucleotide sequence encoding the same. In one embodiment, the replacement does not comprise a replacement of a CD8α and/or β sequence encoding transmembrane and cytoplasmic domain of a non-human CD8α and/or β polypeptide. Thus, the non-human animal expresses a chimeric human/non-human CD8α and/or β polypeptide from the endogenous non-human CD8 locus. In yet another embodiment, the replacement results in a CD8α and/or β protein comprising a polypeptide sequence set forth in SEQ ID NO:3 and/or 5, respectively.

In one embodiment, the nucleotide sequence of the chimeric human/non-human CD8 locus (e.g., chimeric rodent CD8 locus, e.g., chimeric mouse CD8 locus) is provided. In one aspect, because the chimeric human/non-human (e.g., human/rodent, e.g., human/mouse) CD8α and/or β sequence is placed at respective endogenous non-human (e.g., rodent, e.g., mouse) CD8α and/or β locus, it retains endogenous CD8α and/or β promoter and regulatory elements. In another embodiment, the chimeric locus may contain human CD8α and/or β promoter and regulatory elements to the extent those allow proper CD8α and/or β expression (proper spatial and temporal protein expression), CD8+ T cell development, CD8 lineage choice, and co-receptor function. Thus, in one aspect, the animals of the invention comprise a genetic modification that does not alter proper lineage choice and development of T cells. In one aspect, the animals (e.g., rodents, e.g., mice) of the invention do not express chimeric CD8 protein on immune cells other than cells that normally express CD8, e.g., animals do not express CD8 on B cells or mature CD4+ T cells. In one embodiment, the replacement results in retention of elements that allow proper spatial and temporal regulation of CD8α and/or β expression.

In various embodiments, a non-human animal (e.g., a rodent, e.g., a mouse or rat) that expresses a functional chimeric CD8 protein (e.g., CD8α(3 or CD8aa) from a chimeric CD8 locus as described herein displays the chimeric protein on a cell surface. In one embodiment, the non-human animal expresses the chimeric CD8 protein on a cell surface in a cellular distribution that is the same as observed in a human. In one aspect, the CD8 protein of the invention is capable of interacting with an MHC I protein expressed on the surface of a second cell.

### Human or Humanized MHC Molecules

Although recognition of antigen by γ/δ T cells may not require interactions between T cell co-receptors (e.g., CD4 and CD8) and MHC, since a non-human animal as described herein may comprise, in addition to human or humanized TRD (and TRG) loci, human or humanized TRA, TRB, CD4 and/or CD8 (e.g., CD8α and CD8β) loci, a non-human animal as described herein may further comprise one or more human or humanized MHC loci.

In various embodiments, provided herein are genetically modified non-human animals that co-express at least one humanized T cell co-receptor, at least one humanized MHC that associates with the humanized T cell co-receptor, and a human or humanized α/β TCR, which upon recognizing and binding peptide presented by the humanized MHC, and in conjunction with the humanized co-receptor, provides activation signals to the cell expressing the humanized TCR and chimeric T cell co-receptor polypeptides. Accordingly, a non-human animal as disclosed herein comprises at least one of a first, second, and/or third nucleic acid sequence, each of which encodes a different human or humanized MHC polypeptide selected from the group consisting of a human or humanized MHC II α polypeptide, a human or humanized MHC II β polypeptide, and a human or humanized MHC I α polypeptide; the non-human animal also optionally comprises a human or humanized β2 microglobulin. Use of the first, second, and third designations herein is not to be construed as limiting the non-human animals disclosed herein as requiring all three nucleic acid sequences or the presence of any of the human or humanized MHC polypeptides in any specific order.

Accordingly, in some embodiments, a non-human animal as disclosed herein may comprise, e.g., a first and second nucleotide sequence encoding e.g., a human or chimeric CD8α polypeptide and a human or chimeric CD8β polypeptide, an unrearranged T cell receptor (TCR) a variable gene locus comprising at least one human Vα segment and at least one human Jα segment, operably linked to a non-human TCRα constant region gene sequence and/or an unrearranged TCRβ variable gene locus comprising at least one human Vβ segment, at least one human Dβ segment, and at least one human Jβ segment, operably linked to a non-human TCRβ constant region gene sequence, and optionally a first and second nucleic acid sequence encoding, e.g., a human or humanized MHC I α polypeptide and a human or humanized β2-microglobulin polypeptide. In other embodiments, a non-human animal as disclosed herein may comprise, e.g., a first nucleotide sequence encoding, e.g., a chimeric CD4 polypeptide; an unrearranged T cell receptor (TCR) a variable gene locus comprising at least one human Vα segment and at least one human Jα segment, operably linked to a non-human TCRα constant region gene sequence and/or an unrearranged TCRβ variable gene locus comprising at least one human Vβ segment, at least one human Dβ segment, and at least one human Jβ segment, operably linked to a non-human TCRβ constant region gene sequence; and optionally a first and second nucleic acid sequence encoding, e.g., a human or humanized MHC II α polypeptide and a human or humanized MHC II polypeptide. In some embodiment, a non-human animal as disclosed herein may comprise, e.g., a first, second and third nucleotide sequence encoding e.g., a chimeric CD4 polypeptide, a chimeric CD8α polypeptide, and a chimeric CD8β polypeptide; an unrearranged T cell receptor (TCR) a variable gene locus comprising at least one human Vα segment and at least one human Jα segment, operably linked to a non-human TCRα constant region gene sequence and/or an unrearranged TCRβ variable gene locus comprising at least one human Vβ segment, at least one human Dβ segment, and at least one human Jβ segment, operably linked to a non-human TCRβ constant region gene sequence; and optionally a first, second, third and fourth nucleic acid sequence encoding, e.g., a human or humanized MHC II α polypeptide, a human or humanized MHC II β polypeptide, a human or humanized MHC I α polypeptide, and a human or humanized β2-microglobulin polypeptide.

In various embodiments, provided herein is a genetically modified non-human animal, e.g., rodent (e.g., mouse or rat) comprising in its genome a nucleic acid sequence encoding a human or humanized MHC I polypeptide and/or a nucleic acid sequence encoding human or humanized MHC II protein. The MHC I nucleic acid sequence may encode an MHC I polypeptide that is partially human and partially non-human, e.g., chimeric human/non-human MHC I polypeptide, and the MHC II nucleic acid sequence may encode an MHC II protein that is partially human and partially non-human, e.g., chimeric human/non-human MHC II protein (e.g., comprising chimeric human/non-human MHC II α and β polypeptides). In some aspects, the animal does not express endogenous MHC I and/or endogenous MHC II polypeptides, e.g., functional endogenous MHC I and/or MHC II polypeptides on a cell surface. In some embodiments, the only MHC I and/or MHC II molecules expressed on a cell surface of the animal are chimeric MHC I and/or MHC II molecules.

A genetically modified non-human animal comprising in its genome, e.g., at the endogenous locus, a nucleic acid sequence encoding a chimeric human/non-human MHC I polypeptide is disclosed in U.S. Pat. Nos. 9,615,550 and 9,591,835; each of which are incorporated herein by reference in their entireties. A genetically modified non-human animal comprising in its genome, e.g., at the endogenous locus, a nucleic acid sequence encoding humanized, e.g., chimeric human/non-human MHC II polypeptides is disclosed in U.S. Pat. No. 8,847,005 and in U.S. Pat. No. 9,043,966, each of which are incorporated herein by reference in their entireties. A genetically modified non-human animal comprising in its genome, e.g., at the endogenous locus, a nucleic acid sequence encoding a chimeric human/non-human MHC I polypeptide and comprising in its genome, e.g., at the endogenous locus, a nucleic acid sequence encoding humanized, e.g., chimeric human/non-human MHC II polypeptides, is disclosed in U.S. Patent Publication No. 20140245467, which is incorporated herein by reference in its entirety.

In various embodiments provided herein is a genetically modified non-human animal comprising in its genome, e.g., at one or more endogenous MHC loci, a first nucleic acid sequence encoding a chimeric human/non-human MHC I polypeptide, wherein a human portion of the chimeric MHC I polypeptide comprises an extracellular portion (or part thereof, e.g., one or more extracellular domains) of a human MHC I polypeptide; a second nucleic acid sequence encoding a chimeric human/non-human MHC II α polypeptide, wherein a human portion of the chimeric MHC II α polypeptide comprises an extracellular portion (or part thereof, e.g., one or more extracellular domains) of a human MHC II α polypeptide; and/or a third nucleic acid sequence encoding a chimeric human/non-human MHC II β polypeptide, wherein a human portion of the chimeric MHC II β polypeptide comprises an extracellular portion (or part thereof, e.g., one or more extracellular domains) of a human MHC II β polypeptide; wherein the non-human animal expresses functional chimeric human/non-human MHC I and MHC II proteins from its endogenous non-human MHC locus. In one embodiment, the first, second, and/or third nucleic acid sequences are respectively located the endogenous non-human MHC I, MHC II α and MHC II β loci. In one embodiment, wherein the non-human animal is a mouse, the first, second, and/or third nucleic acid sequences are located at the endogenous mouse MHC locus on mouse chromosome 17. In one embodiment, the first nucleic acid sequence is located at the endogenous non-human MHC I locus. In one embodiment, the second nucleic acid sequence is located at the endogenous non-human MHC II α locus. In one embodiment, the third nucleic acid sequence is located at the endogenous non-human MHC II β locus.

In one embodiment, the non-human animal only expresses the chimeric human/non-human MHC I, MHC II α and/or MHC β II polypeptides and does not express endogenous non-human MHC polypeptides (e.g., functional endogenous MHC I, II α and/or II β polypeptides) from the endogenous non-human MHC locus. In one embodiment, the animal described herein expresses a functional chimeric MHC I and a functional chimeric MHC II on the surface of its cells, e.g., antigen presenting cells, etc. In one embodiment, the only MHC I and MHC II expressed by the animal on a cell surface are chimeric MHC I and chimeric MHC II, and the animal does not express any endogenous MHC I and MHC II on a cell surface.

In one embodiment, the chimeric human/non-human MHC I polypeptide comprises in its human portion a peptide binding cleft, e.g., of a human MHC I polypeptide. In one aspect, the human portion of the chimeric polypeptide comprises an extracellular portion of a human MHC I. In this embodiment, the human portion of the chimeric polypeptide comprises an extracellular domain of an α chain of a human MHC I. In one embodiment, the human portion of the chimeric polypeptide comprises α1 and α2 domains of a human MHC I. In another embodiment, the human portion of the chimeric polypeptide comprises α1, α2, and α3 domains of a human MHC I.

In one aspect, a human portion of the chimeric MHC II α polypeptide and/or a human portion of the chimeric MHC II β polypeptide comprises a peptide-binding domain of a human MHC II α polypeptide and/or human MHC II β polypeptide, respectively. In one aspect, a human portion of the chimeric MHC II α and/or β polypeptide comprises an extracellular portion of a human MHC II α and/or β polypeptide, respectively. In one embodiment, a human portion of the chimeric MHC II α polypeptide comprises α1 domain of a human MHC II α polypeptide; in another embodiment, a human portion of the chimeric MHC II α polypeptide comprises α1 and α2 domains of a human MHC II α polypeptide. In an additional embodiment, a human portion of the chimeric MHC II β polypeptide comprises β1 domain of a human MHC II β polypeptide; in another embodiment, a human portion of the chimeric MHC II β polypeptide comprises β1 and β2 domains of a human MHC II β polypeptide.

In some embodiments, the human or humanized MHC I polypeptide may be derived from a functional human HLA molecule encoded by any of HLA-A, HLA-B, HLA-C, HLA-E, HLA-F, or HLA-G loci. The human or humanized MHC II polypeptide may be derived from a functional human HLA molecule encoded by an of HLA-DP, -DQ, and -DR loci. A list of commonly used HLA antigens and alleles is described in Shankarkumar et al. ((2004) The Human Leukocyte Antigen (HLA) System, Int. J. Hum. Genet. 4(2):91-103), incorporated herein by reference. Shankarkumar et al. also present a brief explanation of HLA nomenclature used in the art. Additional information regarding HLA nomenclature and various HLA alleles can be found in Holdsworth et al. (2009) The HLA dictionary 2008: a summary of HLA-A, —B, —C, -DRB1/3/4/5, and DQB1 alleles and their association with serologically defined HLA-A, —B, —C, -DR, and -DQ antigens, Tissue Antigens 73:95-170, and a recent update by Marsh et al. (2010) Nomenclature for factors of the HLA system, 2010, Tissue Antigens 75:291-455, both incorporated herein by reference. In some embodiments, the MHC I or MHC II polypeptides may be derived from any functional human HLA-A, B, C, DR, or DQ molecules. Thus, the human or humanized MHC I and/or II polypeptides may be derived from any functional human HLA molecules described therein. In some embodiments, all MHC I and MHC II polypeptides expressed on a cell surface comprise a portion derived from human HLA molecules.

Of particular interest are human HLA molecules, specific polymorphic HLA alleles, known to be associated with a number of human diseases, e.g., human autoimmune diseases. In fact, specific polymorphisms in HLA loci have been identified that correlate with development of rheumatoid arthritis, type I diabetes, Hashimoto's thyroiditis, multiple sclerosis, myasthenia gravis, Graves' disease, systemic lupus erythematosus, celiac disease, Crohn's disease, ulcerative colitis, and other autoimmune disorders. See, e.g., Wong and Wen (2004) What can the HLA transgenic mouse tell us about autoimmune diabetes?, Diabetologia 47:1476-87; Tanej a and David (1998) HLA Transgenic Mice as Humanized Mouse Models of Disease and Immunity, J. Clin. Invest. 101:921-26; Bakker et al. (2006), A high-resolution HLA and SNP haplotype map for disease association studies in the extended human MHC, Nature Genetics 38:1166-72 and Supplementary Information; and International MHC and Autoimmunity Genetics Network (2009) Mapping of multiple susceptibility variants within the MHC region for 7 immune-mediated diseases, Proc. Natl. Acad. Sci. USA 106:18680-85. Thus, the human or humanized MHC I and/or II polypeptides may be derived from a human HLA molecule known to be associated with a particular disease, e.g., autoimmune disease.

In one specific aspect, the human or humanized MHC I polypeptide is derived from human HLA-A. In a specific embodiment, the HLA-A polypeptide is an HLA-A2 polypeptide (e.g., and HLA-A2.1 polypeptide). In one embodiment, the HLA-A polypeptide is a polypeptide encoded by an HLA-A*0201 allele, e.g., HLA-A*02:01:01:01 allele. The HLA-A*0201 allele is commonly used amongst the North American population. Although the present Examples provide U.S. Pat. Nos. 9,615,550 and 10,154,658 to describe this particular HLA sequence, any suitable HLA-A sequence is encompassed herein, e.g., polymorphic variants of HLA-A2 exhibited in human population, sequences with one or more conservative or non-conservative amino acid modifications, nucleic acid sequences differing from the sequence described herein due to the degeneracy of genetic code, etc.

In another specific aspect, the human portion of the chimeric MHC I polypeptide is derived from human MHC I selected from HLA-B and HLA-C. In one aspect, it is derived from HLA-B, e.g., HLA-B27. In another aspect, it is derived from HLA-A3, -B7, -Cw6, etc.

In one specific aspect, the human portions of the humanized MHC II α and β polypeptides described herein are derived from human HLA-DR, e.g., HLA-DR2. Typically, HLA-DR α chains are monomorphic, e.g., the α chain of HLA-DR complex is encoded by HLA-DRA gene (e.g., HLA-DRα*01 gene). On the other hand, the HLA-DR β chain is polymorphic. Thus, HLA-DR2 comprises an α chain encoded by HLA-DRA gene and a β chain encoded by HLA-DR1β*1501 gene. Although the present Examples cite U.S. Pat. Nos. 8,847,005 and 9,043,996 to describe these particular HLA sequences; any suitable HLA-DR sequences are encompassed herein, e.g., polymorphic variants exhibited in human population, sequences with one or more conservative or non-conservative amino acid modifications, nucleic acid sequences differing from the sequences described herein due to the degeneracy of genetic code, etc.

The human portions of the chimeric MHC II α and/or β polypeptide may be encoded by nucleic acid sequences of HLA alleles known to be associated with common human diseases. Such HLA alleles include, but are not limited to, HLA-DRB1*0401, -DRB1*0301, -DQA1*0501, -DQB1*0201, DRB1*1501, -DRB1*1502, -DQB1*0602, -DQA1*0102, -DQA1*0201, -DQB1*0202, -DQA1*0501, and combinations thereof. For a summary of HLA allele/disease associations, see Bakker et al. (2006), supra, incorporated herein by reference.

In one aspect, the non-human portion of a chimeric human/non-human MHC I, MHC II α and/or MHC II β polypeptide(s) comprises transmembrane and/or cytoplasmic domains of an endogenous non-human (e.g., rodent, e.g., mouse, rat, etc.) MHC I, MHC II α and/or MHC II β polypeptide(s), respectively. Thus, the non-human portion of the chimeric human/non-human MHC I polypeptide may comprise transmembrane and/or cytoplasmic domains of an endogenous non-human MHC I polypeptide. The non-human portion of a chimeric MHC II α polypeptide may comprise transmembrane and/or cytoplasmic domains of an endogenous non-human MHC II α polypeptide. The non-human portion of a chimeric human/non-human MHC II β polypeptide may comprise transmembrane and/or cytoplasmic domains of an endogenous non-human MHC II β polypeptide. In one aspect, the non-human animal is mouse, and a non-human portion of the chimeric MHC I polypeptide is derived from a mouse H-2K protein. In one aspect, the animal is a mouse, and non-human portions of the chimeric MHC II α and β polypeptides are derived from a mouse H-2E protein. Thus, a non-human portion of the chimeric MHC I polypeptide may comprise transmembrane and cytoplasmic domains derived from a mouse H-2K, and non-human portions of the chimeric MHC II α and β polypeptides may comprise transmembrane and cytoplasmic domains derived from a mouse H-2E protein. Although specific H-2K and H-2E sequences are contemplated in U.S. Pat. Nos. 9,615,550 and 10,154,658, cited in the Examples, any suitable sequences, e.g., polymorphic variants, conservative/non-conservative amino acid substitutions, etc., are encompassed herein. In one aspect, the non-human animal is a mouse, and the mouse does not express functional endogenous MHC polypeptides from its H-2D locus. In some embodiments, the mouse is engineered to lack all or a portion of an endogenous H-2D locus. In other aspects, the mouse does not express any functional endogenous mouse MHC I and MHC II on a cell surface.

A chimeric human/non-human polypeptide may be such that it comprises a human or a non-human leader (signal) sequence. In one embodiment, the chimeric MHC I polypeptide comprises a non-human leader sequence of an endogenous MHC I polypeptide. In one embodiment, the chimeric MHC II α polypeptide comprises a non-human leader sequence of an endogenous MHC II α polypeptide. In one embodiment, the chimeric MHC II β polypeptide comprises a non-human leader sequence of an endogenous MHC II β polypeptide. In an alternative embodiment, the chimeric MHC I, MHC II α and/or MHC II β polypeptide(s) comprises a non-human leader sequence of MHC I, MHC II α and/or MHC II β polypeptide(s), respectively, from another non-human animal, e.g., another rodent or another mouse strain. Thus, the nucleic acid sequence encoding the chimeric MHC I, MHC II α and/or MHC II β polypeptide may be operably linked to a nucleic acid sequence encoding a non-human MHC I, MHC II α and/or MHC II β leader sequence, respectively. In yet another embodiment, the chimeric MHC I, MHC II α and/or MHC II β polypeptide(s) comprises a human leader sequence of human MHC I, human MHC II α and/or human MHC II β polypeptide, respectively (e.g., a leader sequence of human HLA-A2, human HLA-DRα and/or human HLA-DRβ1*1501, respectively).

A chimeric human/non-human MHC I, MHC II α and/or MHC II β polypeptide may comprise in its human portion a complete or substantially complete extracellular domain of a human MHC I, human MHC II α and/or human MHC II β polypeptide, respectively. Thus, a human portion may comprise at least 80%, preferably at least 85%, more preferably at least 90%, e.g., 95% or more of the amino acids encoding an extracellular domain of a human MHC I, human MHC II α and/or human MHC II βpolypeptide (e.g., human HLA-A2, human HLA-DRα and/or human HLA-DRβ1*1501). In one example, substantially complete extracellular domain of the human MHC I, human MHC II α and/or human MHC II β polypeptide lacks a human leader sequence. In another example, the chimeric human/non-human MHC I, chimeric human/non-human MHC II α and/or the chimeric human/non-human MHC II β polypeptide comprises a human leader sequence.

Moreover, the chimeric MHC I, MHC II α and/or MHC II β polypeptide may be operably linked to (e.g., be expressed under the regulatory control of) endogenous non-human promoter and regulatory elements, e.g., mouse MHC I, MHC II α and/or MHC II βregulatory elements, respectively. Such arrangement will facilitate proper expression of the chimeric MHC I and/or MHC II polypeptides in the non-human animal, e.g., during immune response in the non-human animal.

In a further embodiment, a non-human animal of the invention, e.g., a rodent, e.g., a mouse, comprises (e.g., at an endogenous β2 microglobulin locus) a nucleic acid sequence encoding a human or humanized β2 microglobulin. β2 microglobulin or the light chain of the MHC class I complex (also abbreviated “β2M”) is a small (12 kDa) non-glycosylated protein, that functions primarily to stabilize the MHC I α chain. Generation of human or humanized β2 microglobulin animals is described in detail in U.S. Pat. No. 9,615,550 and is incorporated herein by reference.

The nucleotide sequence encoding the human or humanized β2 microglobulin polypeptide may comprise nucleic acid residues corresponding to the entire human β2 microglobulin gene. Alternatively, the nucleotide sequence may comprise nucleic acid residues encoding amino acid sequence set forth in amino acids 21-119 of a human β2 microglobulin protein (i.e., amino acid residues corresponding to the mature human β2 microglobulin). In an alternative embodiment, the nucleotide sequence may comprise nucleic acid residues encoding amino acid sequence set forth in amino acids 23-115 of a human β2 microglobulin protein, for example, amino acid sequence set forth in amino acids 23-119 of a human β2 microglobulin protein. The nucleic and amino acid sequences of human β2 microglobulin are described in Gussow et al., supra, incorporated herein by reference.

Thus, the human or humanized β2 microglobulin polypeptide may comprise amino acid sequence set forth in amino acids 23-115 of a human β2 microglobulin polypeptide, e.g., amino acid sequence set forth in amino acids 23-119 of a human β2 microglobulin polypeptide, e.g., amino acid sequence set forth in amino acids 21-119 of a human β2 microglobulin polypeptide. Alternatively, the human β2 microglobulin may comprise amino acids 1-119 of a human β2 microglobulin polypeptide.

In some embodiments, the nucleotide sequence encoding a human or humanized β2 microglobulin comprises a nucleotide sequence set forth in exon 2 to exon 4 of a human β2 microglobulin gene. Alternatively, the nucleotide sequence comprises nucleotide sequences set forth in exons 2, 3, and 4 of a human β2 microglobulin gene. In this embodiment, the nucleotide sequences set forth in exons 2, 3, and 4 are operably linked to allow for normal transcription and translation of the gene. Thus, in one embodiment, the human sequence comprises a nucleotide sequence corresponding to exon 2 to exon 4 of a human β2 microglobulin gene. In a specific embodiment, the human sequence comprises a nucleotide sequence corresponding to exon 2 to about 267 bp after exon 4 of a human β2 microglobulin gene. In a specific embodiment, the human sequence comprises about 2.8 kb of a human β2 microglobulin gene.

Thus, the human or humanized β2 microglobulin polypeptide may be encoded by a nucleotide sequence comprising nucleotide sequence set forth in exon 2 to exon 4 of a human β2 microglobulin, e.g., nucleotide sequence corresponding to exon 2 to exon 4 of a human β2 microglobulin gene. Alternatively, the polypeptide may be encoded by a nucleotide sequence comprising nucleotide sequences set forth in exons 2, 3, and 4 of a human β2 microglobulin gene. In a specific embodiment, the human or humanized β2 microglobulin polypeptide is encoded by a nucleotide sequence corresponding to exon 2 to about 267 bp after exon 4 of a human β2 microglobulin gene. In another specific embodiment, the human or humanized polypeptide is encoded by a nucleotide sequence comprising about 2.8 kb of a human β2 microglobulin gene. As exon 4 of the β2 microglobulin gene contains the 5′ untranslated region, the human or humanized polypeptide may be encoded by a nucleotide sequence comprising exons 2 and 3 of the β2 microglobulin gene.

It would be understood by those of ordinary skill in the art that although specific nucleic acid and amino acid sequences to generate genetically engineered animals are described herein, sequences of one or more conservative or non-conservative amino acid substitutions, or sequences differing from those described herein due to the degeneracy of the genetic code, are also provided.

Therefore, a non-human animal that expresses a human β2 microglobulin sequence is provided, wherein the β2 microglobulin sequence is at least about 85%, 90%, 95%, 96%, 97%, 98%, or 99% identical to a human β2 microglobulin sequence. In a specific embodiment, the β2 microglobulin sequence is at least about 90%, 95%, 96%, 97%, 98%, or 99% identical to the human β2 microglobulin sequence described herein. In one embodiment, the human β2 microglobulin sequence comprises one or more conservative substitutions. In one embodiment, the human β2 microglobulin sequence comprises one or more non-conservative substitutions.

In addition, provided are non-human animals wherein the nucleotide sequence encoding a human or humanized β2 microglobulin protein also comprises a nucleotide sequence set forth in exon 1 of a non-human β2 microglobulin gene. Thus, in a specific embodiment, the non-human animal comprises in its genome a nucleotide sequence encoding a human or humanized β2 microglobulin wherein the nucleotide sequence comprises exon 1 of a non-human β2 microglobulin and exons 2, 3, and 4 of a human β2 microglobulin gene. Thus, the human or humanized β2 microglobulin polypeptide is encoded by exon 1 of a non-human β2 microglobulin gene and exons 2, 3, and 4 of a human β2 microglobulin gene (e.g., exons 2 and 3 of a human β2 microglobulin gene).

In one embodiment, the non-human animal (e.g., rodent, e.g., mouse) of the invention, in addition to a nucleotide sequence encoding a chimeric CD8 protein, further comprises a nucleic acid sequence encoding a human or humanized MHC I protein, such that the chimeric CD8 protein expressed on the surface of a T cell of the animal is capable of associating, binding and/or interacting with a human or humanized MHC I expressed on a surface of a second cell, e.g., an antigen presenting cell. In one embodiment, the MHC I protein comprises an extracellular domain of a human MHC I polypeptide. In one embodiment, the animal further comprises a human or humanized β2 microglobulin polypeptide. Exemplary genetically modified animals expressing a human or humanized MHC I polypeptide and/or β2 microglobulin polypeptide are described in U.S. Pat. Nos. 9,615,550 and 9,591,835, both incorporated herein by reference in their entireties. Thus, in one embodiment, the animal comprising chimeric CD8 protein described herein may further comprise a humanized MHC I complex, wherein the humanized MHC I complex comprises: (1) a humanized MHC I polypeptide, e.g., wherein the humanized MHC I polypeptide comprises a human MHC I extracellular domain and transmembrane and cytoplasmic domains of an endogenous (e.g., mouse) MHC I, e.g., wherein the humanized MHC I comprises α1, α2, and α3 domains of a human MHC I polypeptide, and (2) a human or humanized β2 microglobulin polypeptide (e.g., the animal comprises in its genome a nucleotide sequence set forth in exons 2, 3, and 4 of a human β2 microglobulin). In one aspect, both humanized MHC I and human or humanized β2 microglobulin polypeptides are encoded by nucleotide sequences located at endogenous MHC I and β2 microglobulin loci, respectively; in one aspect, the animal does not express functional endogenous MHC I and β2 microglobulin polypeptides. Thus, the MHC I expressed by the animals may be a chimeric human/non-human, e.g., human/rodent (e.g., human/mouse) MHC I polypeptide. A human portion of the chimeric MHC I polypeptide may be derived from a human HLA class I protein selected from the group consisting of HLA-A, HLA-B, and HLA-C, e.g., HLA-A2, HLA-B27, HLA-B7, HLA-Cw6, or any other HLA class I molecule present in a human population. In the embodiment, wherein the animal is a mouse, a non-human (i.e., a mouse) portion of the chimeric MHC I polypeptide may be derived from a mouse MHC I protein selected from H-2D, H-2K and H-2L.

In one embodiment, the non-human animal (e.g., rodent, e.g., mouse) of the invention further comprises a nucleotide sequence encoding a human or humanized MHC II protein, such that the chimeric CD4 protein expressed on the surface of a T cell of the animal is capable of interacting with a human or humanized MHC II expressed on a surface of a second cell, e.g., an antigen presenting cell. In one embodiment, the MHC II protein comprises an extracellular domain of a human MHC II α polypeptide and an extracellular domain of a human MHC II β polypeptide. Exemplary genetically modified animals expressing a human or humanized MHC II polypeptide are described in U.S. Pat. No. 8,847,005, issued Sep. 30, 2014, and U.S. Pat. No. 9,043,996, incorporated herein by reference in their entireties. Thus, in one embodiment, the animal comprising chimeric CD4 protein described herein may further comprise a humanized MHC II protein, wherein the humanized MHC II protein comprises: (1) a humanized MHC II α polypeptide comprising a human MHC II α extracellular domain and transmembrane and cytoplasmic domains of an endogenous, e.g., mouse, MHC II, wherein the human MHC II α extracellular domain comprises α1 and α2 domains of a human MHC II α and (2) a humanized MHC II β polypeptide comprising a human MHC II β extracellular domain and transmembrane and cytoplasmic domains of an endogenous, e.g., mouse, MHC II, wherein the human MHC II β extracellular domain comprises β1 and β2 domains of a human MHC II β. In one aspect, both humanized MHC II α and β polypeptides are encoded by nucleic acid sequences located at endogenous MHC II α and β loci, respectively; in one aspect, the animal does not express functional endogenous MHC II α and β polypeptides. Thus, the MHC II expressed by the animals may be a chimeric human/non-human, e.g., human/rodent (e.g., human/mouse) MHC II protein. A human portion of the chimeric MHC II protein may be derived from a human HLA class II protein selected from the group consisting of HLA-DR, HLA-DQ, and HLA-DP, e.g., HLA-DR4, HLA-DR2, HLA-DQ2.5, HLA-DQ8, or any other HLA class II molecule present in a human population. In the embodiment, wherein the animal is a mouse, a non-human (i.e., a mouse) portion of the chimeric MHC II polypeptide may be derived from a mouse MHC II protein selected from H-2E and H-2A.

Various other embodiments of a genetically modified non-human animal, e.g. rodent, e.g., rat or mouse, would be evident to one skilled in the art from the present disclosure and from the disclosure of U.S. Pat. Nos. 8,847,005; 9,043,996; 9,591,835; 9,615,550; and U.S. Pat. No. 10,154,658; each of which is incorporated herein by reference.

In various embodiments, the genetically modified non-human animals described herein make cells, e.g., APCs, with human or humanized MHC I and II on the cell surface and, as a result, present peptides as epitopes for T cells in a human-like manner, because substantially all of the components of the complex are human or humanized. The genetically modified non-human animals of the invention can be used to study the function of a human immune system in the humanized animal; for identification of antigens and antigen epitopes that elicit immune response (e.g., T cell epitopes, e.g., unique human cancer epitopes), e.g., for use in vaccine development; for evaluation of vaccine candidates and other vaccine strategies; for studying human autoimmunity; for studying human infectious diseases; and otherwise for devising better therapeutic strategies based on human MHC expression.

In some embodiments, a mouse as described herein comprises:
(I) germ cells and CD3⁻ somatic cells that comprise:
   (A) at the endogenous TCR γ locus:
      a replacement of all endogenous TCR Vγ segments with a full repertoire of unrearranged human TCR Vγ segments,
      a replacement of all endogenous TCR Jγ segments with a full repertoire of unrearranged human TCR Jγ segments, and
      a replacement of all TCR γ constant region gene sequences with a full repertoire of human TCR γ constant region gene sequences; and
(B) at the endogenous TCR δ locus:
   a replacement of all endogenous TCR Vδ segments with a full repertoire of unrearranged human TCR Vδ segments,
      a replacement of all endogenous TCR Dδ segments with a full repertoire of unrearranged human TCR Dδ segments,
      a replacement of all endogenous TCR Jδ segments with a full repertoire of unrearranged human TCR Jδ segments, and
      a replacement of an endogenous TCR δ constant region gene sequence with a human TCR δ constant region gene sequence; and
   (C) at the endogenous TCR α locus:
      a replacement of all endogenous TCR V α segments with a full repertoire of unrearranged human TCR Vα segments, and
      a replacement of all endogenous TCR Jα segments with a full repertoire of unrearranged human TCR Jα segments, and
   (D) at the endogenous TCR β locus:
      a replacement of all endogenous TCR Vβ segments with a full repertoire of unrearranged human TCR Vβ segments,
      a replacement of all endogenous TCR Dβ segments with a full repertoire of unrearranged human TCR Dβ segments,
      a replacement of all endogenous TCR Jβ segments with a full repertoire of unrearranged human TCR Jβ segments,
   (E) a first nucleotide sequence encoding a chimeric human/mouse CD4 co-receptor that comprises D1, D2 and D3 domains of a human CD4 polypeptide operably linked to D4, transmembrane and cytoplasmic domains of a mouse CD4 polypeptide;
   (F) a second nucleotide sequence encoding a chimeric human/mouse CD8α polypeptide and a third nucleotide sequence encoding a chimeric human/mouse CD8β polypeptide,
      wherein the chimeric human/mouse CD8α polypeptide comprises an IgV-like domain of a human CD8α polypeptide operably linked to transmembrane and cytoplasmic domains of a mouse CD8α polypeptide and wherein the chimeric human/mouse CD8β polypeptide comprises an IgV-like domain of a human CD8β polypeptide operably linked to transmembrane and cytoplasmic domains of a mouse CD8β polypeptide;
   (G) a first nucleic acid sequence encoding a chimeric human/mouse MHC II α polypeptide and a second nucleic acid sequence encoding a chimeric human/mouse MHC II β polypeptide,
      wherein the chimeric human/mouse MHC II α polypeptide comprises α1 and α2 domains of a human HLA class II α polypeptide operably linked to transmembrane and cytoplasmic domains of a mouse MHC II α polypeptide and wherein the chimeric human/mouse MHC II β polypeptide comprises β1 and β2 domains of a human HLA class II β polypeptide operably linked to transmembrane and cytoplasmic domains of a mouse MHC II β polypeptide;
   (H) a third nucleic acid sequence encoding a chimeric human/mouse MHC I polypeptide comprising α1, α2, and α3 domains of a human HLA class I polypeptide operably linked to transmembrane and cytoplasmic domains of a mouse MHC class I polypeptide; and
   (I) a polynucleotide encoding a human or humanized β2 microglobulin polypeptide and comprising a nucleotide sequence comprising the nucleotide sequence set forth in exon 1 of a mouse β2 microglobulin gene operably linked to the nucleotide sequence set forth in exon 2, exon 3, and exon 4 of a human β2 microglobulin gene,
(II) wherein the mouse further comprises a CD3⁺ T cell that expresses, on its surface, a functional human TCR comprising a human TCR γ polypeptide and a human TCR δ polypeptide and a CD3⁺ T cell that expresses, on its surface, a functional human or humanized TCR comprising a human or humanized TCR α polypeptide and a human or humanized TCR (3 polypeptide, and optionally wherein the mouse expresses:
   (e) the chimeric human/mouse CD4 co-receptor,
   (f) a chimeric CD8 co-receptor comprising the chimeric human/mouse CD8α polypeptide and the chimeric human/mouse CD8β polypeptide,
   (h) a chimeric MHC II complex comprising the chimeric human/mouse MHC II α polypeptide and the chimeric human/mouse MHC II β polypeptide, wherein the chimeric MHC II complex is capable of binding the chimeric human/mouse CD4 co-receptor,
   (i) the chimeric human/mouse MHC I polypeptide, wherein the chimeric MHC I polypeptide is capable of binding the chimeric CD8 co-receptor, and
   (j) the human or humanized β2 microglobulin polypeptide.

In some embodiments, a non-human animal as described herein comprises two copies of one or more of the modified loci described herein. In some embodiments, a non-human animal as described herein comprises two copies of an unrearranged human or humanized TCRγ locus, two copies of the unrearranged human or humanized TCRδ locus, two copies the unrearranged human or humanized TCRα locus, two copies of the unrearranged human or humanized TCRβ locus, two copies of the human or humanized CD4 locus, two copies of the human or humanized CD8α locus, two copies of the human or humanized CD8β locus, two copies of the human or humanized MHC I locus, and/or two copies of the human or humanized MHC II α and/or MHC II β loci. Thus, the non-human animal is homozygous for one or more unrearranged human or humanized TCRγ, TCRδ, TCRα, and/or TCRβ loci, one or more human or humanized co-receptor loci, and/or one or more MHC loci. In some embodiments of the invention, the non-human animal comprises one copy of the unrearranged human or humanized TCRγ locus, one copy of the unrearranged human or humanized TCRδ variable gene locus, one copy of the unrearranged human or humanized TCRα variable gene locus, one copy of the unrearranged human or humanized TCRβ variable gene locus, one copy of the human or humanized CD4 locus, one copy of the human or humanized CD8α locus, one copy of the human or humanized CD8β locus, one copy of the human or humanized MHC I locus, and/or one copy of the human or humanized MHC II α and/or MHC II β loci. Thus, the non-human animal may be heterozygous for unrearranged human or humanized TCRγ, TCRδ, TCRα, and/or TCRβ loci, one or more human or humanized co-receptor loci, and/or one or more human or humanized MHC loci. In some embodiments, a non-human animal (e.g., a mouse) is heterozygous or homozygous for an unrearranged human TCRγ locus, e.g., wherein the non-human animal comprises an unrearranged human TCR Vγ segment and an unrearranged human Jγ segment operably linked to a human TCR Cγ gene. In some embodiments, a non-human animal (e.g., a mouse) is heterozygous or homozygous for an unrearranged human TCRδ locus, e.g., wherein the non-human animal comprises an unrearranged human TCR Vδ segment, an unrearranged human TCR Dδ segment, and an unrearranged human Jδ segment operably linked to a human TCR Cδ gene.

In some embodiments, a non-human animal as described herein that is heterozygous or homozygous for human TRD and TRG loci, and humanized TRA, TRB, MHC I, MHC II, CD4, CD8, and β2 microglobulin loci, comprises a population of CD45+CD3+ T cells in its spleen, thymus, mesenteric lymph nodes (MLN), skin, gut mucosa, and/or among intraepithelial lymphocytes (IEL) isolated from its colon or small intestine, wherein a percentage of the population of CD45+CD3+ T cells expresses a human γ/δ TCR. In some embodiments, the percentage of splenic, thymic, MLN, and/or IEL CD45+CD3+ T cells that express human γ/δ TCR in a γ and/or δ TCR mouse embodiment as described herein is comparable (e.g., not significantly different, wherein any difference is not statistically significant, within 10 percentage points of each other, etc.) to the percentage of CD45+CD3+ T cells that express murine γ/δ TCR in a wildtype mouse. In some embodiments, the percentage of splenic, thymic, MLN, and/or IEL CD45+CD3+ T cells that express human γ/δ TCR in a γ and/or δ TCR mouse embodiment as described herein is greater than (e.g., 1.5-fold to 3-fold) the percentage of CD45+CD3+ T cells that express murine γ/δ TCR in a wildtype mouse.

The genetically modified non-human animal of the invention may be selected from a group consisting of a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable ES cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo.

In one aspect, the non-human animal is a mammal. In one aspect, the non-human animal is a small mammal, e.g., of the superfamily Dipodoidea or Muroidea. In one embodiment, the genetically modified animal is a rodent. In one embodiment, the rodent is selected from a mouse, a rat, and a hamster. In one embodiment, the rodent is selected from the superfamily Muroidea. In one embodiment, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, white-tailed rats, Malagasy rats and mice), Platacanthomyidae (e.g., spiny dormice), and Spalacidae (e.g., mole rates, bamboo rats, and zokors). In a specific embodiment, the genetically modified rodent is selected from a true mouse or rat (family Muridae), a gerbil, a spiny mouse, and a crested rat. In one embodiment, the genetically modified mouse is from a member of the family Muridae. In one embodiment, the animal is a rodent. In a specific embodiment, the rodent is selected from a mouse and a rat. In one embodiment, the non-human animal is a mouse.

In a specific embodiment, the non-human animal is a rodent that is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In another embodiment, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2 (see, e.g., Festing et al. (1999) Revised nomenclature for strain 129 mice, Mammalian Genome 10:836, see also, Auerbach et al (2000) Establishment and Chimera Analysis of 129/SvEv- and C57BL/6-Derived Mouse Embryonic Stem Cell Lines). In an embodiment, the genetically modified mouse is a mix of an aforementioned 129 strain and an aforementioned C57BL/6 strain. In another specific embodiment, the mouse is a mix of aforementioned 129 strains, or a mix of aforementioned BL/6 strains. In a specific embodiment, the 129 strain of the mix is a 129S6 (129/SvEvTac) strain. In another embodiment, the mouse is a BALB strain, e.g., BALB/c strain. In yet another embodiment, the mouse is a mix of a BALB strain and another aforementioned strain. Non-human animals as provided herein may be a mouse derived from any combination of the aforementioned strains.

In one embodiment, the non-human animal is a rat. In one embodiment, the rat is selected from a Wistar rat, an LEA strain, a Sprague Dawley strain, a Fischer strain, F344, F6, and Dark Agouti. In one embodiment, the rat strain is a mix of two or more strains selected from the group consisting of Wistar, LEA, Sprague Dawley, Fischer, F344, F6, and Dark Agouti.

In some aspects of the invention, the non-human animal comprising an unrearranged human or humanized TCR γ, TCR δ, TCRα, and/or TCRβ gene locus (comprising unrearranged human TCR γ, TCR δ, TCRα, and/or TCRβ variable V, (D), and J segments, respectively) retains an endogenous non-human TCR γ, TCR δ, TCRα, and/or TCRβ gene segment or gene locus. In one embodiment, a retained endogenous non-human TCR γ, TCR δ, TCRα, and/or TCRβ segment is a functional gene segment and is capable of rearranging (or rearranges), e.g., in a T cell, with a human TCR γ, TCR δ, TCRα, and/or TCRβ segment, respectively, found at the same locus according to the 12/23 rule of recombination. See, Olaru A., supra. In one embodiment, the endogenous non-human TCR γ, TCR δ, TCRα, and/or TCRβ is a non-functional gene segment or gene locus. In one embodiment, the non-functional locus is an inactivated locus, e.g., an inverted locus (e.g., the coding nucleic acid sequence of the variable gene locus is in inverted orientation with respect to the constant region gene sequence, such that no successful rearrangements are possible utilizing variable region segments from the inverted locus). In one embodiment, the humanized TCR γ, TCR δ, TCRα, and/or TCRβ gene segment or variable gene locus is positioned between the endogenous non-human TCR γ, TCR δ, TCRα, and/or TCRβ variable gene locus and the endogenous non-human TCR γ, TCR δ, TCRα, and/or TCRβ constant region gene locus, respectively. In one embodiment, the humanized TCR γ, TCR δ, TCRα, and/or TCRβ gene segment or variable gene locus is positioned between the endogenous non-human TCR γ, TCR δ, TCRα, and/or TCRβ variable gene locus, respectively, and a human TCR γ, TCR δ, TCRα, and/or TCRβ constant region gene locus, respectively.

In various embodiments of the invention, the unrearranged human or humanized TCR variable gene locus (e.g., TCRα TCRβ, TCR γ and/or TCRδ variable gene locus) is comprised in the germline of the non-human animal (e.g., rodent, e.g., mouse or rat), e.g., the non-human animal comprises germ cells (sperm and oocytes) that comprise the modified loci as described herein. In various embodiments, the replacements of TCR V(D)J segments by unrearranged human TCR V(D)J segments (e.g., Vα and Jα; Vβ and Dβ and Jβ; Vδ and Dδ and Jδ; Vγ and Jγ segments) are at an endogenous non-human TCR variable locus (or loci), wherein the unrearranged human V and J and/or V and D and J segments are operably linked to human or non-human TCR constant region gene sequences.

In one aspect, the non-human animal (e.g., rodent, e.g., mouse or rat) comprising human or humanized TCRγ and/or TCRδ loci, and optionally TCRα and/or TCR β loci described herein expresses a humanized T cell receptor comprising a human variable region and a non-human (e.g., rodent, e.g., mouse or rat) constant domain on a surface of a T cell. In some aspects, the non-human animal is capable or expressing a diverse repertoire of humanized T cell receptors that recognize a variety of presented antigens.

In addition to a genetically engineered non-human animal, a non-human embryonic stem (ES) cell line or germ cell line is also provided, as well as an embryo (e.g., a rodent, e.g., a mouse or a rat embryo) embryo comprising and/or derived from the ES cell. An ES cell, germ cell, and/or embryo as described herein comprises a genetically modified locus as described herein, e.g., a human or humanized TCRG and/or TCRD locus, and optionally a human or humanized TCRA, TCRB, CD4, CD8α, CD80, MHC I and/or MHC IIα and/or MHC IIβ loci.

Also provided is a tissue, wherein the tissue is derived from a non-human animal (e.g., a rodent, e.g., a mouse or a rat) as described herein, and comprises a cell that expresses human TCR γ and/or TCR δ proteins from human or humanized TCRG and/or TCRD loci, respectively.

In some embodiments, a method for making a human TCRγ and/or a human TCR protein is provided, comprising expressing in a single cell a human TCRγ and/or a human TCR protein from a nucleotide construct as described herein. In one embodiment, the nucleotide construct is a viral vector; in a specific embodiment, the viral vector is a lentiviral vector. In one embodiment, the cell is selected from a CHO, COS, 293, HeLa, and a retinal cell expressing a viral nucleic acid sequence (e.g., a PERC.6™ cell).

In one aspect, a cell that expresses a human TCRγ and/or a human TCR protein is provided. In one embodiment, the cell comprises an expression vector comprising a human TCRγ and/or a human TCR protein as described herein. In one embodiment, the cell is selected from CHO, COS, 293, HeLa, and a retinal cell expressing a viral nucleic acid sequence (e.g., a PERC.6™ cell).

A human TCRγ and/or a human TCR protein made by a non-human animal as described herein is also provided. Thus, the human TCR protein comprises human complementary determining regions (i.e., human CDR1, 2, and 3) in its variable domain and a human constant region. Also provided are nucleic acids that encode the human TCR variable domains generated by a non-human animal described herein.

In addition, a non-human cell isolated from a non-human animal as described herein is provided. In one embodiment, the cell is an ES cell. In one embodiment, the cell is a T cell, e.g., a γ/δ T cell. Also provided is a non-human cell that expresses a TCR protein comprising human TCR γ protein and/or human TCR δ protein.

Also provided is a non-human cell comprising a chromosome or fragment thereof of a non-human animal as described herein. In one embodiment, the non-human cell comprises a nucleus of a non-human animal as described herein. In one embodiment, the non-human cell comprises the chromosome or fragment thereof as the result of a nuclear transfer.

In one aspect, a hybridoma or quadroma is provided, derived from a cell of a non-human animal as described herein. In one embodiment, the non-human animal is a mouse or rat.

Making Genetically Modified Non-Human Animals that Mount Substantially Humanized T Cell Immune Responses

Provided is a method for making a genetically engineered non-human animal (e.g., a genetically engineered rodent, e.g., a mouse or rat) described herein. Generally, the methods comprise inserting into the genome of the non-human animal an unrearranged T cell receptor (TCR) γ variable gene locus comprising at least one human Vγ segment and at least one human Jγ segment, operably linked to a human or non-human TCRγ constant region gene sequence and/or an unrearranged TCR δ variable gene locus comprising at least one human Vδ segment, at least one human Dδ segment, and at least one human Jδ segment, operably linked to a human or non-human TCRδ constant region gene sequence. In some embodiments, the method may optionally further comprise any one of or a combination of the following: (a) inserting into the genome of the non-human animal an unrearranged T cell receptor (TCR) α variable gene locus comprising at least one human Vα segment and at least one human Jα segment, operably linked to a non-human TCRα constant region gene sequence and/or an unrearranged TCRβ variable gene locus comprising at least one human Vβ segment, at least one human Dβ segment, and at least one human Jβ segment, operably linked to a non-human TCRβ constant region gene sequence; (b) introducing into the genome of the non-human animal a first nucleotide sequence encoding a chimeric human/non-human T cell co-receptor polypeptide, a second nucleotide sequence encoding a second chimeric human/non-human T cell co-receptor polypeptide, and/or a third nucleotide sequence encoding a third chimeric human/non-human T cell co-receptor polypeptide, wherein a non-human portion of each chimeric T cell co-receptor polypeptide comprises at least transmembrane and cytoplasmic domains of a non-human T cell co-receptor, and wherein a human portion of each chimeric polypeptide comprises an extracellular portion (or part thereof) of a human T cell co-receptor; (c) placing into the genome a first nucleic acid sequence encoding a first chimeric human/non-human MHC polypeptide, a second nucleic acid sequence encoding a second chimeric human/non-human MHC polypeptide and/or a third nucleic acid sequence encoding a third chimeric human/non-human MHC polypeptide and/or (d) adding into the genome of the non-human animal a β2 microglobulin locus encoding a human or humanized β2 microglobulin polypeptide. In some embodiments, the method may further comprise: (a) inserting into the genome of the non-human animal an unrearranged T cell receptor (TCR) a variable gene locus comprising at least one human Vα segment and at least one human Jα segment, operably linked to a non-human TCRα constant region gene sequence and/or an unrearranged TCRβ variable gene locus comprising at least one human Vβ segment, at least one human Dβ segment, and at least one human Jβ segment, operably linked to a non-human TCRβ constant region gene sequence; and optionally the method may further comprise (b) introducing into the genome of the non-human animal a first nucleotide sequence encoding a chimeric human/non-human T cell co-receptor polypeptide, a second nucleotide sequence encoding a second chimeric human/non-human T cell co-receptor polypeptide, and/or a third nucleotide sequence encoding a third chimeric human/non-human T cell co-receptor polypeptide, wherein a non-human portion of each chimeric T cell co-receptor polypeptide comprises at least transmembrane and cytoplasmic domains of a non-human T cell co-receptor, and wherein a human portion of each chimeric polypeptide comprises an extracellular portion (or part thereof) of a human T cell co-receptor; (c) placing into the genome a first nucleic acid sequence encoding a first chimeric human/non-human MHC polypeptide, a second nucleic acid sequence encoding a second chimeric human/non-human MHC polypeptide and/or a third nucleic acid sequence encoding a third chimeric human/non-human MHC polypeptide and (d) adding into the genome of the non-human animal a β2 microglobulin locus encoding a human or humanized β2 microglobulin polypeptide. In some embodiments, the steps of introducing, inserting and/or placing comprise targeting sequences encoding the extracellular domain(s) of the T cell co-receptor, the variable domain(s) of the TCR and optionally the TCR constant region gene sequences, the extracellular domain(s) of the MHC polypeptide, or a portion of the β2 microglobulin and replacing them with sequences encoding human T cell co-receptor extracellular domain(s), human TCR variable domains and optionally the human TCR constant domains, human MHC extracellular domain(s), and/or a human portion of the β2 microglobulin, respectively.

In other embodiments, introducing, inserting, placing and/or adding may comprise breeding, e.g., mating, animals of the same species. In other embodiments, introducing, inserting, placing and/or adding comprises sequential homologous recombination in ES cells. In some embodiments, the ES cells are derived from non-human animals genetically modified to comprise one or more, but not all, of the genetic modifications desired, and homologous recombination in such ES cells completes the genetic modification. In other embodiments, introducing, inserting, placing and/or adding may comprise a combination of breeding and homologous recombination in ES cells, e.g., breeding an animal to another (or more) animal of the same species, wherein some or all of the animals may be generated from ES cells genetically modified via a single homologous recombination or sequential homologous recombination events, and wherein some ES cell may be isolated from a non-human animal comprising one or more of the genetic modifications disclosed herein.

In some embodiments, the method utilizes a targeting construct made using VELOCIGENE® technology, introducing the construct into ES cells, and introducing targeted ES cell clones into a mouse embryo using VELOCIMOUSE® technology, as described in the Examples. Targeting construct may comprise 5′ and/or 3′ homology arms that target the endogenous sequence to be replaced, an insert sequence (that replaces the endogenous sequence) and one or more selection cassettes. A selection cassette is a nucleotide sequence inserted into a targeting construct to facilitate selection of cells (e.g., ES cells) that have integrated the construct of interest. A number of suitable selection cassettes are known in the art. Commonly, a selection cassette enables positive selection in the presence of a particular antibiotic (e.g., Neo, Hyg, Pur, CM, SPEC, etc.). In addition, a selection cassette may be flanked by recombination sites, which allow deletion of the selection cassette upon treatment with recombinase enzymes. Commonly used recombination sites are loxP and Frt, recognized by Cre and Flp enzymes, respectively, but others are known in the art. A selection cassette may be located anywhere in the construct outside the coding region. In one embodiment, the selection cassette is located at the 5′ end the human DNA fragment. In another embodiment, the selection cassette is located at the 3′ end of the human DNA fragment. In another embodiment, the selection cassette is located within the human DNA fragment. In another embodiment, the selection cassette is located within an intron of the human DNA fragment. In another embodiment, the selection cassette is located at the junction of the human and mouse DNA fragment.

In some embodiments, the method for making a genetically modified non-human animal results in the animal whose genome comprises a human or humanized unrearranged TCR locus (e.g., a human or humanized unrearranged TCRγ and/or TCRδ locus, and optionally a human or humanized TCRα and/or TCRβ, locus). In one embodiment, a method for making a genetically modified non-human animal (e.g., rodent, e.g., mouse or rat) that expresses a T cell receptor comprising a human variable region and a human or endogenous TCR constant domain on a surface of a T cell is provided, wherein the method comprises inserting, e.g., replacing, in a first non-human animal an endogenous non-human TCRγ variable gene locus with an unrearranged humanized TCRγ variable gene locus comprising at least one human Vγ segment and at least one human Jγ segment, wherein the humanized TCRγ variable gene locus is operably linked to a human or endogenous TCRγ constant region gene sequence; inserting, e.g., replacing, in a second non-human animal an endogenous non-human TCRδ variable gene locus with an unrearranged humanized TCRδ variable gene locus comprising at least one human Vδ segment, one human Dδ segment, and one human Jδ segment, wherein the humanized TCRδ variable gene locus is operably linked to a human or endogenous TCRδ constant region gene sequence; and breeding the first and the second non-human animal to obtain a non-human animal that expresses a T cell receptor comprising a human or humanized γ/δ TCR. In some embodiments, the method for making a genetically modified non-human animal results in the animal whose genome comprises a human unrearranged TCR locus (e.g., a human unrearranged TCRγ and/or TCR locus, and optionally a human unrearranged TCRα and/or TCRβ, locus). In one embodiment, a method for making a genetically modified non-human animal (e.g., rodent, e.g., mouse or rat) that expresses a T cell receptor comprising a human variable region and human constant domain on a surface of a T cell is provided, wherein the method comprises inserting, e.g., replacing, in a first non-human animal an endogenous non-human TCRγ gene locus with an unrearranged human TCRγ gene locus comprising at least one human Vγ segment and at least one human Jγ segment operably linked to a human TCRγ constant region gene sequence (e.g., wherein the method further comprises replacing a nucleotide sequence comprising an endogenous TCRγ constant region gene sequence (e.g., an endogenous Trgc1 constant region gene sequence, an endogenous Trgc2 constant region gene sequence, an endogenous Trgc3 constant region gene sequence, and/or an endogenous Trgc4 constant region gene sequence) with a nucleotide sequence comprising a human TCRγ constant region gene sequence, (e.g., a human TRGC1 constant region sequence and/or a human TRGC2 constant region sequence); inserting, e.g., replacing, in a second non-human animal an endogenous non-human TCRδ gene locus with an unrearranged human TCRδ variable gene locus comprising at least one human Vδ segment, one human Dδ segment, and one human Jδ segment operably linked to a human TCRδ constant region gene sequence (e.g., wherein the method further comprises replacing an endogenous TCRδ constant region gene sequence with a human TCRδ constant region gene sequence); and breeding the first and the second non-human animal to obtain a non-human animal that expresses a T cell receptor comprising a human γ/δ TCR. In some embodiments, the second non-human animal comprises a human or humanized TCR α locus prior to and/or in addition to the human TCR δ locus.

In some embodiments, the method for making a genetically modified non-human animal results in the animal whose genome comprises a human unrearranged TCR locus (e.g., a human unrearranged TCRγ and/or TCRδ locus, and optionally a human or humanized unrearranged TCRα and/or TCRβ locus). In one embodiment, a method for making a genetically modified non-human animal (e.g., rodent, e.g., mouse or rat) that expresses a T cell receptor comprising a human variable domain and a human constant domain on a surface of a T cell is provided, wherein the method comprises inserting, e.g., replacing, in a first non-human animal an endogenous non-human TCRγ sequence comprising at least one endogenous Vγ segment (e.g., all endogenous Vγ segments), at least one endogenous Jγ segment (e.g., all endogenous Jγ segments), and at least one endogenous Cγ gene (e.g., all endogenous Cγ genes) with an unrearranged human TCRγ variable gene locus comprising at least one human Vγ segment (e.g., all human Vγ segments), at least one human Jγ segment (e.g., all human Jγ segments), and at least one human Cγ gene (e.g., all human Cγ genes); inserting, e.g., replacing in a second non-human animal an endogenous non-human TCR sequence comprising at least one endogenous Vδ segment (e.g., all endogenous Vδ segments), at least one endogenous Dδ segment (e.g., all endogenous Dδ segments), at least one endogenous Jδ segments (e.g., all endogenous Jδ segments), and an endogenous Cδ with an unrearranged humanized TCRδ gene locus comprising at least one human Vδ segment (e.g., all human Vδ segments), at least one human Dδ segment (e.g., all human Dδ segments), at least one human Jδ segment (e.g., all human Jδ segments), and a human Cδ gene; and breeding the first and the second non-human animal to obtain a non-human animal that expresses a T cell receptor comprising a human γ/δ TCR. In some embodiments, the second non-human animal comprises a human or humanized TCR α locus prior to and/or in addition to the human TCR δ locus.

In some embodiments, the methods further comprise inserting, e.g., replacing, in a first non-human animal an endogenous non-human TCRα variable gene locus with an unrearranged humanized TCRα variable gene locus comprising at least one human Vα segment and at least one human Jα segment, wherein the humanized TCRα variable gene locus is operably linked to endogenous TCRα constant region gene sequence; inserting, e.g., replacing in a second non-human animal an endogenous non-human TCRβ variable gene locus with an unrearranged humanized TCRβ variable gene locus comprising at least one human Vβ segment, one human Dβ segment, and one human Jβ segment, wherein the humanized TCRβ variable gene locus is operably linked to endogenous TCRβ constant region gene sequence; and breeding the first and the second non-human animal to obtain a non-human animal that expresses a T cell receptor comprising a human variable region and a non-human constant region gene sequence. In other embodiments, the invention provides methods of making a genetically modified non-human animal whose genome comprises a humanized unrearranged TCRα locus, or a non-human animal whose genome comprises a humanized unrearranged TCRβ locus.

In various embodiments, the replacements are made at the endogenous loci. In various embodiments, the method comprises progressive humanization strategy, wherein a construct comprising additional variable region segments is introduced into ES cells at each subsequent step of humanization, ultimately resulting in a mouse comprising a complete repertoire of human variable region segments and fully human constant region gene sequences (see, e.g., FIGS. 2 and 3).

Some method embodiments described herein may also comprise (1) replacing an endogenous non-human (e.g., mouse) tcrbdj1 sequence with a nucleic acid sequence comprising a human TRBD1 and human TRBJ1-1 to TRBJ1-6 gene segments and non-human (e.g., mouse) tcrbdj1 non-coding sequences (including non-coding recombination signal sequences (RSSs) and other non-intergenic sequences), where the human TRBD1 and human TRBJ1-1 to TRBJ1-6 gene segments flank the same non-human (e.g., mouse) tcrbdj1 TCR non-coding sequences as are normally flanked by non-human (e.g., mouse) Trbd1 and non-human (e.g., mouse) Trbj1-1 to Trbj1-6 gene segments and/or (2) replacing an endogenous non-human (e.g., mouse) tcrbdj2 sequence with a nucleic acid sequence comprising a human TRBD2 and human TRBJ2-1 to TRBJ2-7 gene segments and mouse tcrbdj2 non-coding sequences, where the human TRBD2 and human TRBJ2-1 to TRBJ2-7 gene segments flank the same mouse Tcrbdj2 non-coding sequences as are normally flanked by the mouse Trbd2 and mouse Trbj2-1 to Trbj2-7 gene segments. In some embodiments, such replacements result in operable linkage of the nucleic acid sequence that comprises a human TRBD1 and human TRBJ1-1 to TRBJ1-6 gene segments and non-human (e.g., mouse) tcrbdj1 non-coding sequences (including non-coding recombination signal sequences (RSSs) and other non-intergenic sequences) to a non-human (e.g., mouse) tcrbc1 constant region gene sequence and/or the operable linkage of the sequence that comprises a human TRBD2 and human TRBJ2-1 to TRBJ2-7 gene segments and mouse TCRBDJ2 non-coding sequences (including non-coding recombination signal sequences (RSSs) and other non-intergenic sequences) to a non-human (e.g., mouse) tcrbc2 constant region gene sequence, respectively (See, FIG. 8C). In such embodiments, resulting mice may comprise one to a complete repertoire of human TCRB variable region segments operably linked to TCRBDJ1 and TCRBDJ2 clusters in which endogenous TCRB non-coding sequences, e.g., non-coding DNA (e.g., non-coding recombination signal sequences (RSSs) and other non-coding intergenic sequences) that separates gene segments, may be retained.

The disclosure also provides a method of modifying a TCR variable gene locus (e.g., TCRα, TCRβ, TCRδ, and/or TCRγ gene locus) of a non-human animal to express a human or humanized TCR protein described herein. In one embodiment, the invention provides a method of modifying a TCR variable gene locus to express a human or humanized TCR protein on a surface of a T cell wherein the method comprises inserting, e.g., replacing, in a non-human animal an endogenous non-human TCR variable gene locus with an unrearranged humanized TCR variable gene locus. In one embodiment wherein the TCR variable gene locus is a TCRγ variable gene locus, the unrearranged humanized TCR variable gene locus comprises at least one human Vγ segment and at least one human Jγ segment, optionally operably linked to a human Cγ gene. In one embodiment wherein the TCR variable gene locus is a TCR variable gene locus, the unrearranged humanized TCR variable gene locus comprises at least one human Vδ segment, at least one human Dδ segment, and at least one human Jδ segment. In various aspects, the unrearranged humanized TCR variable gene locus is operably linked to the corresponding human TCR constant region gene sequence.

Thus, nucleotide constructs for generating genetically modified animals comprising humanized TCR variable region genes are also provided. In one aspect, the nucleotide construct comprises: 5′ and 3′ homology arms, a human DNA fragment comprising human TCR variable region gene segment(s) and human TCR constant region gene sequence(s), and a selection cassette flanked by recombination sites.

In one aspect, at least one homology arm is a non-human homology arm and it is homologous to non-human TCR locus (e.g., non-human TCRγ). In one aspect, one or more homology arms is a human homology arm, and it is homologous to a human TCR locus (e.g., a human TCRα locus) within a non-human animal genome.

Various exemplary embodiments of the humanized loci described herein are presented in the figures and described in the examples.

Upon completion of gene targeting, ES cells or genetically modified non-human animals are screened to confirm successful incorporation of exogenous nucleotide sequence of interest or expression of exogenous polypeptide. Numerous techniques are known to those skilled in the art, and include (but are not limited to) Southern blotting, long PCR, quantitative PCR (e.g., real-time PCR using TAQMAN®), fluorescence in situ hybridization, Northern blotting, flow cytometry, Western analysis, immunocytochemistry, immunohistochemistry, etc. In one example, non-human animals (e.g., mice) bearing the genetic modification of interest can be identified by screening for loss of mouse allele and/or gain of human allele using a modification of allele assay described in Valenzuela et al. (2003) High-throughput engineering of the mouse genome coupled with high-resolution expression analysis, Nature Biotech. 21(6):652-659. Other assays that identify a specific nucleotide or amino acid sequence in the genetically modified animals are known to those skilled in the art.

In some embodiments, animals are generated herein by breeding. For example, in some embodiments, mice are generated by a method that comprises:
(a) obtaining a first mouse comprising a homozygous replacement of an endogenous genomic sequence comprising endogenous TCR Vγ and Jγ gene segments and an endogenous TCR Cγ gene with the heterologous sequence comprising the unrearranged human TCR Vγ segment and the unrearranged human TCR Jγ segment operably linked with the human TCR γ constant region gene sequence,
(b) obtaining a second mouse comprising a homozygous replacement of an endogenous genomic sequence comprising endogenous TCR Vδ, Dδ, Jδ gene segments and an endogenous TCR Cδ gene with the heterologous sequence comprising the unrearranged human TCR Vδ segment, the unrearranged human TCR Dδ segment, the unrearranged human TCR Jδ segment operably linked to the human TCR δ constant region gene sequence, and
(c) breeding the first mouse and the second mouse to obtain a genetically modified
wherein the genetically modified mouse comprises: mouse,
   (i) the replacement of an endogenous genomic sequence comprising endogenous TCR Vγ and Jγ gene segments and an endogenous TCR Cγ gene with the heterologous sequence comprising the unrearranged human TCR Vγ segment and the unrearranged human TCR Jγ segment operably linked with the human TCR γ constant region gene sequence, and
   (ii) the replacement of an endogenous genomic sequence comprising endogenous TCR Vδ, Dδ, Jδ gene segments and an endogenous TCR Cδ gene with the heterologous sequence comprising the unrearranged human TCR Vδ segment, the unrearranged human TCR Dδ segment, the unrearranged human TCR Jδ segment operably linked to the human TCR δ constant region gene sequence, and
wherein the genetically modified mouse expresses a human TCR γ polypeptide and a human TCR δ polypeptide.
   Using Genetically Modified Non-Human Animals that Mount Substantially Humanized T Cell Immune Responses

The roles of γ/δ T cells in immune defense remain poorly understood. See, e.g., Vermijlen, D. et al. (2017) Seminars in Cell and Developmental Biol. 84:75-86. However, several features outlined above hold promise for therapeutic application. A major barrier to T cell therapies derived from α/β T cells is MHC restriction of target antigens, which limits the applicability of any engineered α/β TCR to patients with appropriate MHC haplotype. In addition, many α/β T cells are ‘alloreactive’ to foreign MHC, which limits their use as allogeneic cell therapies due to risk of graft versus host disease (GVHD). γ/δ T cells lack these caveats and may be a rapid route to ‘off-the-shelf’ allogeneic therapies requiring less extensive engineering and manipulation to mitigate GVHD risks. In addition, the observed intrinsic anti-tumor and anti-microbial properties of γ/δ T cells—akin to other ‘innate-like’ cells such as natural killer cells—may have further clinical benefit that is being evaluated in various trials. See, e.g., Ferry G. M. and Anderson J. (2002) Exp. Immunol 2:168-79; Park J. H and Lee H. K. (2021) Experimental & Molecular Medicine 53:318-327.

TRG/TRD humanized mice described herein provide for several novel areas of research into γ/δ T cell biology and potential therapies. While published mice have effectively modeled infectious and autoimmune disease responses dominated by α/β T cells (Moore, M. et al. Sci Immunol 6 (2021); doi:10.1126/sciimmunol.abj4026; incorporated herein in its entirety by reference), further humanization of the γ/δ lineage may facilitate modeling of skin and mucosal immune responses. This includes, but is not limited to, research into the role of γ/δ T cells and antigens in inflammatory skin disease, interactions with gut microbiota, and barrier tissue repair and homeostasis. TRG/TRD humanized mice also provide a new tool for investigating and identifying γ/δ T cell antigens and cognate TCRs, which are poorly characterized compared to their α/β counterparts. For instance, identification of antigen/TCR interactions that are enriched on tumor cells may lead γ/δ based anti-tumor cell therapies devoid of the MHC restriction that limits existing TCR therapies. Moreover, since graft-versus-host disease is caused by alloreactive α/β T cell receptor, γ/δ T cells are becoming increasingly interesting in allogeneic hematopoietic stem cell transplantation, and clinical strategies to exploit the full function of these lymphocytes have been and are being developed, which strategies may include in vivo activation of γ/δ T cells or subsets after transplantation by certain drugs or antibodies or the ex vivo expansion and manipulation of either patient-derived or donor-derived γ/δ T cells and their subsets and the adoptive transfer of the ex vivo—activated lymphocytes. Handgretinger, R. and Schilbach, K. (2018) Blood 131:1063-72, incorporated herein by reference. As such, TRG/TRD humanized mice as described herein may be useful tools in preclinical investigation of new approaches aimed to expand γ/δ T cells, direct the same to tumors, and/or to expand γ/δ T cells in vivo or ex vivo.

Also described herein are embodiments where humanized TRA/D and TRG loci are introduced into mice with humanized TRB loci and other components of T cell immunity, including loci of TCR co-receptors (CD4 and CD8) and MHC. These mice therefore bear full humanization of both T cell lineages to aid in studies involving cellular immunity.

The genetically modified non-human animals, e.g., rodents, e.g., mice or rats, described herein provide non-MHC restricted γ/δ responses, and optionally, when substantially all of the components of the complex are human or humanized, either humanized CD4 and MHC II or humanized CD8 and MHC I (and β2 microglobulin), or both, present peptides to α/β T cells (CD4+ or CD8+ T cells, respectively) in a human-like manner. Thus, the genetically modified non-human animals of the invention can also be used to study the function of a human immune system in the humanized animal; for identification of antigens and antigen epitopes that elicit immune response (e.g., T cell epitopes, e.g., unique human cancer epitopes), e.g., for use in vaccine development; for identification of high affinity T cells to human pathogens or cancer antigens (i.e., T cells that bind to antigen in the context of human MHC I complex with high avidity), e.g., for use in adaptive T cell therapy and “innate” T cell therapy; for evaluation of vaccine candidates and other vaccine strategies; for studying human autoimmunity; for studying human infectious diseases; and otherwise for devising better therapeutic strategies based on human TCR expression.

Thus, in various embodiments, the genetically engineered animals of the present invention are useful, among other things, for evaluating the capacity of an antigen to initiate an immune response in a human, and for generating a diversity of antigens and identifying a specific antigen that may be used in human vaccine development.

In one aspect, a method for determining whether a peptide will provoke a cellular immune response in a human is provided, comprising exposing a genetically modified non-human animal as described herein to the peptide, allowing the non-human animal to mount an immune response, and detecting in the non-human animal a TCR (e.g., a γ/δ TCR) that binds a sequence of the peptide, by itself (or for α/β TCR, presented by a chimeric human/non-human MHC I or II molecule) as described herein.

In one aspect, a method for identifying a candidate agent that expands and/or activates γ/δ T cells is described, comprising administering the candidate agent to a non-human animal as described herein (or an in vitro composition comprising γ/δ T cells isolated from a non-human animal as described herein) and measuring the level of γ/δ T cell expansion and/or activation, wherein an increased level of γ/δ T cell expansion and/or activation identifies the candidate agent as one that can expand and/or activate γ/δ T cells. In some embodiments, the candidate agent is a tumor associated antigen. In some embodiments, the candidate agent is an antibody.

In one aspect, a method of stimulating and/or activating γ/δ T cells (e.g., of Vδ2 γ/δ T cells) comprises administering an atninobisphosphonates (e.g., zoledronate, pamidronate, risedronate) to a non-human animal as described herein. See, e.g., Latha et al. (2014) Front. Immunol. Vol. 5 DOI=10.3389/fimmu.2014.00571, incorporated herein in its entirety by reference. Aminobisphophonates act as inhibitors of the farnesyl pyrophosphate synthase (FPPS) in the mevalonate pathway to provoke the accumulation of isopentenyl pyrophosphates (IPPs), and thus, activate γ/δ T cells in vivo. See, e.g., Park et al. (2021) Vol. 8 doi.org/10.3389/fchem.2020.612728.

In one aspect, a method for identifying a human T cell epitope is provided, comprising exposing a non-human animal as described herein to an antigen comprising a putative T cell epitope, allowing the non-human animal to mount an immune response, isolating from the non-human animal an T cell that binds the epitope, which may or may not be MHC class I- or MHC class II-restricted, and identifying the epitope bound by said T cell.

In one aspect, a method is provided for identifying an antigen that generates a T cell response in a human, comprising exposing a putative antigen to a mouse as described herein, allowing the mouse to generate an immune response, and identifying the antigen recognized by the T cell, which may or may not be presented by the HLA class I- or class II-restricted molecule.

In one aspect, a method is provided for determining whether a putative antigen contains an epitope that upon exposure to a human immune system may require or generate an HLA class I- or class II-restricted immune response, comprising exposing a mouse as described herein to the putative antigen and measuring a γ/δ or an antigen-specific HLA class I- or HLA class II-restricted immune response in the mouse, respectively.

In addition, the genetically engineered non-human animals described herein may be useful for identification of T cell receptors, e.g., high-avidity T cell receptors, that recognize an antigen of interest, e.g., a tumor or another disease antigen. The method may comprise: exposing the non-human animal described herein to an antigen, allowing the non-human animal to mount an immune response to the antigen, isolating from the non-human animal a T cell comprising a T cell receptor that binds the antigen (whether or not presented by a human or humanized MHC I or MHC II), and determining the sequence of said T cell receptor. In some embodiments, the methods comprise exposing the non-human animal described herein to an antigen, allowing the non-human animal to mount an immune response to the antigen, isolating from the non-human animal a γ/δ T cell comprising a γ/δ T cell receptor that binds the antigen in the absence of an MHC. Alternatively to exposure to an antigen, γ/δ T cells, similar to natural killer (NK) cells, may respond to stress-induced self-ligands such as major histocompatibility complex class I—related chain A and B (MICAS) and UL16-binding proteins (ULBPs) via activation of the NKG2D receptor (via NKG2D ligands) expressed on γ/δ T cells and via the Vδ1 receptor. In addition, γ/δ T cells also express pattern-recognition receptors, such as toll-like receptors, which augment their antitumor activity. It has also been shown that γ/δ T cells express natural cytotoxicity receptors NKp30 and NKp44 and that γ/δ T cells can kill lymphoid leukemia cell lines and leukemic blasts from patients with chronic myeloid leukemia through NKp30.18. Finally, with the activating receptor DNAM-1, γ/δ T cells efficiently target nectin-2 (CD112) and poliovirus receptor (CD155) positive acute myeloid leukemia and multiple myeloma cells.

Non-human animals expressing a diverse repertoire of functional human TCR V(D)J gene segments may be useful for the study of human diseases. Accordingly, in one embodiment, the genetically engineered non-human animals described herein may express a TCR repertoire substantially similar to a TCR repertoire expressed in a human, e.g., the TCR repertoire of a non-human animal disclosed herein may be derived from at least about 50%, at least about 60%, at least about 70%, at least about 75%, at least about 80%, at least about 85%, at least about 90%, at least about 95%, at least about 97%, or at least about 99% of all functional human TCR α, TCR β, TCRγ and/or TCRδ gene segments.

In addition to the ability to identify antigens and antigen epitopes from human pathogens or neoplasms, the genetically modified animals of the invention can be used to identify autoantigens of relevance to human autoimmune diseases, e.g., type I diabetes, multiple sclerosis, etc. Also, the genetically modified animals of the invention can be used to study various aspects of human autoimmune disease and may be utilized as autoimmune disease models.

In various embodiments, the genetically modified non-human animals of the invention make T cells with human or humanized TCR molecules on their surface, and as a result, would recognize peptides in a human-like manner, optionally when the peptides are presented to them by MHC complexes. The genetically modified non-human animals described herein may be used to study the development and function of human T cells and the processes of immunological tolerance; to test human vaccine candidates; to generate TCRs with certain specificities for TCR gene therapy; to generate TCR libraries to disease associated antigens (e.g., tumor associated antigens (TAAs); etc.

There is a growing interest in T cell therapy in the art, as T cells (e.g., cytotoxic T cells) can be directed to attack and lead to destruction of antigen of interest, e.g., viral antigen, bacterial antigen, tumor antigen, etc., or cells that present it. Initial studies in cancer T cell therapy aimed at isolation of tumor infiltrating lymphocytes (TILs; lymphocyte populations in the tumor mass that presumably comprise T cells reactive against tumor antigens) from tumor cell mass, expanding them in vitro using T cell growth factors, and transferring them back to the patient in a process called adoptive T cell transfer. See, e.g., Restifo et al. (2012) Adoptive immunotherapy for cancer: harnessing the T cell response, Nature Reviews 12:269-81; Linnermann et al. (2011) T-Cell Receptor Gene Therapy: Critical Parameters for Clinical Success, J. Invest. Dermatol. 131:1806-16. However, success of these therapies has thus far been limited to melanoma and renal cell carcinoma; and the TIL adoptive transfer is not specifically directed to defined tumor associated antigens (TAAs). Linnermann et al., supra.

Attempts have been made to initiate TCR gene therapy where T cells are either selected or programmed to target an antigen of interest, e.g., a TAA. Current TCR gene therapy relies on identification of sequences of TCRs that are directed to specific antigens, e.g., tumor associated antigens. For example, Rosenberg and colleagues have published several studies in which they transduced peripheral blood lymphocytes derived from a melanoma patient with genes encoding TCRα and β chains specific for melanoma-associated antigen MART-1 epitopes, and used resulting expanded lymphocytes for adoptive T cell therapy. Johnson et al. (2009) Gene therapy with human and mouse T-cell receptors mediates cancer regression and targets normal tissues expressing cognate antigen, Blood 114:535-46; Morgan et al. (2006) Cancer Regression in Patients After Transfer of Genetically Engineered Lymphocytes, Science 314:126-29. The MART-1 specific TCRs were isolated from patients that experienced tumor regression following TIL therapy. However, identification of such TCRs, particularly high-avidity TCRs (which are most likely to be therapeutically useful), is complicated by the fact that most tumor antigens are self-antigens, and TCRs targeting these antigens are often either deleted or possess suboptimal affinity, due primarily to immunological tolerance.

In various embodiments, the present invention solves this problem by providing genetically engineered non-human animals comprising in their genome an unrearranged human TCR variable gene locus. The non-human animal described herein is capable of generating T cells with a diverse repertoire of humanized T cell receptors. Thus, the non-human animals described herein may be a source of a diverse repertoire of humanized T cell receptors, e.g., high-avidity humanized T cell receptors for use in adoptive T cell transfer.

Thus, in one embodiment, the present invention provides a method of generating a T cell receptor to a human antigen comprising immunizing a non-human animal (e.g., a rodent, e.g., a mouse or a rat) described herein with an antigen of interest, allowing the animal to mount an immune response, isolating from the animal an activated T cell with specificity for the antigen of interest, and determining the nucleic acid sequence of the T cell receptor expressed by the antigen-specific T cell.

In one embodiment, the invention provides a method of producing a human T cell receptor specific for an antigen of interest (e.g., a disease-associated antigen) comprising immunizing a non-human animal described herein with the antigen of interest; allowing the animal to mount an immune response; isolating from the animal a T cell reactive to the antigen of interest; determining a nucleic acid sequence of a human TCR variable region expressed by the T cell; cloning (a) the human TCR variable region into a nucleotide construct comprising a nucleic acid sequence of a human TCR constant region gene sequence such that the human TCR variable region is operably linked to the human TCR constant region gene sequence or, where the non-human animal comprises a fully human TCR locus (b) cloning the human TCR variable region operably linked to the human TCR constant region sequence into a nucleotide construct; and expressing from the construct a human T cell receptor specific for the antigen of interest. In one embodiment, the steps of isolating a T cell, determining a nucleic acid sequence of at least the human TCR variable region expressed by the T cell, cloning the TCR encoding sequence into a nucleotide construct, and expressing a human T cell receptor are performed using standard techniques known to those of skill the art.

In one embodiment, the nucleotide sequence encoding a T cell receptor specific for an antigen of interest is expressed in a cell. In one embodiment, the cell expressing the TCR is selected from a CHO, COS, 293, HeLa, PERC.6™ cell, etc.

The antigen of interest may be any antigen that is known to cause or be associated with a disease or condition, e.g., a tumor associated antigen; an antigen of viral, bacterial or other pathogenic origin; etc. Many tumor-associated antigens are known in the art. A selection of tumor associated antigens is presented in Cancer Immunity (A Journal of the Cancer Research Institute) Peptide Database (archive.cancerimmunity.org/peptidedatabase/Tcellepitopes.htm). In some embodiments of the invention, the antigen of interest is a human antigen, e.g., a human tumor associated antigen. In some embodiments, the antigen is a cell type-specific intracellular antigen, and a T cell receptor is used to kill a cell expressing the antigen.

In one embodiment, provided herein is a method of identifying a T cell with specificity against an antigen of interest, e.g., a tumor associated antigen, comprising immunizing a non-human animal described herein with the antigen of interest, allowing the animal to mount an immune response, and isolating from the non-human animal a T cell with specificity for the antigen.

The present invention provides new methods for adoptive T cell therapy. Thus, provided herein is a method of treating or ameliorating a disease or condition (e.g., a cancer) in a subject (e.g., a mammalian subject, e.g., a human subject) comprising immunizing a non-human animal described herein with an antigen associated with the disease or condition, allowing the animal to mount an immune response, isolating from the animal a population of antigen-specific T cells, and infusing isolated antigen-specific T cells into the subject. In one embodiment, the invention provides a method of treating or ameliorating a disease or condition in a human subject, comprising immunizing the non-human animal described herein with an antigen of interest (e.g., a disease- or condition-associated antigen, e.g., a tumor associated antigen), allowing the animal to mount an immune response, isolating from the animal a population of antigen-specific T cells, determining the nucleic acid sequence of a T cell receptor, (e.g., a first and/or second nucleic acid sequence encoding the human rearranged TCRδ variable region gene or a TCRγ variable region gene or a third and/or fourth nucleic acid sequence encoding the human rearranged TCRα and/or human rearranged TCRβ variable region gene) expressed by the antigen-specific T cells, cloning the nucleic acid sequence of the T cell receptor, e.g., the first, second, third and/or fourth nucleic acid sequence into an expression vector (e.g., a retroviral vector), introducing the vector into T cells derived from the subject such that the T cells express the antigen-specific T cell receptor, and infusing the T cells into the subject. In one embodiment, the T cell receptor nucleic acid sequence does not need to be further humanized (e.g., since some animal embodiments herein comprise fully human TRD and TRG loci). In one embodiment, the T cell receptor nucleic acid sequence is further humanized prior to introduction into T cells derived from the subject, e.g., any sequence encoding a non-human constant region gene sequence is modified to further resemble a human TCR constant region gene sequence (e.g., the non-human constant region gene sequence is replaced with a human constant region gene sequence). In some embodiments, the disease or condition is cancer. In some embodiments, an antigen-specific T cell population is expanded prior to infusing into the subject. In some embodiments, the subject's immune cell population is immunodepleted prior to the infusion of antigen-specific T cells. In some embodiments, the antigen-specific TCR is a high avidity TCR, e.g., a high avidity TCR to a tumor associated antigen. In some embodiments, the T cell is a cytotoxic T cell. In other embodiments, the disease or condition is caused by a virus or a bacterium.

In another embodiment, a disease or condition is an autoimmune disease. T_{REG} cells are a subpopulation of T cells that maintain tolerance to self-antigens and prevent pathological self-reactivity. Thus, also provided herein are methods of treating autoimmune disease that rely on generation of antigen-specific T_{REG} cells in the non-human animal of the invention described herein.

Also provided herein is a method of treating or ameliorating a disease or condition (e.g., a cancer) in a subject comprising introducing the cells affected by the disease or condition (e.g., cancer cells) from the subject into a non-human animal, allowing the animal to mount an immune response to the cells, isolating from the animal a population of T cells reactive to the cells, determining the nucleic acid sequence of a T cell receptor variable domain expressed by the T cells, cloning the T cell receptor variable domain encoding sequence into a vector (e.g., in-frame to a promoter sequence and, if applicable, operably linked to a human TCR constant region gene sequence), introducing the vector into T cells derived from the subject, and infusing the subject's T cells harboring the T cell receptor into the subject.

Also provided herein is the use of a non-human animal as described herein to make nucleic acid sequences encoding human TCR variable domains (e.g., TCR γ and/or δ variable domains) or encoding fully human TCR polypeptides (e.g., TCR γ and/or δ polypeptides). In one embodiment, a method is provided for making a nucleic acid sequence encoding a human TCR variable domain, comprising immunizing a non-human animal as described herein with an antigen of interest, allowing the non-human animal to mount an immune response to the antigen of interest, and obtaining therefrom a nucleic acid sequence encoding a human TCR variable domain that binds the antigen of interest. In one embodiment, the method further comprises making a nucleic acid sequence encoding a human TCR variable domain, that is optionally operably linked to a non-human TCR constant domain, comprising isolating a T cell from a non-human animal described herein and obtaining therefrom the nucleic acid sequence encoding the TCR variable domain, optionally linked to the non-human TCR constant domain, and cloning the nucleic acid sequence(s) encoding the TCR variable domain (e.g., a first, second, third or fourth nucleic acid sequence respectively encoding a human rearranged TCRγ variable region gene, human rearranged TCRδ variable region gene, TCRα variable region gene or a TCRβ variable region gene) in-frame, e.g., with a promoter with an appropriate human constant region unless already present due to the nature of the genetic modifications of the mouse (e.g., a human TCRγ constant region gene sequence, human TCRδ constant region gene sequence, TCRα constant region gene sequence, or a TCRβ variable region gene sequence, respectively).

Thus, provided herein are TCR variable region nucleic acid sequences, such as rearranged TCR variable nucleic acid sequences, e.g., rearranged TCRγ, TCRδ, or TCRα/δ variable region nucleic acid sequences, generated in the non-human animals described herein, and encoded respectively by, e.g., a human rearranged Vγ/Jγ gene sequence, a rearranged human VδDδJδ gene sequence, or a rearranged human VαDδJδ gene sequence. Also, provided are TCR variable region amino acid sequences encoded by such rearranged TCR variable region nucleic acid sequences. Such rearranged TCR variable region nucleic acid sequences (TCRγ and/or TCRδ variable region nucleic acid sequences) obtained in the non-human animals described herein, and utilized for various uses described herein, e.g., as a human therapeutic, in a human.

TCRαδ variable region nucleic acid sequences have not been heretofore described. Accordingly, such TCRαδ variable region nucleic acid sequences may be particularly useful in identifying a new class of TCR variable domains and/or interrogating such TCRαδ variable region nucleic acid sequences in the human population. Such TCRαδ variable region nucleic acid sequences and the variable domains encoded therefrom may represent a novel class of therapeutics as a hybrid TCR α/δ variable domain may bind altogether a different class of antigen or bind antigen in a manner distinct from those of prototypical TCR α, TCR β, TCR γ or TCR δ variable domains.

Also provided herein is the use of a non-human animal as described herein to make a human therapeutic, comprising immunizing the non-human animal with an antigen of interest (e.g., a tumor associated antigen), allowing the non-human animal to mount an immune response, obtaining from the animal T cells reactive to the antigen of interest, obtaining from the T cells a nucleic acid sequence(s) encoding a humanized TCR protein or human TCR variable domain that binds the antigen of interest, and employing the nucleic acid sequence(s) encoding a humanized TCR protein or a human TCR variable domain in a human therapeutic.

Thus, also provided is a method for making a human therapeutic, comprising immunizing a non-human animal as described herein with an antigen of interest, allowing the non-human animal to mount an immune response, obtaining, from the animal, T cells reactive to the antigen of interest, obtaining from the T cells a nucleic acid sequence(s) encoding a humanized T cell receptor that binds the antigen of interest, and employing the humanized (or fully human) T cell receptor in a human therapeutic.

In one embodiment, the human therapeutic is a T cell (e.g., a human T cell, e.g., a T cell derived from a human subject) harboring a nucleic acid sequence of interest (e.g., transfected or transduced or otherwise introduced with the nucleic acid of interest) such that the T cell expresses the humanized TCR protein with affinity for an antigen of interest. In one aspect, a subject in whom the therapeutic is employed is in need of therapy for a particular disease or condition, and the antigen is associated with the disease or condition. In one aspect, the T cell is a cytotoxic T cell, the antigen is a tumor associated antigen, and the disease or condition is cancer. In one aspect, the T cell is derived from the subject.

In another embodiment, the human therapeutic is a T cell receptor. In one embodiment, the therapeutic receptor is a soluble T cell receptor. Much effort has been expanded to generate soluble T cell receptors or TCR variable regions for use therapeutic agents. Generation of soluble T cell receptors depends on obtaining rearranged TCR variable regions. One approach is to design single chain TCRs comprising TCRα and TCRβ, and, similarly to scFv immunoglobulin format, fuse them together via a linker (see, e.g., International Application No. WO 2011/044186). The resulting scTv, if analogous to scFv, would provide a thermally stable and soluble form of TCRα/β binding protein. Alternative approaches included designing a soluble TCR having TCRβ constant domains (see, e.g., Chung et al., (1994) Functional three-domain single-chain T-cell receptors, Proc. Natl. Acad. Sci. USA. 91:12654-58); as well as engineering a non-native disulfide bond into the interface between TCR constant domains (reviewed in Boulter and Jakobsen (2005) Stable, soluble, high-affinity, engineered T cell receptors: novel antibody-like proteins for specific targeting of peptide antigens, Clinical and Experimental Immunology 142:454-60; see also, U.S. Pat. No. 7,569,664). Other formats of soluble T cell receptors have been described. The non-human animals described herein may be used to determine a sequence of a T cell receptor that binds with high affinity to an antigen of interest, and subsequently design a soluble T cell receptor based on the sequence.

A soluble T cell receptor derived from the TCR receptor sequence expressed by the non-human animal can be used to block the function of a protein of interest, e.g., a viral, bacterial, or tumor associated protein. Alternatively, a soluble T cell receptor may be fused to a moiety that can kill an infected or cancer cell, e.g., a cytotoxic molecule (e.g., a chemotherapeutic), toxin, radionuclide, prodrug, antibody, etc. A soluble T cell receptor may also be fused to an immunomodulatory molecule, e.g., a cytokine, chemokine, etc. A soluble T cell receptor may also be fused to an immune inhibitory molecule, e.g., a molecule that inhibits a T cell from killing other cells harboring an antigen recognized by the T cell. Such soluble T cell receptors fused to immune inhibitory molecules can be used, e.g., in blocking autoimmunity. Various exemplary immune inhibitory molecules that may be fused to a soluble T cell receptor are reviewed in Ravetch and Lanier (2000) Immune Inhibitory Receptors, Science 290:84-89, incorporated herein by reference.

The present invention also provides methods for studying immunological response in the context of human TCR, including human TCR rearrangement, T cell development, T cell activation, immunological tolerance, etc.

Also provided are methods of testing vaccine candidates. In one embodiment, provided herein is a method of determining whether a vaccine will activate an immunological response (e.g., T cell proliferation, cytokine release, etc.), and lead to generation of effector, as well as memory T cells (e.g., central and effector memory T cells).

γ/δ T cells can exert effective antitumor activity against various solid tumors and hematologic malignancies, such as lymphoma and multiple myeloma. Accordingly, in one aspect, an in vitro preparation is provided that comprises a T cell that bears a γ/δ TCR on its surface and a second cell that expresses a tumor associated antigen that is bound by the γ/δ TCR.

Other uses of the genetically modified animals described herein, i.e., animals comprising a human or humanized T cell co-receptor (e.g., chimeric human/non-human CD4 or CD8), optionally further comprising a human or humanized MHC II or I protein, will be apparent from the present disclosure.

Table 2 provides a brief description of the sequences in the sequence listing.

TABLE 2

| SEQ ID NO | DESCRIPTION |
|---|---|
| 1 | Chimeric human/mouse CD4 polypeptide |
| 2 | Human portion of exemplary chimeric human/mouse CD4 |
| | polypeptide |
| 3 | Exemplary chimeric human/mouse CD8α polypeptide |
| 4 | Human portion of exemplary chimeric human/mouse CD8α |
| | polypeptide |
| 5 | Exemplary chimeric human/mouse CD8β polypeptide |
| 6 | Human portion of exemplary chimeric human/mouse CD8β |
| 7 | 5′ primer for chr14: 22429215-22429411 (GRCh38 |
| | assembly) homology arm |
| 8 | 3′ primer for chr14: 22429215-22429411 (GRCh38 |
| | assembly) homology arm |
| 9 | 5′ primer for chr14: 22429412-22429599 (GRCh38 |
| | assembly) homology arm |
| 10 | 3′ primer for chr14: 22429412-22429599 (GRCh38 |
| | assembly) homology arm |
| 11 | TCRdh1 probe |
| 12 | TCRdh3 probe |
| 13 | 785h1 probe |
| 14 | 785h2 probe |
| 15 | 785h3 probe |
| 16 | 5′ primer for chr13: 19177740-19177875 (GRCh38 |
| | assembly) homology arm |
| 17 | 3′ primer for chr13: 19177740-19177875 (GRCh38 |
| | assembly) homology arm |
| 18 | 5′ primer for chr13: 19357360-19357510 (GRCh38 |
| | assembly) homology arm |
| 19 | 3′ primer for chr13: 19357360-19357510 (GRCh38 |
| | assembly) homology arm |
| 20 | TCRg mTF probe |
| 21 | TCRg mTN probe |
| 22 | TCRg mTV probe |
| 23 | 5′ primer for chr7: 38383439-38383590 (GRCh38 |
| | assembly) homology arm |
| 24 | 3′ primer for chr7: 38383439-38383590 (GRCh38 |
| | assembly) homology arm |
| 25 | 5′ primer for chr7: 38230786-38230960 (GRCh38 |
| | assembly) homology arm |
| 26 | 3′ primer for chr7: 38230786-38230960 (GRCh38 |
| | assembly) homology arm |
| 27 | TCRg hTN probe |
| 28 | TCRg hTV probe |
| 29 | TCRg mTF probe |
| 30 | TCRg mTN probe |
| 31 | TCRg mTV probe |
| 32 | Template switching (TS) primer for reverse transcription |
| 33 | PCR1 forward primer |
| 34 | PCR1 reverse primer TCRd constant |
| 35 | PCR1 reverse primer TCRg constant |
| 36 | PCR2 forward primer |
| 37 | PCR2 reverse primer TCRd constant |
| 38 | PCR2 reverse primer TCRg constant |
| 39 | 5′ primer for backbone of pBACe3.6 homology arm |
| 40 | 3′ primer for backbone of pBACe3.6 homology arm |
| 41 | 5′ primer for chr14: 21548298- 21548510 (GRCh38 |
| | assembly) homology arm |
| 42 | 3′ primer for chr14: 21548298- 21548510 (GRCh38 |
| | assembly) homology arm |

### EXAMPLES

The following examples are provided so as to describe to those of ordinary skill in the art how to make and use methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. The Examples do not include detailed descriptions of conventional methods that would be well known to those of ordinary skill in the art (molecular cloning techniques, etc.). Unless indicated otherwise, parts are parts by weight, molecular weight is average molecular weight, temperature is indicated in Celsius, and pressure is at or near atmospheric.

### Example 1: Generation of Mice with Human TCR Delta and TCR Gamma Loci

### Example 1.1: Genetic Engineering of Mice with Human TCR Delta Locus

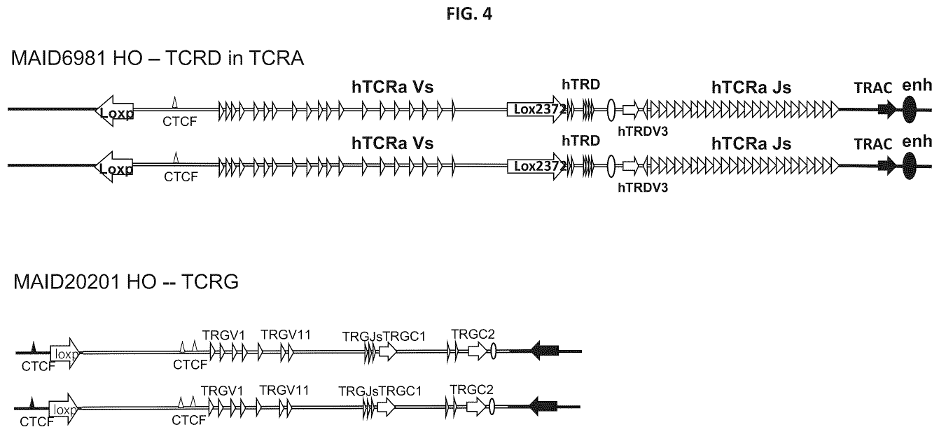

A human TCRD gene locus was introduced into the TCRA locus of a previously humanized TCRA genome sequence MAID1771 (See e.g., FIG. 8A, see also Example 2.1 and FIG. 4A of U.S. Pat. No. 11,259,510, incorporated herein by reference) using VELOCIGENE® technology (see, e.g., U.S. Pat. No. 6,586,251 and Valenzuela et al. (2003) High-throughput engineering of the mouse genome coupled with high-resolution expression analysis. Nat. Biotech. 21(6): 652-659, both incorporated herein by reference). MAID 6316 (VI785), a targeting vector comprising a 101 kb 5′ human homology arm, a 46 kb genomic sequence of human TCRD locus Chr14:22421820-22464666 (GRCh38 assembly) from TRDV2 to TRDC genes, and a 33 kb 3′ human homology arm (comprising human TCRDV3 gene and the majority of human TRAJ genes), was inserted into the human TCRA sequence located in mouse TCRA locus on mouse chromosome 14, See FIG. 2.

In detail, a Lox2372-Ub-Hyg cassette was added into human BAC clone CTD-3170E13 (Thermo Fisher Scientific) by bacterial homologous recombination (BHR). First, human homology arms were made by PCR amplification using CTD-3170E13 as the template, with primers utilized in Table 3 below:

TABLE 3

| | | | Coordinates |
|---|---|---|---|
| Homology | 5′ primer | 3′ primer | (GRCh38 |
| Arm | (SEQ ID NO) | (SEQ ID NO) | assembly) |
| 5′; 197 bp | CCACATTCTGTGGACTGGTG | ATTGATTTCAGCTCCACAACTG | chr14:22429215- |
| | (SEQ ID NO: 7) | (SEQ ID NO: 8) | 22429411 |
| 3′; 188 bp | CATGTGTTCACCTCAGTGGG | TACCTTAGGCCGTGTGATCC | chr14:22429412- |
| | (SEQ ID NO: 9) | (SEQ ID NO: 10) | 22429599 |

To make the targeting vector (designated MAID6316), a construct comprising human 5′ homology arm comprising the sequence amplified using primers depicted in Table 3, a Hygromycin-resistance cassette flanked by Lox2372, and a 3′ homology arm comprising the sequence amplified using primers depicted Table 3, was inserted using bacterial homologous recombination (BHR) on human BAC clone CTD-3170E13.

The targeting vector MAID 6316 contained, from 5′ to 3′, 180 kb sequence including 101 kb 5′ homology arm and a sequence including the lox2372-Ub-Hyg-lox2372 cassette and 46 kb human genomic sequence from the human TCRD locus from TRDV2 to TRDC gene; 33 kb 3′ homology arm, and a chloramphenicol resistance cassette. See, FIG. 2. Final clone was selected based on CM/Hygromycin resistance.

MAID6316 targeting vector was electroporated into mouse embryonic stem (ES) cells comprising a humanized TRA locus, which is a selection cassette-deleted version of the locus depicted as MAID1771 in FIG. 2. (See also Example 2.1 and FIG. 4A of U.S. Pat. No. 11,259,510, incorporated herein by reference). Targeted homologous recombination resulted in insertion of human TCRD sequence including 42.8 kb of human sequence (GRCh38 coordinates Chr14:22421820-22464666) from TRDV2 to TRDC genes. Successful integration was confirmed by a modification of allele (MOA) assay as described, e.g., in Valenzuela et al, supra. Probes used for the MOA assay for detecting presence of human TCRD sequences (gain of allele) are depicted in Table 4 below, and coordinates according to human genome assembly GRCh38 are also included. The selection cassette was subsequently removed by transient expression of CRE recombinase in the ES cell clone.

TABLE 4

| | | Coordinates |
|---|---|---|
| Probe | | (GRCh38 |
| Name | Probe (5′ to 3′) | assembly) |
| TCRdh1 | CCTGACCACTGCTGTTTGTTCCAGA | chr14: |
| | (SEQ ID NO: 11) | 22463750- |
| | | 22463774 |
| TCRdh3 | CAGCAAAGAAACATCAAGGCTCACAGGA | chr14: |
| | (SEQ ID NO: 12) | 22422631- |
| | | 22422658 |

To make the final targeting vector MAID 6980, which added the CTCF binding element from the 5′ of TCRA locus, human BAC clone RP11-720H19 (Thermofisher) was modified. A first construct comprising a human 5′ homology arm, a Neomycin-resistance cassette flanked by ICeu1 and SfaAI, and a 3′ homology was inserted using bacterial homologous recombination (BHR) at a location that was 5′ of the CTCF sequence in human BAC clone RP11-720H19 to create a “modified human BAC clone RP11-720H19”. Each of the human 5′ homology arm and 3′ homology arm of this first construct comprised a sequence amplified using the primers provided in Table 5. A second construct, comprising the same 5′ mouse arm previously used in MAID1771, followed by floxed Hygromycin-resistance cassette, and flanked by restriction sites of ICeu1 and SfaAI, was cloned into the modified human BAC clone RP11-720H19 by restriction digestion to replace the sequence of the Neo cassette. This resulted in the targeting vector designated MAID6980.

TABLE 5

| | | | Coordinates |
|---|---|---|---|
| Homology | 5′ primer | 3′ primer | (GRCh38 |
| Arm | (SEQ ID NO) | (SEQ ID NO) | assembly) |
| 5′; 333 bp | ACTACGATTCCATCAATGCCT | CTTATCGATGATAAGCTGTCA | On the |
| | (SEQ ID NO: 39) | (SEQ ID NO: 40) | backbone |
| | | | of pBACe3.6 |
| 3′; 213 bp | CCACCTCAGCCTGGATTTCA | TACATTGGTGCTGGGAGTGG | chr14: |
| | (SEQ ID NO: 41) | (SEQ ID NO: 42) | 21548298- |
| | | | 21548510 |

The final MAID 6980 construct contained from 5′ to 3′: 5′ mouse homology arm (GRCm38 coordinates: chr14:52411629-52426985), the same mouse arm previously used in MAID1771, a Hygromycin-resistance cassette flanked by Loxp, the −74 kb human genomic sequence including a human CTCF binding element (GRCh38 coordinates: chr14:21548298-21618860), a 90 kb of 3′ human homology arm of human TCRA sequence (GRCh38 coordinates: chr14:21618860-21709339), and a chloramphenicol-resistance cassette. Final clone was selected based on CM/Hygromycin resistance.

MAID6980 targeting vector was electroporated into mouse embryonic stem (ES) cells comprising a humanized TRA and TRD locus, which is depicted as MAID6317 with the selection cassettes deleted in intermediate steps. Targeted homologous recombination resulted in insertion of 74 kb human sequence with CTCF binding element (GRCh38 coordinates chr14:21548298-21618860) at 5′end of human TRV1-1 gene. Successful integration was confirmed by a modification of allele (MOA) assay as described, e.g., in Valenzuela et al, supra. GOA (gain of allele) probes used for the MOA assay for detecting presence of 5′ end of human TCRA sequences are depicted in Table 6 below, together with their approximate locations. The selection cassette was subsequently removed by transient expression of CRE recombinase in the ES cell clone, to be MAID6981. (See FIG. 4).

TABLE 6

| Probe | | Coordinates (GRCh38 |
|---|---|---|
| Name | Probe (5′ to 3′) | assembly) |
| 785h1 | TTCAAACCTCACTCGGAGTTCTTGGG | chr14:21569802-21569827 |
| | (SEQ ID NO: 13) | |
| 785h2 | TTCTTCACAGCTTGTCTCTGGGC | chr14:21557481-21557503 |
| | (SEQ ID NO: 14) | |
| 785h3 | AGGGCTGCCATGTATCCTACAGGA | chr14:21582319-21582342 |
| | (SEQ ID NO: 15) | |

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein by reference). The mouse embryo comprising the donor ES cells was incubated in vitro and then implanted into a surrogate mother to produce an FO mouse fully derived from the donor ES cells. Mice bearing a human TCRD genes were identified by genotyping using the MOA assay described above. Mice heterozygous for the human TCRD genes (and TCRA genes) were bred to homozygosity.

### Example 1.2: Genetic Engineering of Mice with Human TCR Gamma Locus

Human TCRG locus genes were introduced into the mouse genome using VELOCIGENE® technology (see, e.g., U.S. Pat. No. 6,586,251 and Valenzuela et al. (2003) High-throughput engineering of the mouse genome coupled with high-resolution expression analysis. Nat. Biotech. 21(6): 652-659, both incorporated herein by reference). A 153 kb of genomic sequence comprising human TCRG, from hTRGV1 to hTRGC2 (coordinates chr7:38383439-38230960 (GRCh38 assembly), starting from upstream of a human binding element, replaced a 179 kb sequence comprising mouse Tcrg locus on mouse chromosome 13 (coordinates chr13:19177876-19357359 (GRCm38 assembly)), by deletion of mouse Tcrg locus and subsequent insertion of human TCRG locus, FIGS. 3A and 3B.

In detail, for deletion of 179 kb of mouse Tcrg locus genes from mTrgv7 to mTrgc4, a loxp-Neo cassette added into mouse BAC clone RP23-212n5 by bacterial homologous recombination (BHR). Mouse homology arms were made by PCR amplification using BAC clone RP23-212n5 as the template, and are indicated in Table 7 below.

TABLE 7

| | | | Coordinates |
|---|---|---|---|
| Homology | | | (GRCm38 |
| Arm | 5′ primer | 3′ primer | assembly) |
| 5′; 136 bp | CCAGTTTCTCCCCACTTGCT | AACCAGCTGTCCAATGGGAG | chr13:19177740- |
| | (SEQ ID NO: 16) | (SEQ ID NO: 17) | 19177875 |
| 3′; 151 bp | TGATGCAGCCTCACAGTCTG | AAAGGCTGTGGCTGCTTTTG | chr13:19357360- |
| | (SEQ ID NO: 18) | (SEQ ID NO: 19) | 19357510 |

In this targeting vector (designated MAID20143 (VI845)), the mouse homology arms were assembled into a construct containing, from 5′ to 3′: a chloramphenicol-resistance cassette, the 5′ mouse arm, an ICeu1 site, a Neomycin-resistance cassette flanked by two loxp sites, a Mre1 site and the 3′ mouse arm. See FIG. 3A.

MAID20143 (VI845) targeting vector was electroporated into mouse embryonic stem (ES) cells. Targeted homologous recombination resulted in deletion of 179 kb of mouse sequence (GRCm38 coordinates: chr13:19177876-19357359) of mouse Tcrg locus (See FIG. 3A; MAID20143). Successful integration was confirmed by a modification of allele (MOA) assay as described, e.g., in Valenzuela et al, supra. Probes used for the MOA assay for detecting absence of mouse TCRG sequences (loss of allele) are depicted in Table 8 below, and coordinates according to mouse genome assembly GRCm38 are also included.

TABLE 8

| | | Coordinates |
|---|---|---|
| Probe | | (GRCm38 |
| Name | Probe (5′ to 3′) | assembly) |
| TCRg mTF | AAATTTGTGCTTCGGAATGTGGAGGA | chr13:19178428- |
| | (SEQ ID NO: 20) | 19178453 |
| TCRg mTN | CATCGCTTTATGGATGTCTCTG (SEQ ID | chr13:19237089- |
| | NO: 21) | 19237110 |
| TCRg mTV | TCAGAAGAGCAGCCATGTCCTGT (SEQ ID | chr13:19352303- |
| | NO: 22) | 19352325 |

For the humanization TCRG, the human insert containing the TCRG locus genes and flanking sequences was made by two sequential bacterial homologous recombination (BHR) modifications of BAC clone CTD-2563o9. Human homology arms were made by PCR amplification using human BAC clone CTD-2563o9 as the template and are indicated in Table 9 below.

TABLE 9

| | | | Coordinates |
|---|---|---|---|
| Homology | | | (GRCh38 |
| Arm | 5′ primer | 3′ primer | assembly) |
| 5′; 152 bp | CCCTGGGCCCACTATTGTTT | AGTGGTGATGATGCTGGCTT | chr7:38383439- |
| | (SEQ ID NO: 23) | (SEQ ID NO: 24) | 38383590 |
| 3′; 175 bp | CCCTGGGCCCACTATTGTTT | AGTGGTGATGATGCTGGCTT | chr7:38230786- |
| | (SEQ ID NO: 25) | (SEQ ID NO: 26) | 38230960 |

In the first BHR step, −18 kb sequence was deleted from the 5′ end of the human BAC, and replaced with a hygromycin-resistance cassette flanked by loxp sites (loxp-hyg-loxp) with a 5′ Iceu1 site to make construct VI855. (See FIG. 3B). In the second BHR step, a spectinomycin (Spec) resistance cassette with a 3′ Mre1 site replaced 23 kb at the 3′ end of the human BAC to make the construct VI893, which now contained from 5′ to 3′: an I-CeuI site, a loxp-hyg-loxp cassette, 153 kb of genomic sequence comprising human TCRG, a Mre1 site, and a Spec cassette. The human TCRG sequence included all human TCRG segments and genes from hTRGV1 to hTRGC2 (GRCh38 coordinates chr7:38383439-38230960).

To make the final targeting vector (designated MAID20200 (VI902)), constructs VI893 and VI845 were both digested with I-CeuI and Mre1 restriction enzymes and ligated together. See, e.g., FIG. 3B. The final targeting vector contained, from 5′ to 3′: a chloramphenicol-resistance cassette, the 5′ mouse homology arm (containing mouse CTCF binding element), an I-CeuI site, the loxp-hyg-loxp cassette, the 153 kb human genomic insert comprising the human TCRG sequence comprising all human TCRG segments and genes from hTRGV1 to hTRGC2, an mre1 site and the 3′ mouse homology arm; and final clone was selected based on CM/hygromycin resistance. Although not depicted in FIG. 3B, VI902 comprises all 15 hTRGV segments, all 5 hTRGJ segments, and both hTRGC genes in germline configuration.

MAID20200 targeting vector was electroporated into mouse embryonic stem (ES) cells MAID20143. Targeted homologous recombination resulted in insertion of −153 kb of the human genomic insert comprising the human TCRG sequence comprising all human TCRG segments and genes from hTRGV1 to hTRGC2. Successful integration was confirmed by a modification of allele (MOA) assay as described, e.g., in Valenzuela et al, supra. Probes used for the MOA assay (gain of allele) for detecting presence of human TCRG sequence comprising all human TCRG segments and genes from hTRGV1 to hTRGC2 and the loss of mouse Tcrg sequences are depicted in Table 10 below, and coordinates according to mouse genome assembly GRCm38 (loss of allele) or human genome assembly GRCh38 (gain of allele) are also included. The selection cassette was subsequently removed by transient expression of CRE recombinase in the ES cell clone (MAID20201). (See FIG. 4; although not depicted in FIG. 4, MAID20201 comprises all 15 hTRGV segments, all 5 hTRGJ segments, and both hTRGC genes in germline configuration similar to MAID20200).

TABLE 10

| | | Gain of | |
|---|---|---|---|
| | | allele | |
| | | (GOA) | Coordinates |
| | | or loss | (GRCm38 or |
| Probe | | of allele | GRCh38 |
| Name | Probe (5′ to 3′) | (LOA) | assembly) |
| TCRg | AAATCCGCAGCTCGACGCAGCAT | GOA | chr7(human): |
| hTN | (SEQ ID NO: 27) | | 38299931-38299953 |
| TCRg | TCACCAGCCGAGGTACTGGG | GOA | chr7(human): |
| hTV | (SEQ ID NO: 28) | | 38233314-38233333 |
| TCRg | AAATTTGTGCTTCGGAATGTGGAGGA | LOA | chr13(mouse): |
| mTF | (SEQ ID NO: 29) | | 19178428-19178453 |
| TCRg | CATCGCTTTATGGATGTCTCTG | LOA | chr13(mouse): |
| mTN | (SEQ ID NO: 30) | | 19237089-19237110 |
| TCRg | TCAGAAGAGCAGCCATGTCCTGT | LOA | chr13(mouse): |
| mTV | (SEQ ID NO: 31) | | 19352303-19352325 |

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein by reference). The mouse embryo comprising the donor ES cells was incubated in vitro and then implanted into a surrogate mother to produce an FO mouse fully derived from the donor ES cells. Mice bearing a TCRG sequence comprising all human TCRG segments and genes from hTRGV1 to hTRGC2 were identified by genotyping using the MOA assay described above. Mice heterozygous for the human TCRG genes were bred to homozygosity.

### Example 1.3: Generation and Characterization of Mice with Human TCR Delta and TCR Gamma Loci

Mice homozygous for the human TCRD genes (as well as human TCRA genes, as the human TCRD locus is inserted within the humanized TCRA locus), as described in Example 1.1, were bred to mice homozygous for human TCRG genes, as described in Example 1.2. Mice comprising modifications of TCRD (and TCRA) and TCRG loci were bred to homozygosity, and mice homozygous for human TCRD locus, humanized TCRA locus, and human TCRG locus as depicted in FIG. 4 (MAID 6981 and MAID 20201, selection cassettes deleted) were immunophenotyped to determine the presence of TCR delta/gamma positive T cells. Specifically, thymus and spleen tissue from mice with homozygous humanized TCRA, human TCRD, and human TCRG, along with WT mice with fully murine components, were homogenized to single cell suspensions and stained with a mix of flow cytometry antibodies recognizing various surface markers (human γ16 TCR, mouse γδ TCR, mouse TCRβ, mouse CD3c, mouse CD4, mouse CD8α). After staining, cells were washed, fixed in 2% paraformaldehyde, and analyzed on a LSR Fortessa instrument within 48 hours. Mice homozygous for human TCRD locus, humanized TCRA locus, and human TCRG locus expressed γδ T cells in the spleen and thymus (FIGS. 5A and 5B). As in wildtype mice with fully murine components, humanized mice had clearly detectable γ/δ T cells in spleen and thymus that comprised a small fraction (<5%) of T cells overall (FIGS. 5A and 5B). These mice also exhibited normal thymic TCRα/β development and peripheral TCRα/β differentiation as compared to wild type mice (data not shown).

To determine whether mice homozygous for human TCRD locus, humanized TCRA locus, and human TCRG locus also expressed a diversity of productively rearranged human γ/δ T cells, total RNA was isolated from CD90.2⁺ sort-purified spleen and unsorted thymus T-cells using the Rneasy Plus kit (QIAGEN) and reverse-transcribed with SuperScript II (Life Technologies) using a 5′ rapid amplification of cDNA ends primer and oligo(dT) (Table 11). TCRD or TCRG libraries were generated by amplifying cDNA with two rounds of PCR. A 5′ Template Switch (TS) primer paired with primers in mouse Trdc or Trgc2 regions were used to target the amplification. Libraries were indexed with Illumina primers and sequenced on the Illumina MiSeq (2×300 cycles).

TABLE 11

| | | SEQ |
|---|---|---|
| | | ID |
| Primer | Sequence | NO |
| Template switching | 5′-AAGCAGTGGTATCAACGCAGAGTACATrGrGrG-3′ | 32 |
| (TS) primer for | | |
| reverse transcription | | |
| PCR1 forward primer | 5′-AAGCAGTGGTATCAACGCAGAGTACAT-3′ | 33 |
| PCR1 reverse primer | 5′-CTGGGAGAGATGACAATAGCAGGAT-3′ | 34 |
| TCRd constant | | |
| PCR1 reverse primer | 5′-CCCAGAATCGTGTTGCTCTTCTT-3′ | 35 |
| TCRg constant | | |
| PCR2 forward primer | 5′- | 36 |
| | GTGACTGGAGTTCAGACGTGTGCTCTTCCGATCT | |
| | AAGCAGTGGTATCAACGCAGAGT-3′ | |
| PCR2 reverse primer | 5′- | 37 |
| TCRd constant | ACACTCTTTCCCTACACGACGCTCTTCCGATCTAA | |
| | ACGGATGGTTTGGTATGAGGCTG-3′ | |
| PCR2 reverse primer | 5′- | 38 |
| TCRg constant | ACACTCTTTCCCTACACGACGCTCTTCCGATCTGA | |
| | AGGAAGAAAAATAGTGGGCTTGG-3′ | |

Raw sequencing reads were demultiplexed and filtered on the basis of quality, length, and perfect match to corresponding constant region primers. Overlapping paired-end reads were merged and aligned using a locally installed IgBLAST (National Center for Biotechnology Information, v2.2.25+) to human TCR V and J gene databases. CDR3 sequences were extracted using International ImMunoGeneTics Information System boundaries.

As depicted in FIGS. 6A-6B and 7A-7B, TCR repertoire analysis of thymus and spleen mRNA revealed successful diverse V(D)J gene usage, indicating proper recombination, expression and thymic selection of humanized γδ TCRs. Recombination of a diverse repertoire of V(D)J gene segments was also seen in γ/δ T cells in skin and gut mucosa (data not shown). Expression of TCRs with TRAVs or TRAJs with TRD constant regions was also detected, indicating productive rearrangement of some TRAVs or TRAJs into γδ TCRs (FIGS. 6A and 6B). Some TRAVs have previously been reported to rearrange and express as γδ TCRs, but such diverse representation of TRAVs and TRAJs in γδ TCRs has not been previously reported. Additionally, the high percent identity between TRGJ1*02 and TRGJ2*01 did not allow distinguishing between the expression of the two gene segments, and data for TRGJ1 in FIG. 6B likely represents a combination of the expression of both gene segments.

### Example 2: Generation of Mice Comprising Humanized Cellular Immune System Components

In order to generate mice comprising humanized cellular immune system components, mice homozygous for humanization of various components, e.g., TCRγ and TCRδ, TCRα and β (e.g., mice homozygous for humanized TCRβ loci comprising either fully human TCRBDJ1 and TCRBDJ2 clusters or e.g., mice homozygous for humanized TCRβ loci comprising humanized TCRBDJ1 and TCRBDJ2 clusters with murine TCRB noncoding sequences and human coding sequences), MHC I, β2M, MHC II α and β, CD4, and CD8α and CD8β, may be bred together in any combination to create mice that have different components of the T cell immune response humanized. For example, a mouse comprising a humanized MHC I may be bred with a mouse comprising a humanized β2M to generate a mouse expressing humanized MHC I/132M.

Non-limiting exemplary mice comprising chimeric human/mouse TRB loci (e.g., TRB loci comprising humanized TCRBDJ1 and TCRBDJ2 clusters with murine TCRB noncoding sequences and human coding sequences) that may be bred with the mice made in Example 1 are described in U.S. Pat. No. 9,113,616 and Moore, M. et al. Sci Immunol 6 (2021); doi:10.1126/sciimmunol.abj4026; each of which is incorporated herein in its entirety by reference. Non-limiting exemplary mice comprising chimeric human/non-human T cell co-receptor loci (e.g., CD4, CD8α and/or CD8β) that may be bred with the mice made according to Example 1 are described in U.S. Pat. Nos. 9,848,587 and 10,820,581, each of which is incorporated herein by reference. Non-limiting exemplary mice comprising chimeric human/non-human (e.g., human/rodent, e.g., human/mouse) MHC I and/or MHC II genes, and human or humanized 32-microglobulin, that may be bred with the mice made according to Example 1 are described in U.S. Pat. Nos. 8,847,005; 9,043,996; 9,591,835; 9,615,550; and 10,154,658; each of which are incorporated herein by reference.

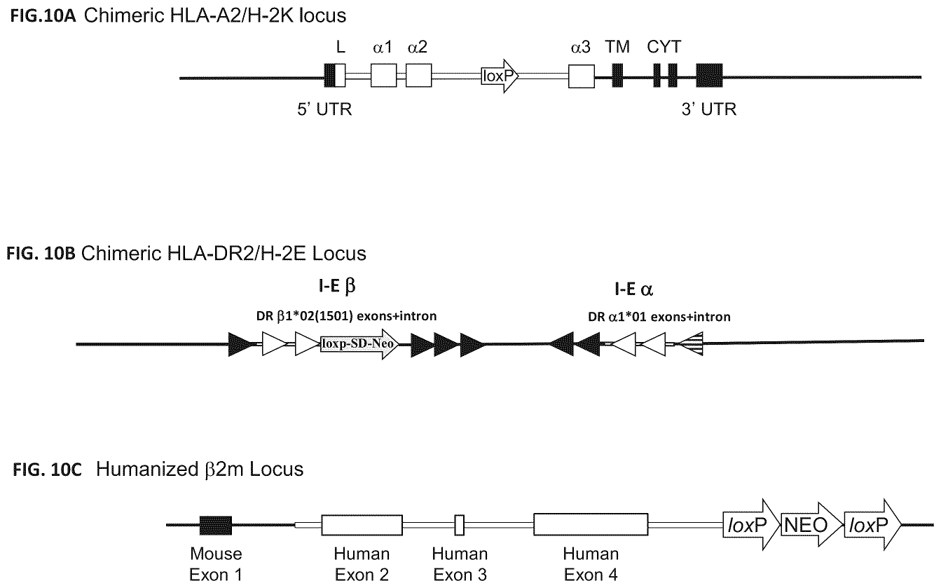

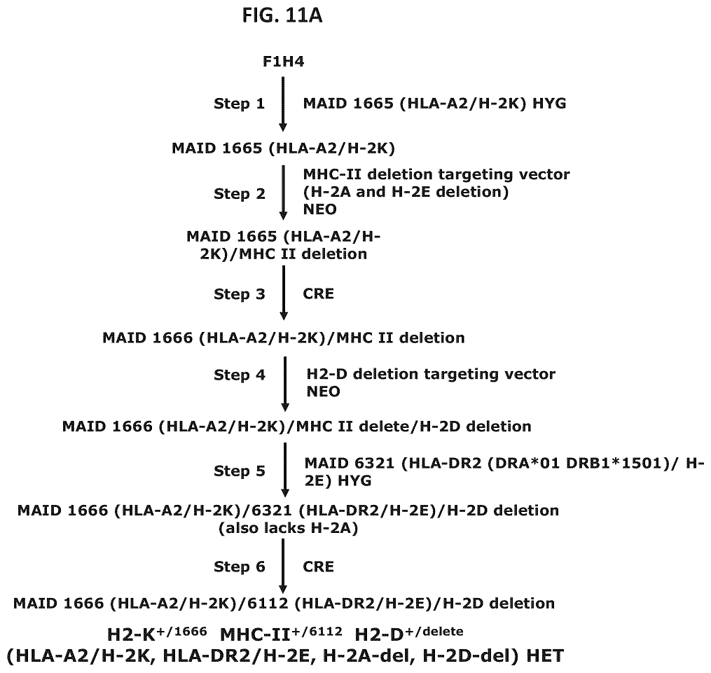

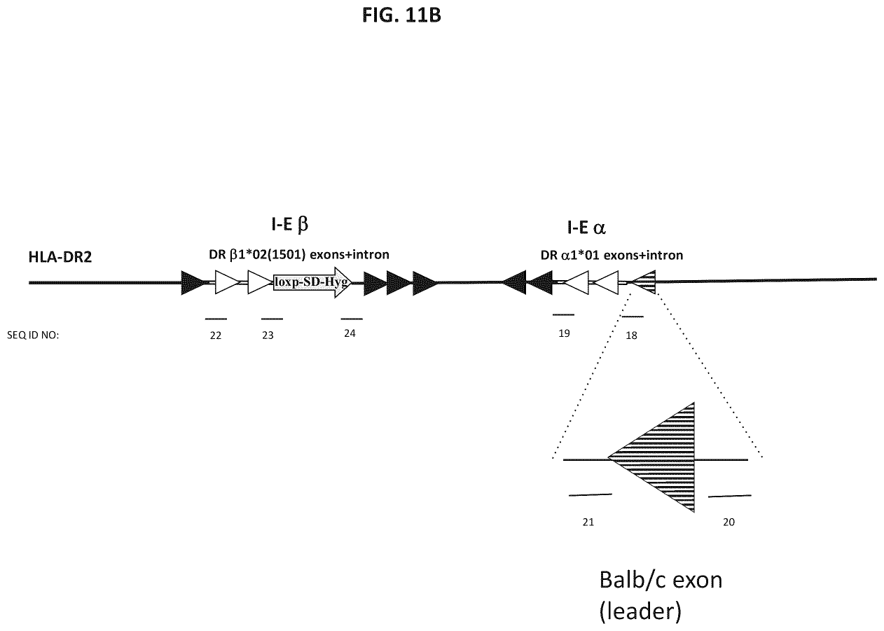

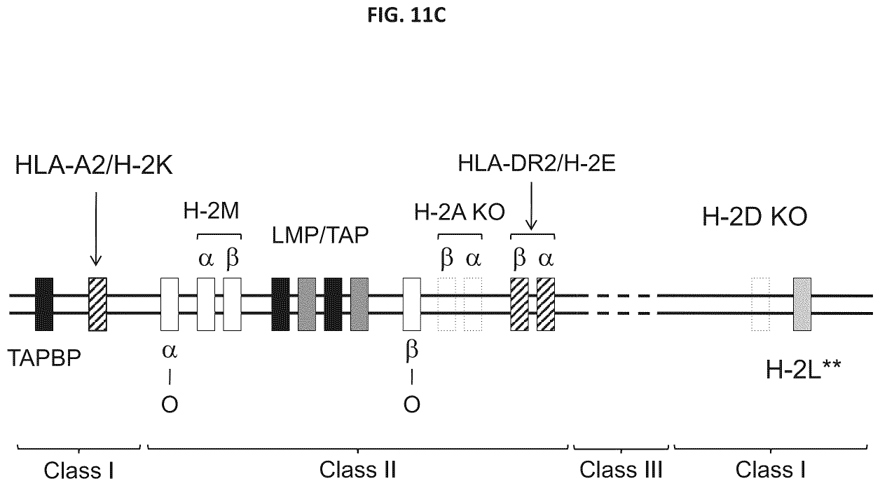

Schematics (not depicted to scale) providing the genetic modifications in mice comprising humanized TCRα and 13 (e.g., mice homozygous for humanized TCRβ loci comprising either fully human TCRBDJ1 and TCRBDJ2 clusters or e.g., mice homozygous for humanized TCRβ loci comprising humanized TCRBDJ1 and TCRBDJ2 clusters with murine TCRB noncoding sequences and human coding sequences), MHC I, β2M, MHC II α and β, CD4, and CD8α and CD8β loci, that are bred or otherwise incorporated together into a single mouse with TCRD/G genetic modification are depicted in FIGS. 8-11, and described in respective patent and non-patent literature cited herein. Mice homozygous for humanization of various components, e.g., MHC I, MHC II α and β, TCRα and β, TCRγ and TCRδ, CD4, CD8α and β, and β2M are bred together using methods known in the art to obtain a mouse comprising all humanizations of all these components of the cellular immune system. Mice are bred to homozygosity using methods known in the art. Alternatively, targeting vectors comprising each humanized gene (e.g., as depicted in FIGS. 8-11) can be introduced via sequential targeting into the same ES cell to obtain an ES cell comprising all nine humanizations, and the resultant ES cell is introduced into 8-cell stage mouse embryo by the VELOCIMOUSE® method, described in Example 1 above.

Thus, VelociTαβγδ mice (e.g., mice homozygous for humanized TCRα and 13 loci, human TCRγ and TCRδ loci, one or more humanized MHC I loci (e.g., HLA-A/H2-K and HLA-B/H2-D), one or more humanized MHC II α and 13 loci (e.g., HLA-DR2/H2-E and HLA-DQ/H2-A) loci, a humanized CD4 locus, humanized CD8α and β loci, and a humanized β2M loci) were obtained. VelociTαβγδ mice were immunophenotyped to determine the presence of T cells expressing human γδ TCR. Specifically, spleen and thymus tissue from VelociTαβγδ mice, along with wildtype (WT) mice with fully murine cellular immune components, were homogenized to single cell suspensions and stained with a mix of flow cytometry antibodies recognizing various surface markers (human γ/δ TCR, mouse γδ TCR, CD3, CD45). After staining, cells were washed, fixed in 2% paraformaldehyde, and analyzed on a LSR Fortessa instrument within 48 hours. Mice homozygous for human TCRD locus, human TCRG locus, humanized TCRA locus, and humanized TCRB locus comprise T cells expressing human γδ TCR in the spleen (FIG. 12A) and thymus FIG. 12B).

To test expression of human γδ by T cells in the gut, cells isolated from mesenteric lymph nodes (MLN), and intraepithelial lymphocytes (IEL) isolated from the small and large intestine were analyzed (Panea et al. (2021) Commun Biol. 4(1):913). As depicted in FIG. 12C, tissue-resident T cells expressing human γδ TCR were detected at the locations in VelociTαβγδ mice in patterns consistent with T cells expressing fully murine γδ TCR in WT mice.

To test expression of human γδ by T cells in the skin, cells isolated from skin epidermal layer using the Epidermis Dissociation Kit manufactured by Miltenyi Biotec (catalog #130-095-928). As depicted in FIG. 12D, skin-resident T cells expressing human γδ TCR were detected at the locations in VelociTαβγδ mice in patterns consistent with T cells expressing fully murine γδ TCR in WT mice. In addition, sequencing data confirmed the diversity of productive TCRD and TCRG V(D)J rearrangements in the spleen and thymus of VelociTαβγδ mice was similar to that seen in mice described in Example 1.3 (data not shown).

### EQUIVALENTS

Those skilled in the art will recognize, or be able to ascertain using no more than routine experimentation, many equivalents of the specific embodiments of the invention described herein. Such equivalents are intended to be encompassed by the following claims.

Entire contents of all non-patent documents, patent applications and patents cited throughout this application are incorporated by reference herein in their entirety.

1. A mouse comprising:
(I) germ cells and CD3⁻ somatic cells that comprise an unrearranged TCR γ variable region sequence comprising an unrearranged human TCR Vγ segment and an unrearranged human TCR Jγ segment,
wherein the unrearranged TCR γ variable region sequence is operably linked to a human TCRγ constant region gene sequence, optionally at an endogenous TCRγ locus,
wherein the unrearranged human TCR Vγ segment and the unrearranged human TCR Jγ segment rearrange in a T cell of the mouse to form a rearranged human TCR Vγ/Jγ variable region gene sequence that is operably linked to the human TCR γ constant region gene sequence, and
wherein the rearranged human TCR Vγ/Jγ variable region gene sequence operably linked to the human TCR γ constant region gene sequence together encode a human TCR γ polypeptide, and
(II) a CD3⁺ T cell that expresses, on its surface, a functional TCR that comprises the human TCR γ polypeptide.

2. The mouse of claim 1, wherein the germ cells and CD3⁻ somatic cells further comprise an unrearranged T cell receptor (TCR) δ variable region sequence comprising an unrearranged human TCR Vδ segment, an unrearranged human TCR Dδ segment, and an unrearranged human TCR Jδ segment,
wherein the unrearranged TCR δ variable region sequence is operably linked to a human TCR δ constant region gene sequence, optionally at an endogenous TCR δ locus,
wherein the unrearranged human TCR Vδ segment, the unrearranged human TCR Dδ segment, and the unrearranged human TCR Jδ segment rearrange in a T cell of the mouse to form a rearranged human TCR Vδ/Dδ/Jδ variable region gene sequence that is operably linked to the human TCR δ constant region gene sequence, and
wherein the rearranged human TCR Vδ/Dδ/Jδ variable region gene sequence operably linked to the human TCR δ constant region gene sequence together encode a human TCR δ polypeptide,
wherein the mouse comprises a CD3⁺ T cell that expresses, on its surface, a functional TCR that comprises both the human TCR γ polypeptide and the human TCR δ polypeptide.

3. The mouse of claim 2, wherein the germ cells and CD3⁻ somatic cells further comprise an unrearranged human TCR Vα segment upstream of the unrearranged TCR δ variable region sequence and the human TCR δ constant region gene sequence,
wherein the unrearranged human TCR Vα segment, the unrearranged human TCR Dδ, and the unrearranged human TCR Jδ segment rearrange in a T cell of the mouse to form a rearranged human TCR Vα/Dδ/Jδ variable region gene sequence that is operably linked to the human TCR δ constant region gene sequence,
wherein the rearranged human TCR Vα/Dδ/Jδ variable region gene sequence operably linked to the human TCR δ constant region gene sequence together encode a human hybrid TCR polypeptide comprising a human hybrid TCR α/δ variable domain and a human TCR δ constant domain, and
wherein the mouse comprises a CD3⁺ T cell that expresses, on its surface, a functional TCR that comprises the human hybrid TCR polypeptide comprising the human hybrid α/δ variable domain and the human TCR δ constant domain.

4. A mouse comprising:
(I) germ cells and CD3⁻ somatic cells that comprise from 5′ to 3:
an unrearranged human TCR Vα segment and
an unrearranged TCR δ variable region sequence comprising an unrearranged human TCR Vδ segment, an unrearranged human TCR Dδ segment, and an unrearranged human TCR Jδ segment,
wherein the unrearranged TCR δ variable region sequence is operably linked to a human TCR δ constant region gene sequence, optionally at an endogenous TCR δ locus,
wherein the unrearranged human TCR Vα segment, the unrearranged human TCR Dδ, and the unrearranged human TCR Jδ segment rearrange in a T cell of the mouse to form a rearranged human TCR Vα/Dδ/Jδ variable region gene sequence that is operably linked to the human TCR δ constant region gene sequence,
wherein the rearranged human TCR Vα/Dδ/Jδ variable region gene sequence operably linked to the human TCR δ constant region gene sequence together encode a human hybrid TCR polypeptide comprising a human hybrid TCR α/δ variable domain and a human TCR δ constant domain,
(II) a CD3⁺ T cell that expresses, on its surface, a functional TCR that comprises the human hybrid TCR comprising the human hybrid α/δ variable domain and the human TCR δ constant domain.

5. The mouse of claim 4, wherein the germ cells and CD3⁻ somatic cells comprise a replacement of an endogenous TCR Vα segment with the unrearranged human TCR Vα segment and a replacement of an endogenous TCR Jα segment with an unrearranged human TCR Jα segment,
wherein the unrearranged human TCR Vα segment and unrearranged human TCR Jα segment are operably linked to each other and a TCR α constant region gene sequence, such as a mouse TCR α constant region gene sequence, and
wherein the unrearranged human TCR Vα segment and unrearranged human TCR Jα segment rearrange in a T cell of the mouse to form a rearranged TCR Vα/Jα variable region gene sequence operably linked to the TCR α constant region gene sequence,
wherein the rearranged human TCR Vα/Jα variable region gene sequence operably linked to the TCR α constant region gene sequence together encode a TCR α polypeptide comprising a human TCR α variable domain and a TCR α constant domain, and
wherein the mouse comprises a CD3⁺ T cell that expresses, on its surface, a functional TCR comprising the TCR α polypeptide.

6. The mouse of claim 4, wherein the germ cells and CD3⁻ somatic cells comprise a replacement of all endogenous TCR Vα segments with a full repertoire of unrearranged human TCR Vα segments and a replacement of all endogenous TCR Jα segments with a full repertoire of unrearranged human TCR Jα segments,
wherein the full repertoire of unrearranged human TCR Vα segments and the full repertoire of unrearranged human TCR Jα segments are operably linked to each other and a mouse TCR α constant region gene sequence at an endogenous TCR α locus, and
wherein the full repertoire of unrearranged human TCR Vα segments and the full repertoire of unrearranged human TCR Jα segments rearrange in a T cell of the mouse to form a rearranged human TCR Vα/Jα variable region gene sequence operably linked to the mouse TCR α constant region gene sequence,
wherein the rearranged TCR Vα/Jα variable region gene sequence operably linked to the mouse TCR α constant region gene sequence together encode a chimeric TCR α polypeptide comprising a human TCR α variable domain operably linked to a mouse TCR α constant domain, and
wherein the mouse comprises a CD3⁺ T cell that expresses, on its surface, a functional TCR that comprises the chimeric TCR α polypeptide.

7. The mouse of claim 1, wherein the germ cells and CD3⁻ somatic cells comprise:
(A) a replacement of an endogenous TCR Vγ segment with an unrearranged human TCR Vγ segment, a replacement of an endogenous TCR Jγ segment with an unrearranged human TCR Jγ segment, and a replacement of an endogenous TCR γ constant region gene sequence with a human TCR γ constant region gene sequence; or
(B) a replacement of an endogenous TCR Vδ segment with an unrearranged human TCR Vδ segment, a replacement of an endogenous TCR Dδ segment with an unrearranged human TCR Dδ segment, a replacement of an endogenous TCR Jδ segment with an unrearranged human TCR Jδ segment, and a replacement of an endogenous TCR δ constant region gene sequence with a human TCR δ constant region gene sequence; or
(C) (i) a replacement of an endogenous TCR Vγ segment with an unrearranged human TCR Vγ segment, a replacement of an endogenous TCR Jγ segment with an unrearranged human TCR Jγ segment, and a replacement of an endogenous TCR γ constant region gene sequence with a human TCR γ constant region gene sequence, and
(ii) a replacement of an endogenous TCR Vδ segment with an unrearranged human TCR Vδ segment, a replacement of an endogenous TCR Dδ segment with an unrearranged human TCR Dδ segment, a replacement of an endogenous TCR Jδ segment with an unrearranged human TCR Jδ segment, and a replacement of an endogenous TCR δ constant region gene sequence with a human TCR δ constant region gene sequence.

8. The mouse of claim 7, wherein:
(A) the unrearranged TCR Vγ segment comprises a full repertoire of unrearranged human TCR Vγ segments and the unrearranged human TCR Jγ segment comprises a full repertoire of unrearranged human TCR Jγ segments;
(B) the unrearranged human TCR Vδ segment comprises a full repertoire of unrearranged human TCR Vδ segments, the unrearranged human TCR Dδ segment comprises a full repertoire of unrearranged human TCR Dδ segments, and the unrearranged human TCR Jδ segment comprises a full repertoire of unrearranged human TCR Jδ segments; or
(C) (i) the unrearranged TCR Vγ segment comprises a full repertoire of unrearranged human TCR Vγ segments and the unrearranged human TCR Jγ segment comprises a full repertoire of unrearranged human TCR Jγ segments, and
(ii) the unrearranged human TCR Vδ segment comprises a full repertoire of unrearranged human TCR Vδ segments, the unrearranged human TCR Dδ segment comprises a full repertoire of unrearranged human TCR Dδ segments, and the unrearranged human TCR Jδ segment comprises a full repertoire of unrearranged human TCR Jδ segments.

9. The mouse of claim 1, wherein:
(I) the germ cells and CD3⁻ T cells comprise:
(A) at the endogenous TCR γ locus:
a replacement of all endogenous TCR Vγ segments with a full repertoire of unrearranged human TCR Vγ segments,
a replacement of all endogenous TCR Jγ segments with a full repertoire of unrearranged human TCR Jγ segments, and
a replacement of all TCR γ constant region gene sequences with a full repertoire of human TCR γ constant region gene sequences; and
(B) at the endogenous TCR δ locus:
a replacement of all endogenous TCR Vδ segments with a full repertoire of unrearranged human TCR Vδ segments,
a replacement of all endogenous TCR Dδ segments with a full repertoire of unrearranged human TCR Dδ segments,
a replacement of all endogenous TCR Jδ segments with a full repertoire of unrearranged human TCR Jδ segments, and
a replacement of an endogenous TCR δ constant region gene sequence with a human TCR δ constant region gene sequence; and
(II) the mouse comprises a CD3⁺ T cell that expresses, on its surface, a functional TCR comprising a human TCR γ polypeptide and a human TCR δ polypeptide.

10. The mouse of claim 5, wherein the germ cells and CD3⁻ somatic cells further comprise an unrearranged TCRβ variable region sequence comprising at least one unrearranged human TCR variable region Vβ segment, at least one unrearranged human TCR variable region Dβ segment, and at least one unrearranged TCR variable region Jβ segment,
wherein the unrearranged TCR β variable region sequence is operably linked to a TCR β constant region gene sequence, such as a mouse TCR β constant region gene sequence, optionally at an endogenous TCR β locus,
wherein the unrearranged human TCR Vβ segment, the unrearranged human TCR Dβ segment, and the unrearranged human TCR Jβ segment rearrange in a T cell of the mouse to form a rearranged human TCR Vβ/Dβ/Jβ variable region gene sequence that is operably linked to the TCR β constant region gene sequence, and
wherein the rearranged human TCR Vβ/Dβ/Jβ variable region gene sequence operably linked to the TCR β constant region gene sequence together encode a TCR β polypeptide comprising a human TCR β variable domain and a TCR β constant domain; and
wherein the mouse comprises a CD3⁺ T cell that expresses, on its surface, a functional TCR comprising the TCR β polypeptide.

11. The mouse of claim 10, wherein the unrearranged TCRβ variable region sequence comprises a mouse TCRB non-coding sequence.

12. The mouse of claim 1, wherein:
(I) the germ cells and CD3⁻ somatic cells comprise:
(A) at the endogenous TCR γ locus:
a replacement of all endogenous TCR Vγ segments with a full repertoire of unrearranged human TCR Vγ segments,
a replacement of all endogenous TCR Jγ segments with a full repertoire of unrearranged human TCR Jγ segments, and
a replacement of all TCR γ constant region gene sequences with a full repertoire of human TCR γ constant region gene sequences; and
(B) at the endogenous TCR δ locus:
a replacement of all endogenous TCR Vδ segments with a full repertoire of unrearranged human TCR Vδ segments,
a replacement of all endogenous TCR Dδ segments with a full repertoire of unrearranged human TCR Dδ segments,
a replacement of all endogenous TCR Jδ segments with a full repertoire of unrearranged human TCR Jδ segments, and
a replacement of an endogenous TCR δ constant region gene sequence with a human TCR δ constant region gene sequence; and
(C) at the endogenous TCR α locus:
a replacement of all endogenous TCR Vα segments with a full repertoire of unrearranged human TCR Vα segments, and
a replacement of all endogenous TCR Jα segments with a full repertoire of unrearranged human TCR Jα segments, and
(D) at the endogenous TCR β locus:
a replacement of all endogenous TCR Vβ segments with a full repertoire of unrearranged human TCR Vβ segments,
a replacement of all endogenous TCR Dβ segments with a full repertoire of unrearranged human TCR Dβ segments, and
a replacement of all endogenous TCR Jβ segments with a full repertoire of unrearranged human TCR Jβ segments; and
(II) the mouse further comprises a CD3⁺ T cell that expresses, on its surface, a functional human TCR comprising a human TCR γ polypeptide and a human TCR δ polypeptide and a CD3⁺ T cell that expresses, on its surface, a functional human or humanized TCR comprising a human or humanized TCR α polypeptide and a human or humanized TCR β polypeptide.

13. The mouse of claim 1, wherein:
(I) the germ cells and CD3⁻ somatic cells comprise:
(A) at the endogenous TCR γ locus:
a replacement of all endogenous TCR Vγ segments with a full repertoire of unrearranged human TCR Vγ segments,
a replacement of all endogenous TCR Jγ segments with a full repertoire of unrearranged human TCR Jγ segments, and
a replacement of all TCR γ constant region gene sequences with a full repertoire of human TCR γ constant region gene sequences; and
(B) at the endogenous TCR δ locus:
a replacement of all endogenous TCR Vδ segments with a full repertoire of unrearranged human TCR Vδ segments,
a replacement of all endogenous TCR Dδ segments with a full repertoire of unrearranged human TCR Dδ segments,
a replacement of all endogenous TCR Jδ segments with a full repertoire of unrearranged human TCR Jδ segments, and
a replacement of an endogenous TCR δ constant region gene sequence with a human TCR δ constant region gene sequence; and
(C) at the endogenous TCR α locus:
a replacement of all endogenous TCR Vα segments with a full repertoire of unrearranged human TCR Vα segments, and
a replacement of all endogenous TCR Jα segments with a full repertoire of unrearranged human TCR Jα segments;
(D) at the endogenous TCR β locus:
a replacement of all endogenous TCR Vβ segments with a full repertoire of unrearranged human TCR Vβ segments,
a replacement of all endogenous TCR Dβ segments with a full repertoire of unrearranged human TCR Dβ segments,
a replacement of all endogenous TCR Jβ segments with a full repertoire of unrearranged human TCR Jβ segments; and
(E) a first nucleotide sequence encoding a chimeric human/mouse CD4 co-receptor that comprises D1, D2 and D3 domains of a human CD4 polypeptide operably linked to D4, transmembrane and cytoplasmic domains of a mouse CD4 polypeptide; and
(F) a second nucleotide sequence encoding a chimeric human/mouse CD8α polypeptide and a third nucleotide sequence encoding a chimeric human/mouse CD8β polypeptide,
wherein the chimeric human/mouse CD8α polypeptide comprises an IgV-like domain of a human CD8α polypeptide operably linked to transmembrane and cytoplasmic domains of a mouse CD8α polypeptide and wherein the chimeric human/mouse CD8β polypeptide comprises an IgV-like domain of a human CD8β polypeptide operably linked to transmembrane and cytoplasmic domains of a mouse CD8β polypeptide; and
(G) a first nucleic acid sequence encoding a chimeric human/mouse MHC II α polypeptide and a second nucleic acid sequence encoding a chimeric human/mouse MHC II β polypeptide,
wherein the chimeric human/mouse MHC II α polypeptide comprises α1 and α2 domains of a human HLA class II α polypeptide operably linked to transmembrane and cytoplasmic domains of a mouse MHC II α polypeptide and wherein the chimeric human/mouse MHC II β polypeptide comprises β1 and β2 domains of a human HLA class II β polypeptide operably linked to transmembrane and cytoplasmic domains of a mouse MHC II β polypeptide;
(H) a third nucleic acid sequence encoding a chimeric human/mouse MHC I polypeptide comprising α1, α2, and α3 domains of a human HLA class I polypeptide operably linked to transmembrane and cytoplasmic domains of a mouse MHC class I polypeptide; and
(I) a polynucleotide encoding a human or humanized β2 microglobulin polypeptide and comprising a nucleotide sequence comprising the nucleotide sequence set forth in exon 1 of a mouse β2 microglobulin gene operably linked to the nucleotide sequence set forth in exon 2, exon 3, and exon 4 of a human β2 microglobulin gene,
(II) wherein the mouse further comprises a CD3⁺ T cell that expresses, on its surface, a functional human TCR comprising a human TCR γ polypeptide and a human TCR δ polypeptide and a CD3⁺ T cell that expresses, on its surface, a functional human or humanized TCR comprising a human or humanized TCR α polypeptide and a human or humanized TCR β polypeptide, and optionally
wherein the mouse expresses:
(e) the chimeric human/mouse CD4 co-receptor,
(f) a chimeric CD8 co-receptor comprising the chimeric human/mouse CD8α polypeptide and the chimeric human/mouse CD8β polypeptide,
(h) a chimeric MHC II complex comprising the chimeric human/mouse MHC II α polypeptide and the chimeric human/mouse MHC polypeptide, wherein the chimeric MHC II complex is capable of binding the chimeric human/mouse CD4 co-receptor,
(i) the chimeric human/mouse MHC I polypeptide, wherein the chimeric MHC I polypeptide is capable of binding the chimeric CD8 co-receptor, and
(j) the human or humanized β2 microglobulin polypeptide.

14.-15. (canceled)

16. The mouse of claim 2, wherein the mouse comprises:
(a) γ/δ T cells in its thymus, its spleen, its skin, and/or its gut mucosa and/or
(b) a population of CD45+CD3+ T cells expressing a human γδ TCR in its thymus, spleen, mesenteric lymph nodes, skin, gut mucosa, and/or among intraepithelial lymphocytes in its colon and/or small intestine, optionally wherein a percentage of the population of CD45+CD3+ T cells expressing a human γδ TCR in its thymus, spleen, mesenteric lymph nodes, skin, gut mucosa, and/or among intraepithelial lymphocytes in its colon and/or small intestine is comparable to or greater than a percentage of the population of CD45+CD3+ T cells expressing a murine γδ TCR in a thymus, spleen, mesenteric lymph nodes, skin, gut mucosa, and/or among intraepithelial lymphocytes in a colon and/or small intestine of a wildtype mouse.

17.-20. (canceled)

21. A mouse embryonic stem (ES) cell or germ cell comprising an unrearranged TCR γ variable region sequence comprising an unrearranged human TCR Vγ segment and an unrearranged human TCR Jγ segment,
wherein the unrearranged TCR γ variable region sequence is operably linked to a human TCRγ constant region gene sequence, optionally at an endogenous TCRγ locus.

22. The mouse ES cell or germ cell of claim 21, wherein the ES cell or germ cell further comprises an unrearranged T cell receptor (TCR) δ variable region sequence comprising an unrearranged human TCR Vδ segment, an unrearranged human TCR Dδ segment, and an unrearranged human TCR Jδ segment,
wherein the unrearranged TCR δ variable region sequence is operably linked to a human TCR δ constant region gene sequence, optionally at an endogenous TCR δ locus.

23. The mouse ES cell or germ cell of claim 22, wherein the ES cell or germ cell further comprises an unrearranged human TCR Vα segment upstream of the unrearranged TCR δ variable region sequence and the human TCR δ constant region gene sequence.

24. A mouse ES cell or germ cell comprising from 5′ to 3′:
an unrearranged human TCR Vα segment and
an unrearranged TCR δ variable region sequence comprising an unrearranged human TCR Vδ segment, an unrearranged TCR Dδ, and an unrearranged human TCR Jδ segment,
wherein the unrearranged TCR δ variable region sequence is operably linked to a human TCR δ constant region gene sequence, optionally at an endogenous TCR δ locus.

25.-37. (canceled)

38. A targeting vector comprising from 5′ to 3′:
(a) a 5′ mouse homology arm,
(b) a human nucleotide sequence set forth at chr7:38383439-38230960 (GRCh38 coordinates), and
(c) a 3′ mouse homology arm.

39. A targeting vector comprising (i) a selection cassette and (ii) a human nucleotide sequence set forth at Chr14:22421820-22464666 (GRCh38 coordinates).

40. A method for generating a nucleic acid sequence encoding a human T cell receptor (TCR) γ variable domain, the method comprising:
immunizing a genetically modified mouse according to claim 1 with an antigen of interest,
allowing said mouse to mount an immune response to the antigen of interest, and
obtaining therefrom a nucleic acid sequence encoding a human TCR γ variable domain that binds the antigen of interest.

41. A method for generating a nucleic acid sequence encoding a human TCR γ polypeptide, the method comprising:
immunizing a genetically modified mouse according to claim 1 with an antigen of interest,
allowing said mouse to mount an immune response to the antigen of interest,
obtaining therefrom a nucleic acid sequence encoding a human TCR γ polypeptide of a TCR that binds the antigen of interest.

42. A method for generating a nucleic acid sequence encoding a human T cell receptor (TCR) δ variable domain, the method comprising:
immunizing a genetically modified mouse according to claim 2 with an antigen of interest,
allowing said mouse to mount an immune response to the antigen of interest, and
obtaining therefrom a nucleic acid sequence encoding a human TCR δ variable domain that binds the antigen of interest.

43. A method for generating a nucleic acid sequence encoding a human TCR δ polypeptide, the method comprising:
immunizing a genetically modified mouse according to claim 2 with an antigen of interest,
allowing said rodent to mount an immune response to the antigen of interest,
obtaining therefrom a nucleic acid sequence encoding a human TCR δ polypeptide of a TCR that binds the antigen of interest.

44. A method for generating a nucleic acid sequence encoding a human hybrid T cell receptor (TCR) α/δ variable domain, the method comprising:
immunizing a genetically modified mouse according to claim 4 with an antigen of interest,
allowing said mouse to mount an immune response to the antigen of interest, and
obtaining therefrom a nucleic acid sequence encoding a human hybrid TCR α/δ variable domain that binds the antigen of interest.

45. A method for making a human therapeutic, the method comprising:
immunizing a genetically modified mouse according to claim 1 with an antigen of interest,
allowing the mouse to mount an immune response,
obtaining, from the mouse, a T cell reactive to the antigen of interest from the mouse,
obtaining, from the T cell, a T cell receptor that binds the antigen of interest and/or a nucleic acid sequence(s) encoding the T cell receptor, wherein the T cell receptor comprises a human TCR variable domain.

46.-51. (canceled)

52. A host cell comprising a nucleic acid molecule made according to claim 40.

53. A method of making a genetically modified mouse or mouse ES cell, comprising modifying the genome of the mouse or mouse ES cell to comprise:
(a) an unrearranged TCR γ variable region sequence comprising an unrearranged human TCR Vγ segment and an unrearranged human TCR Jγ segment,
wherein the unrearranged TCR γ variable region sequence is operably linked to a human TCRγ constant region gene sequence,
wherein the unrearranged human TCR Vγ segment and the unrearranged human TCR Jγ segment rearrange in a T cell of the mouse to form a rearranged human TCR Vγ/Jγ variable region gene sequence that is operably linked to the human TCR γ constant region gene sequence, and
wherein the rearranged human TCR Vγ/Jγ variable region gene sequence operably linked to the human TCR γ constant region gene sequence together encode a human TCR γ polypeptide; and/or
(b) an unrearranged T cell receptor (TCR) δ variable region sequence comprising an unrearranged human TCR Vδ segment, an unrearranged human TCR Dδ segment, and an unrearranged human TCR Jδ segment,
wherein the unrearranged TCR δ variable region sequence is operably linked to a human TCR δ constant region gene sequence,
wherein the unrearranged human TCR Vδ segment, the unrearranged human TCR Dδ segment, and the unrearranged human TCR Jδ rearrange in a T cell of the mouse to form a rearranged human TCR Vδ/Dδ/Jδ variable region gene sequence that is operably linked to the human TCR δ constant region gene sequence, and
wherein the rearranged human TCR Vδ/Dδ/Jδ variable region gene sequence operably linked to the human TCR δ constant region gene sequence together encode a human TCR δ polypeptide.

54. The method of 53, wherein modifying comprises:
(a) replacing an endogenous genomic sequence comprising endogenous TCR Vγ and Jγ gene segments and an endogenous TCR Cγ gene with a heterologous sequence comprising the unrearranged human TCR Vγ segment and the unrearranged human TCR Jγ segment operably linked with the human TCR γ constant region gene sequence and/or
(b) replacing an endogenous genomic sequence comprising endogenous TCR Vδ, Dδ, Jδ gene segments and an endogenous TCR Cδ gene with a heterologous sequence comprising the unrearranged human TCR Vδ segment, the unrearranged human TCR Dδ segment, the unrearranged human TCR Jδ segment and the human TCR δ constant region gene sequence.

55. The method of claim 54, wherein:
(a) the endogenous genomic sequence comprising endogenous TCR Vγ and Jγ gene segments and an endogenous TCR Cγ gene comprises the full repertoire of endogenous TCR Vγ and Jγ gene segments and endogenous TCR Cγ genes, and/or
(b) the endogenous genomic sequence comprising endogenous TCR Vδ, Dδ, Jδ gene segments and an endogenous TCR Cδ gene comprises all endogenous TCR Vδ, Dδ, Jδ gene segments and TCR Cδ genes located between TCR Vα and TCR Jα gene segments.

56. The method of claim 54, or claim 55, wherein:
(a) the heterologous sequence comprising the unrearranged human TCR Vγ segment and the unrearranged human TCR Jγ segment operably linked with the human TCR γ constant region gene sequence comprises a full repertoire of unrearranged TCR Vγ and unrearranged human TCR Jγ segments and all human TCR Cγ genes, and/or
(b) the heterologous sequence comprising the unrearranged human TCR Vδ segment, the unrearranged human TCR Dδ segment, the unrearranged human TCR Jδ segment and the human TCR δ constant region gene sequence comprises a full repertoire of unrearranged TCR Vδ, unrearranged TCR Dδ, and unrearranged human TCR Jδ segments and a human TCR Cδ gene sequence located between a human TCR Vα gene segment and a human TCR Jα gene segment on chromosome 14 of a human genome.

57.-59. (canceled)


SEQUENCE LISTING (42 sequences)

SEQ ID NO: 1 - protein, 1374 aa, synthetic construct
METCYSARGALAILESERLEUARGARGLEULEULEULEULEULEUGLNLEUSERGLNLEULEUALAVALTHRGLNGLYLYSLYSVALVALLEUGLYLYSLYSGLYASPTHRVALGLULEUTHRCYSTHRALASERGLNLYSLYSSERILEGLNPHEHISTRPLYSASNSERASNGLNILELYSILELEUGLYASNGLNGLYSERPHELEUTHRLYSGLYPROSERLYSLEUASNASPARGALAASPSERARGARGSERLEUTRPASPGLNGLYASNPHEPROLEUILEILELYSASNLEULYSILEGLUASPSERASPTHRTYRILECYSGLUVALGLUASPGLNLYSGLUGLUVALGLNLEULEUVALPHEGLYLEUTHRALAASNSERASPTHRHISLEULEUGLNGLYGLNSERLEUTHRLEUTHRLEUGLUSERPROPROGLYSERSERPROSERVALGLNCYSARGSERPROARGGLYLYSASNILEGLNGLYGLYLYSTHRLEUSERVALSERGLNLEUGLULEUGLNASPSERGLYTHRTRPTHRCYSTHRVALLEUGLNASNGLNLYSLYSVALGLUPHELYSILEASPILEVALVALLEUALAPHEGLNLYSALASERSERILEVALTYRLYSLYSGLUGLYGLUGLNVALGLUPHESERPHEPROLEUALAPHETHRVALGLULYSLEUTHRGLYSERGLYGLULEUTRPTRPGLNALAGLUARGALASERSERSERLYSSERTRPILETHRPHEASPLEULYSASNLYSGLUVALSERVALLYSARGVALTHRGLNASPPROLYSLEUGLNMETGLYLYSLYSLEUPROLEUHISLEUTHRLEUPROGLNALALEUPROGLNTYRALAGLYSERGLYASNLEUTHRLEUALALEUGLUALALYSTHRGLYLYSLEUHISGLNGLUVALASNLEUVALVALMETARGVALALAGLNLEUASNASNTHRLEUTHRCYSGLUVALMETGLYPROTHRSERPROLYSMETARGLEUTHRLEULYSGLNGLUASNGLNGLUALAARGVALSERGLUGLUGLNLYSVALVALGLNVALVALALAPROGLUTHRGLYLEUTRPGLNCYSLEULEUSERGLUGLYASPLYSVALLYSMETASPSERARGILEGLNVALLEUSERARGGLYVALASNGLNTHRVALPHELEUALACYSVALLEUGLYGLYSERPHEGLYPHELEUGLYPHELEUGLYLEUCYSILELEUCYSCYSVALARGCYSARGHISGLNGLNARGGLNALAALAARGMETSERGLNILELYSARGLEULEUSERGLULYSLYSTHRCYSGLNCYSPROHISARGMETGLNLYSSERHISASNLEUILE

SEQ ID NO: 2 - protein, 879 aa, Homo sapiens
LYSLYSVALVALLEUGLYLYSLYSGLYASPTHRVALGLULEUTHRCYSTHRALASERGLNLYSLYSSERILEGLNPHEHISTRPLYSASNSERASNGLNILELYSILELEUGLYASNGLNGLYSERPHELEUTHRLYSGLYPROSERLYSLEUASNASPARGALAASPSERARGARGSERLEUTRPASPGLNGLYASNPHEPROLEUILEILELYSASNLEULYSILEGLUASPSERASPTHRTYRILECYSGLUVALGLUASPGLNLYSGLUGLUVALGLNLEULEUVALPHEGLYLEUTHRALAASNSERASPTHRHISLEULEUGLNGLYGLNSERLEUTHRLEUTHRLEUGLUSERPROPROGLYSERSERPROSERVALGLNCYSARGSERPROARGGLYLYSASNILEGLNGLYGLYLYSTHRLEUSERVALSERGLNLEUGLULEUGLNASPSERGLYTHRTRPTHRCYSTHRVALLEUGLNASNGLNLYSLYSVALGLUPHELYSILEASPILEVALVALLEUALAPHEGLNLYSALASERSERILEVALTYRLYSLYSGLUGLYGLUGLNVALGLUPHESERPHEPROLEUALAPHETHRVALGLULYSLEUTHRGLYSERGLYGLULEUTRPTRPGLNALAGLUARGALASERSERSERLYSSERTRPILETHRPHEASPLEULYSASNLYSGLUVALSERVALLYSARGVALTHRGLNASPPROLYSLEUGLNMETGLYLYSLYSLEUPROLEUHISLEUTHRLEUPROGLNALALEUPROGLNTYRALAGLYSERGLYASNLEUTHRLEUALALEUGLUALALYSTHRGLYLYSLEUHISGLNGLUVALASNLEUVALVALMETARG

SEQ ID NO: 3 - protein, 729 aa, synthetic construct
METALASERPROLEUTHRARGPHELEUSERLEUASNLEULEULEULEUGLYGLUSERILEILELEUGLYSERGLYGLUALAARGPROSERGLNPHEARGVALSERPROLEUASPARGTHRTRPASNLEUGLYGLUTHRVALGLULEULYSCYSGLNVALLEULEUSERASNPROTHRSERGLYCYSSERTRPLEUPHEGLNPROARGGLYALAALAALASERPROTHRPHELEULEUTYRLEUSERGLNASNLYSPROLYSALAALAGLUGLYLEUASPTHRGLNARGPHESERGLYLYSARGLEUGLYASPTHRPHEVALLEUTHRLEUSERASPPHEARGARGGLUASNGLUGLYTYRTYRPHECYSSERALALEUSERASNSERILEMETTYRPHESERHISPHEVALPROVALPHELEUPROALALYSPROTHRTHRTHRPROALAPROARGPROPROTHRPROALAPROTHRILEALASERGLNPROLEUSERLEUARGPROGLUALACYSARGPROALAALAGLYGLYALAVALLYSGLYTHRGLYLEUASPPHEALACYSASPILETYRILETRPALAPROLEUALAGLYILECYSVALALALEULEULEUSERLEUILEILETHRLEUILECYSTYRHISARGSERARGLYSARGVALCYSLYSCYSPROARGPROLEUVALARGGLNGLUGLYLYSPROARGPROSERGLULYSILEVAL

SEQ ID NO: 4 - protein, 456 aa, Homo sapiens
ARGPROSERGLNPHEARGVALSERPROLEUASPARGTHRTRPASNLEUGLYGLUTHRVALGLULEULYSCYSGLNVALLEULEUSERASNPROTHRSERGLYCYSSERTRPLEUPHEGLNPROARGGLYALAALAALASERPROTHRPHELEULEUTYRLEUSERGLNASNLYSPROLYSALAALAGLUGLYLEUASPTHRGLNARGPHESERGLYLYSARGLEUGLYASPTHRPHEVALLEUTHRLEUSERASPPHEARGARGGLUASNGLUGLYTYRTYRPHECYSSERALALEUSERASNSERILEMETTYRPHESERHISPHEVALPROVALPHELEUPROALALYSPROTHRTHRTHRPROALAPROARGPROPROTHRPROALAPROTHRILEALASERGLNPROLEUSERLEUARGPROGLUALACYSARGPROALAALAGLYGLYALA

SEQ ID NO: 5 - protein, 630 aa, synthetic construct
METGLNPROTRPLEUTRPLEUVALPHESERMETLYSLEUALAVALLEUHISGLYASNSERVALLEUGLNGLNTHRPROALATYRILELYSVALGLNTHRASNLYSMETVALMETLEUSERCYSGLUALALYSILESERLEUSERASNMETARGILETYRTRPLEUARGGLNARGGLNALAPROSERSERASPSERHISHISGLUPHELEUALALEUTRPASPSERALALYSGLYTHRILEHISGLYGLUGLUVALGLUGLNGLULYSILEALAVALPHEARGASPALASERARGPHEILELEUASNLEUTHRSERVALLYSPROGLUASPSERGLYILETYRPHECYSMETILEVALGLYSERPROGLULEUTHRPHEGLYLYSGLYTHRGLNLEUSERVALVALASPPHELEUPROTHRTHRALAGLNPROTHRLYSLYSSERTHRLEULYSLYSARGVALCYSARGLEUPROARGPROGLUTHRGLNLYSGLYLEUTHRCYSSERLEUTHRTHRLEUSERLEULEUVALVALCYSILELEULEULEULEUALAPHELEUGLYVALALAVALTYRPHETYRCYSVALARGARGARGALAARGILEHISPHEMETLYSGLNPHEHISLYS

SEQ ID NO: 6 - protein, 453 aa, Homo sapiens
VALLEUHISGLYASNSERVALLEUGLNGLNTHRPROALATYRILELYSVALGLNTHRASNLYSMETVALMETLEUSERCYSGLUALALYSILESERLEUSERASNMETARGILETYRTRPLEUARGGLNARGGLNALAPROSERSERASPSERHISHISGLUPHELEUALALEUTRPASPSERALALYSGLYTHRILEHISGLYGLUGLUVALGLUGLNGLULYSILEALAVALPHEARGASPALASERARGPHEILELEUASNLEUTHRSERVALLYSPROGLUASPSERGLYILETYRPHECYSMETILEVALGLYSERPROGLULEUTHRPHEGLYLYSGLYTHRGLNLEUSERVALVALASPPHELEUPROTHRTHRALAGLNPROTHRLYSLYSSERTHRLEULYSLYSARGVALCYSARGLEUPROARGPROGLUTHRGLNLYSGLY

SEQ ID NO: 7 - DNA, 20 bp, Homo sapiens
  source 1..20: mol_type = other DNA
ccacattctgtggactggtg

SEQ ID NO: 8 - DNA, 22 bp, Homo sapiens
  source 1..22: mol_type = other DNA
attgatttcagctccacaactg

SEQ ID NO: 9 - DNA, 20 bp, Homo sapiens
  source 1..20: mol_type = other DNA
catgtgttcacctcagtggg

SEQ ID NO: 10 - DNA, 20 bp, Homo sapiens
  source 1..20: mol_type = other DNA
taccttaggccgtgtgatcc

SEQ ID NO: 11 - DNA, 25 bp, Homo sapiens
  source 1..25: mol_type = other DNA
cctgaccactgctgtttgttccaga

SEQ ID NO: 12 - DNA, 28 bp, Homo sapiens
  source 1..28: mol_type = other DNA
cagcaaagaaacatcaaggctcacagga

SEQ ID NO: 13 - DNA, 26 bp, Homo sapiens
  source 1..26: mol_type = other DNA
ttcaaacctcactcggagttcttggg

SEQ ID NO: 14 - DNA, 23 bp, Homo sapiens
  source 1..23: mol_type = other DNA
ttcttcacagcttgtctctgggc

SEQ ID NO: 15 - DNA, 24 bp, Homo sapiens
  source 1..24: mol_type = other DNA
agggctgccatgtatcctacagga

SEQ ID NO: 16 - DNA, 20 bp, Mus musculus
  source 1..20: mol_type = other DNA
ccagtttctccccacttgct

SEQ ID NO: 17 - DNA, 20 bp, Mus musculus
  source 1..20: mol_type = other DNA
aaccagctgtccaatgggag

SEQ ID NO: 18 - DNA, 20 bp, Mus musculus
  source 1..20: mol_type = other DNA
tgatgcagcctcacagtctg

SEQ ID NO: 19 - DNA, 20 bp, Mus musculus
  source 1..20: mol_type = other DNA
aaaggctgtggctgcttttg

SEQ ID NO: 20 - DNA, 26 bp, Mus musculus
  source 1..26: mol_type = other DNA
aaatttgtgcttcggaatgtggagga

SEQ ID NO: 21 - DNA, 22 bp, Mus musculus
  source 1..22: mol_type = other DNA
catcgctttatggatgtctctg

SEQ ID NO: 22 - DNA, 23 bp, Mus musculus
  source 1..23: mol_type = other DNA
tcagaagagcagccatgtcctgt

SEQ ID NO: 23 - DNA, 20 bp, Homo sapiens
  source 1..20: mol_type = other DNA
ccctgggcccactattgttt

SEQ ID NO: 24 - DNA, 20 bp, Homo sapiens
  source 1..20: mol_type = other DNA
agtggtgatgatgctggctt

SEQ ID NO: 25 - DNA, 20 bp, Homo sapiens
  source 1..20: mol_type = other DNA
ccctgggcccactattgttt

SEQ ID NO: 26 - DNA, 20 bp, Homo sapiens
  source 1..20: mol_type = other DNA
agtggtgatgatgctggctt

SEQ ID NO: 27 - DNA, 23 bp, Homo sapiens
  source 1..23: mol_type = other DNA
aaatccgcagctcgacgcagcat

SEQ ID NO: 28 - DNA, 20 bp, Homo sapiens
  source 1..20: mol_type = other DNA
tcaccagccgaggtactggg

SEQ ID NO: 29 - DNA, 26 bp, Mus musculus
  source 1..26: mol_type = other DNA
aaatttgtgcttcggaatgtggagga

SEQ ID NO: 30 - DNA, 22 bp, Mus musculus
  source 1..22: mol_type = other DNA
catcgctttatggatgtctctg

SEQ ID NO: 31 - DNA, 23 bp, Mus musculus
  source 1..23: mol_type = other DNA
tcagaagagcagccatgtcctgt

SEQ ID NO: 32 - DNA, 33 bp, synthetic construct
  source 1..33: mol_type = other DNA
aagcagtggtatcaacgcagagtacatrgrgrg

SEQ ID NO: 33 - DNA, 27 bp, synthetic construct
  source 1..27: mol_type = other DNA
aagcagtggtatcaacgcagagtacat

SEQ ID NO: 34 - DNA, 25 bp, synthetic construct
  source 1..25: mol_type = other DNA
ctgggagagatgacaatagcaggat

SEQ ID NO: 35 - DNA, 23 bp, synthetic construct
  source 1..23: mol_type = other DNA
cccagaatcgtgttgctcttctt

SEQ ID NO: 36 - DNA, 57 bp, synthetic construct
  source 1..57: mol_type = other DNA
gtgactggagttcagacgtgtgctcttccgatctaagcagtggtatcaacgcagagt

SEQ ID NO: 37 - DNA, 58 bp, synthetic construct
  source 1..58: mol_type = other DNA
acactctttccctacacgacgctcttccgatctaaacggatggtttggtatgaggctg

SEQ ID NO: 38 - DNA, 58 bp, synthetic construct
  source 1..58: mol_type = other DNA
acactctttccctacacgacgctcttccgatctgaaggaagaaaaatagtgggcttgg

SEQ ID NO: 39 - DNA, 21 bp, synthetic construct
  source 1..21: mol_type = other DNA
actacgattccatcaatgcct

SEQ ID NO: 40 - DNA, 21 bp, synthetic construct
  source 1..21: mol_type = other DNA
cttatcgatgataagctgtca

SEQ ID NO: 41 - DNA, 20 bp, synthetic construct
  source 1..20: mol_type = other DNA
ccacctcagcctggatttca

SEQ ID NO: 42 - DNA, 20 bp, synthetic construct
  source 1..20: mol_type = other DNA
tacattggtgctgggagtgg